(19) United States
(12) Patent Application Publication
(10) Pub. No.: US 2006/0148057 A1
(43) Pub. Date: Jul. 6, 2006

(54) Thioredoxin mutants and uses thereof

(76) Inventors: Wang Min, Pittsford, NY (US); Yingmei Liu, San Diego, CA (US)

Correspondence Address: NEEDLE & ROSENBERG, P.C., SUITE 1000, 999 PEACHTREE STREET, ATLANTA, GA, 30309-3915, US

(21) Appl. No.: 10/523,343

(22) PCT Filed: Jul. 22, 2003
(86) PCT No.: PCT/US03/22847

§ 371 Date: Aug. 26, 2005

(30) Foreign Application Priority Data
Aug. 2, 2002 (US) 60401073

(51) Int. Cl. C12N 9/06

(52) U.S. Cl. 435/191

(57) ABSTRACT

The invention relates to an isolated mutant thioredoxin (Trx) molecules and related molecules, including, for example, a thioredoxin mutant resistant to the oxidizing effects of cytokines or reactive oxygen species. The invention further provides various uses, including treatment and diagnostic uses.

### FIELD OF THE INVENTION

The present invention relates to mutant thioredoxin molecules and related molecules, antibodies directed to same, nucleic acids encoding same, and uses thereof.

### BACKGROUND OF THE INVENTION

Important cellular antioxidant systems include glutathione, superoxide dismutase (SOD) and thioredoxin (Trx). Thioredoxin (Trx) is a small multifunctional protein that acts as a cellular redox enzyme. Trx plays multiple functions in regulation of cell growth, apoptosis and activation (Powis et al. The role of the redox protein thioredoxin in cell growth and cancer. Free Radic Biol Med 29:312-22 (2000)) and may play a role in various diseases. Trx is also present in mitochondria.

Trx contains two redox-active cysteine residues in its catalytic center, having consensus amino acid sequence -cys32-gly-pro-cys35 (SEQ ID NO:15) (Holmgren, Thioredoxin. Annu Rev Biochem 54:237-71 (1985); Holmgren, Thioredoxin and glutaredoxin systems. J Biol Chem 264:13963-6 (1989)). Trx exists either in a reduced form with dithiol or in an oxidized form, in which C32 and C35 residues form an intramolecular disulfide bridge. A conserved region is present in mitochondrial Trx as well at residues C90 and C93. Trx participates in redox reactions by reversible oxidation of its active center dithiol to disulfide and catalyzes dithio-disulfide exchange reactions involving many thiol-dependent processes (Powis et al. The role of the redox protein thioredoxin in cell growth and cancer. Free Radic Biol Med 29:312-22 (2000); Holmgren, Thioredoxin. Annu Rev Biochem 54:237-71 (1985); Holmgren, Thioredoxin and glutaredoxin systems. J Biol Chem 264:13963-6 (1989)).

Various extracellular stimuli including growth factors, cytokines, and stress trigger MAP3K-MAP2K-MAPK cascades leading to cell growth, differentiation and apoptosis (Ip and Davis, Signal transduction by the c-Jun N-terminal kinase (JNK)—from inflammation to development. Curr Opin Cell Biol 10:205-19 (1998); Davis, Signal transduction by the JNK group of MAP kinases. Cell 103:239-52. (2000)). Apoptosis signal-regulating kinase 1 (ASK1) is one member of the MAPKKK (mitogen-activated protein kinase kinase kinase) family which are activated in response to proinflammatory and stress signals (Ichijo, From receptors to stress-activated MAP kinases. Oncogene 18:6087-93 (1999)). ASK1 was initially identified as an apoptosis-inducing kinase (Ichijo et al., Induction of apoptosis by ASK1, a mammalian MAPKKK that activates SAPK/JNK and p38 signaling pathways. Science 275:90-4 (1997); Chang et al., Activation of apoptosis signal-regulating kinase 1 (ASK1) by the adapter protein Daxx. Science 281:1860-3 (1998)). Recently it has been implicated in various cell functions including cell survival (Takeda et al., Apoptosis signal-regulating kinase 1 (ASK1) induces neuronal differentiation and survival of PC12 cells. J Biol Chem 275:9805-13 (2000)); differentiation (Sayama et al., Apoptosis signal-regulating kinase 1 (ASKI) is an intracellular inducer of keratinocyte differentiation. J Biol Chem 276:999-1004 (2001)); and inflammation (Liu et al., Laminar flow inhibits TNF-induced ASK1 activation by preventing dissociation of ASK1 from its inhibitor 14-3-3. J Clin Invest 107:917-23 (2001)). ASK1 is a 170 kD protein that functionally is composed of an inhibitory N-terminal domain, an internal kinase domain, and a C-terminal regulatory domain (Ichijo et al., Induction of apoptosis by ASK1, a mammalian MAPKKK that activates SAPK/JNK and p38 signaling pathways. Science 275:90-4 (1997); Chang et al., Activation of apoptosis signal-regulating kinase 1 (ASK1) by the adapter protein Daxx. Science 281:1860-3 (1998); Hatai et al., Execution of apoptosis signal-regulating kinase 1 (ASK1)-induced apoptosis by the mitochondria-dependent caspase activation. J Biol Chem 275:26576-81 (2000); Tobiume et al., ASK1 is required for sustained activations of JNK/p38 MAP kinases and apoptosis. EMBO Rep 2:222-8 (2001)). The C-terminal domain of ASK1 binds to the TRAF-domain and this association is required for ASK1 activation by cytokines (Nishitoh et al., ASK1 is essential for JNK/SAPK activation by TRAF2. Mol Cell 2:389-95 (1998)). The C-terminal domain also contains a phosphoserine site at Ser-967 through which 14-3-3 binds to ASK1 (Zhang et al., Suppression of apoptosis signal-regulating kinase 1-induced cell death by 14-3-3 proteins. Proc Natl Acad Sci U SA. 96:8511-5 (1999)). ASK1-induced apoptosis has been studied recently (Ichijo et al., Induction of apoptosis by ASK1, a mammalian MAPKKK that activates SAPK/JNK and p38 signaling pathways. Science 275:90-4 (1997); Chang et al., Activation of apoptosis signal-regulating kinase 1 (ASK1) by the adapter protein Daxx. Science 281:1860-3 (1998); Hatai et al., Execution of apoptosis signal-regulating kinase 1 (ASK1 )-induced apoptosis by the mitochondria-dependent caspase activation. J Biol Chem 275:26576-81 (2000); Tobiume et al., ASK1 is required for sustained activations of JNK/p38 MAP kinases and apoptosis. EMBO Rep 2:222-8 (2001); Zhang et al., Suppression of apoptosis signal-regulating kinase 1-induced cell death by 14-3-3 proteins. Proc Natl Acad Sci USA 96:8511-5 (1999)), and it has been reported that 14-3-3 inhibits ASK1-induced apoptosis (Zhang et al., Suppression of apoptosis signal-regulating kinase 1-induced cell death by 14-3-3 proteins. Proc Natl Acad Sci USA 96:8511-5 (1999)). The 14-3-3 has recently ben shown to be an important regulator in laminar flow-mediated inhibition of TNF-induced ASK1-JNK activation (Liu et al., Laminar flow inhibits TNF-induced ASK1 activation by preventing dissociation of ASK1 from its inhibitor 14-3-3. J Clin Invest 107:917-23 (2001)).

It has been shown that Trx associates with ASK1 at the N-termiinus and inhibits ASK1 activity (Saitoh et al., Mammalian thioredoxin is a direct inhibitor of apoptosis signal- regulating kinase (ASK) 1. Embo J 17:2596-606 (1998)). Deletion of the N-terminal 648 amino acids of ASK1 (ASK1-ΔN) leads to constitutive ASK1 kinase activity as it does in other MAP3Ks, confirming that Trx inhibits ASK1 via the N-terminal inhibitory domain (Saitoh et al., Mammalian thioredoxin is a direct inhibitor of apoptosis signal- regulating kinase (ASK) 1. Embo J 17:2596-606 (1998)). Trx in a reduced form binds to the N-terminal part of ASK1 and blocks activation of ASK1 by TNF (Saitoh et al., Mammalian thioredoxin is a direct inhibitor of apoptosis signal-regulating kinase (ASK) 1. Embo J 17:2596-606 (1998); Gotoh et al., “Reactive oxygen species-and dimerization-induced activation of apoptosis signal-regulating kinase 1 in tumor necrosis factor-alpha signal transduction.” J Biol Chem 273:17477-82 (1998)); Liu et al., “Activation of apoptosis signal-regulating kinase 1 (ASK1) by tumor necrosis factor receptor-associated factor 2 requires prior dissociation of the ASK1 inhibitor thioredoxin. “Mol Cell Biol 20:2198-208 (2000)). The oxidized form (intramolecular disulfide bond between C32 and C35) does not bind to ASK1. Apoptotic stimuli (TNF, ROS or serum starvation) activate ASK1 in part by oxidizing Trx to release Trx from ASK1 (Saitoh et al., Mammalian thioredoxin is a direct inhibitor of apoptosis signal-regulating kinase (ASK) 1. Embo J17:2596-606 (1998); Gotoh et al., “Reactive oxygen species- and dimerization-induced activation of apoptosis signal-regulating kinase 1 in tumor necrosis factor-alpha signal transduction.” J Biol Chem 273:17477-82 (1998); Liu et al., “Activation of apoptosis signal-regulating kinase 1 (ASK1 ) by tumor necrosis factor receptor-associated factor 2 requires prior dissociation of the ASK1 inhibitor thioredoxin.” Mol Cell Biol 20:2198-208 (2000)). The mechanism by which Trx inhibits ASK1 activity has not been determined. Thus, methods of analyzing and manipulating the interaction are needed in the art.

### BRIEF DESCRIPTION OF THE DRAWINGS

FIG. 1 shows Trx promotion of ASK1 ubiquitination and degradation. FIG. 1A shows that Trx promotes ASK1 ubiquitination and degradation. BAEC were transfected with Trx expression construct or a control vector (VC). Endogenous ASK1 and transfected Trx proteins were determined by Western blot with anti-ASK1 and anti-Flag, respectively. FIG. 1B shows assembly of poly-Ub into ASK1 molecule. ASK1 ubiquitination was determined by immunoprecipitation with anti-ASK1 (Santa Cruz) followed by Western blot with anti-Ub (Santa Cruz). The poly-Ub chains are indicated by a bracket. FIG. 1C shows that Trx does not induce ubiquitination and degradation of ASK1-ΔN. BEAC were co-transfected with ASK1-ΔN and Trx-expressing construct or a control vector. Expression of ASK1-ΔN and Trx was determined by Western blot with anti-Flag.

FIG. 2 shows TNF treatment and TRAF2 expression block Trx-induced ASK1 ubiquitination and degradation. BAEC were transfected and treated as follows: lane 1: VC; lane 2: Trx construct; lane 3: Trx and TRAF2 constructs; lane 4: Trx construct with TNF treatment. TNF (10 ng/ml for 15 min) was added at 24 h post-transfection. FIG. 2A shows expression of transfected Trx and TRAF2 by Western blot with anti-Flag. FIG. 2B shows endogenous ASK1 was determined by Western blot with anti-ASK1.

FIG. 3 shows that the redox activity of Trx is not required for induction of ASK1 ubiquitination and degradation. BAEC were transfected with Flag-tagged Trx-WT, Trx-CS, Trx-C32S, Trx-C35S-expressing constructs or VC as indicated. FIG. 2A shows expression of Trx as determined by Western blot with anti-Flag. FIG. 2B shows ASK1 protein determination with anti-ASK1. The poly-Ub chains above ASK1 protein are indicated by a bracket.

FIG. 4 shows that Trx-C32S and Trx-C35S, but not Trx-WT, associate with ASK1 in the presence of H₂O₂. FIG. 4A shows purification of GST-Trx fusion proteins. GST-Trx-WT, CS, C32S and C35S expressed in E. coli and purified by glutathione agarose beads. GST-Trx proteins were examined by SDS-PAGE followed by Commassie blue staining. FIG. 4B shows in vitro GST-Trx binding assay. HA-tagged ASK1-WT was transfected into BAEC. Cell lysates were applied to GST-Trx protein beads (Trx-WT, CS, C32S, and C35S) in the presence of DTT (lanes 1-5) or 1 mM H₂O₂ (lanes 6-10). The bound ASK1 was determined by Western blot with anti-HA. GST was used as a control.

FIG. 5 shows association of Trx-C32S and Trx-C35S with ASK1 in vivo is resistant to TNF treatment. TNF activates ASK1 in part by dissociating Trx from ASK1 in a ROS-dependent manner. BAEC were subjected to the following treatment: untreated (Ctrl), TNF-α (10 ng/ml) stimulation for 15 min (TNF), N-acetyl-cysteine (1 mM) for 60 min (Nac), Nac for 45 min followed by TNF for 15 min (Nac+TNF). FIG. 5A shows that ASK1 activation by TNF is ROS-dependent. Cell lysates were prepared and analyzed for ASK activity by an in vitro kinase assay using GST-MKK4 as a substrate. FIG. 5B shows TNF induces dissociation of Trx from ASK1. Cell lysates were immunoprecipitated with anti-Trx followed by Western blot with anti-ASK1 (top panel). Trx protein in the immunoprecipitates was determined with anti-Trx (lower panels). FIG. 5C shows that association of Trx-C32S and Trx-C35S with ASK1 in vivo is resistant to TNF treatment. HA-tagged ASK1-WT was co-transfected with Flag-tagged Trx constructs (WT, CS, C32S or C35S). 24 h post-transfection, cells were either lef untreated (Control) or treated with TNF (10 ng/ml) for 15 min prior to harvest. Cell lysates were immunoprecipitated by anti-Flag. ASK1-WT was detected by Western blot with anti-HA and Trx proteins by anti-Flag.

FIG. 6 shows the effects of Trx on ASK1 activity. Trx inhibits ASK1-induced JNK activation and caspase 3 activity. BAEC were co-transfected with ASK1 and Trx constructs in 1:1 ratio as indicated: control vector alone (Ctrl), ASK1 with VC, Trx-WT, Trx-CS, Trx-C32S or Trx-C35S. 24 h post-transfection, cell lysates were used to examine JNK and caspase 3 activity. FIG. 6A shows that Trx inhibits ASK1-induced JNK activation. JNK activity was measured by an in vitro kinase assay GST-c-Jun as a substrate. Trx expression was determined by Western blot anti-Flag. FIG. 6B shows that Trx inhibits ASKl -induced caspase 3 activation. Caspase 3 activity was measured by Sigma's Caspase 3 Fluorometric Assay Kit in the absence or presence of caspase 3 inhibitor Ac-DEVD-CHO. Assay was performed as duplicates. Caspase 3 activities were normalized with the Ctrl group (as zero). Data are presented from mean of two independent experiments. FIG. 6C shows Trx-C32S and Trx-C35S inhibition of ASK1-induced apoptosis in a TNF-resistant manner. 6-well BAEC were co-transfected with a GFP expression construct and ASK1/Trx constructs as indicated (1 μg GFP, 1 μg ASK1, 1 μg Trx, total DNA was kept to 3 μg by adding VC): control vector alone (Ctrl), ASK1 with VC, Trx-WT, Trx-CS, Trx-C32S or Trx-C35S. Cells were cultured in the presence or absence of TNF (10 ng/ml). 48 h after transfection, GFP-positive cells were counted as survival cells. The survival rate is shown (Ctrl group as 100%). Data are presented from mean of four independent experiments. FIG. 6D shows that Trx-C32S and Trx-C35S, but not Trx-WT, inhibits TNF+CHX-induced apoptosis. BAEC were transfetced as in FIG. 6C. 6-well BAEC were transfetced with GFP (1 μg) and Trx -expressing construct (1 μg each) as indicated. 24 h after transfection, cells were either left untreated or treated with TNF (10 ng/ml) plus CHX (10 μg/ml) for 24 h. GFP-positive cells were counted as survival cells. The survival rate is shown (All untreated cells had a similar survival rate and we take untreated VC as 100%, Ctrl). Data are presented from mean of four independent experiments.

FIG. 7 shows a model for regulation of ASK1 by Trx. FIG. 7A shows that the wild-type Trx binds to ASK1 in a reduced form and inhibits ASK1 activity. Apoptotic stimuli activate ASK1 by oxidizing and dissociating Trx from ASK1 leading to apoptosis. FIG. 7B shows that the single-mutant Trx at the catalytic sites (C32S or C35S) retains the binding ability for ASK1. Trx-C32S and Trx-C35S are resistance to oxidization by TNF/ROS leading to a constitutive inhibition on ASK1-mediated apoptosis induced by TNF/ROS.

FIG. 8 shows ASK1 inhibited IGF-1-induced phosphorylation of IRS-1, Akt and eNOS activity. EC were transfected as indicated: VC, ASK1 or SOCS3. 24 h post-transfection, cells were either treated with IGF-1 (10 ng/ml) for 10 min (A and B) and 12 h (C). FIG. 8A shows IRS-phosphorylation determined by imunoprecipitated with anti-IRS-1 followed by Western blot with anti-phosphotyrosine (4G10). Total IRS-1 protein in the IP was determined by Western blot with anti-IRS-1. FIG. 8B shows Western blots of cell lysates with phospho-Akt (Ser473)-specific antibody (p-Akt). Total Akt was determined by Western blot with anti-Akt. FIG. 8C shows the results of an eNOS activity assay performed according to the protocol provided by the Manufacturer (Calbiochem, Nitric Oxide Synthase Assay Kit). Fold increases in eNOS activity is shown. Data presented are average from two independent experiments.

FIG. 9 shows Trx prevents ASK1-induced inhibition of Akt phosphorylation. EC were transfected with plasmids as indicated: control vector (VC), HA-Akt, HA-ASK1 with different Trx constructs. Cells were treated with IGF1 (10 ng/ml for 10 min). Akt hosphorylation was examined by western blot with phospho-Akt (Ser473)-specific antibody (p-Akt). Akt and ASK1 expression were determined by western blot with anti-HA.

FIG. 10A shows statins and TZDs inhibit TNF-induced ASK1 activation. HUVEC were pretreated with DMSO (mock), simvastatin (1 μM, Statin) and rosiglitazone (5 μM, TZD) for 30 min followed by TNF for 15 min. Cell lysates were examined for ASK1 activity by an in vitro kinase assay using GST-JNKK1 as a substrate. FIG. 10B shows a general model for anti-atherosclerotis agents inhibition on ASK1. Laminar flow, TZDs and statins inhibit TNF-induced ASK1 activation to restore insulin signaling.

FIG. 11 shows ASK1 phosphorylates SOCS3 at the SOCS box and stablizes SOCS3 in a kinase activity-dependent manner. FIGS. 11A and B show ASK1 stabilizes endogenous SOCS3 in EC. FIG. 11B shows ASK1-induced SOCS3 stabilization is kinase activity-dependent. FIG. l lC shows both SH2 domain and the SOCS box are responsible for ASK1 association. FIG. 11D shows ASK1 phosphorylates SOCS3 at the SOCS box.

FIG. 12 shows specific expression of Trx-C35S in EC driven by Tie2 promoter. FIG. 12A shows a schema of transgenic vector showing inserted Flag-tagged human Trx-C35S and location of primers used for genotyping by PCR. FIG. 12B shows BAEC were transfected with Tie2 vector (VC) or Tie2-Trx-C35S. Detection of Trx-C35S in BAEC by anti-Trx (Medical & Biological Laboratory) which recognizes both endogenous and transfected Trx. FIG. 12C shows detection of Tix-C35S by Western blot with anti-Flag.

FIG. 13 shows ASK1 specifically interacts with cTnT in yeast two-hybrid system. FIG. 13A shows expression of ASK1 -AN in yeast. Yeast cells harboring ASK1-ΔN expression plasmid or pAS2.1 (VC) were used for Western blot with anti-Gal4 antibody. FIG. 13B shows the interaction of ASK1-AN with cTnT. Yeast transformants were grown on QDO (Ade⁻, Leu⁻, Trp⁻, His⁻) plates followed by the β-galactosidase filter assay. Interaction of two proteins (ASK1-ΔN and cTnT) permitted yeast to grow on QDO plate and became positive in β-galactosidase activity. Control vector (VC) with cTnT did not grow.

FIG. 14 shows cTnT directly interacts with the C-terminal domain ofASK1 in vitro. FIG. 14A shows a schematic diagram ofASK1 domains. ASK1-N comprises amino acids 1-678, ASK1-K comprises amino acids 678-936, and ASK1-ΔN comprises amino acids 678-1375. Figures B and C show association of cTnT with the C-terminal domain of ASK1. HA-ASK1-WT, Flag-ASK1-ΔN, ASK1-N or ASK1-K was transiently transfected in 293T cells. Association of ASK1 proteins with cTnT was determined by an in vitro binding to GST-cTnT followed by Western blot with either anti-HA (ASK1 -WT) or anti-Flag (for ASK1 -ΔN, ASK1-N and ASK1-K). GST was used as a control.

FIG. 15 shows ASK1 interacts with cTnT in vivo. FIG. 3A shows expression of cTnT and ASK1. 293T cells were transiently transfected with Flag-cTnT and HA-ASK1 expression plasmids as indicated. Expression of cTnT and ASK1 was examined by Western blot with anti-Flag and anti-HA, respectively. FIG. 15B and C shows association of cTnT with ASK1 in vivo. Association of cTnT with ASK1 was determined by immunoprecipitation (IP) with anti-Flag antibody followed by immunoblot (IB) with anti-HA (FIG. 15B). Association of cTnT with ASK1 was also determined by IP with anti-HA antibody followed by 113 with anti-Flag (FIG. 15C).

FIG. 16 shows ASK1 associates with cTnT in cardiomyocytes. Neonatal rat cardiomyocytes were isolated and cultured as described. Association of endogenous ASK1 and cTnT was determined by IP with anti-cTnT (Santa Cruz) followed by IB with anti-ASK1 (H300, Santa Cruz) (FIG. 16A). Association of endogenous ASK1 and cTnT was determined by IP with anti-ASK1 followed by IB with anti-cTnT. Normal rabbit serum (NS) was used as a control.

FIG. 17 shows ASK1 phosphorylates cTnT in cardiac troponin protein complex (cTnT/I/C) in vitro. FIG. 17A shows detection of the purified cardiac troponin proteins Research Diagnosis). 10 μg of cTn protein complex was run on 10% SDS-PAGE followed by Conunassie staining. cTnT (39 kDa), cTnC (29 kDa) and cTnI (18 kDa) are indicated. FIG. 17B shows cTnT, but not cTnC or cTnI, is phosphorylated by ASK1-ΔN. ASK1-ΔN protein was IP from ASK1-ΔN-expressing 293T cells with anti-Flag and normal serum (NS) was used as a control. Phosphorylation of cTn proteins by the ASK1-ΔN immunoprecipitate was examined by an in vitro kinase assay using the cTn complex as a substrate (+, lanes 2 and 4). No cTn complex was used as controls (−, lanes 1 and 3). Autoradiogram shown is representative of two similar experiments. cTnT phosphorylation by ASK1-ΔN and ASK1-ΔN autophosphorylation are indicated.

FIG. 18 shows ASK1 phosphorylates cTnT in vivo. FIG. 18A shows overexpression of cTnT and ASK1-ΔN. 293T cells were transiently transfected with Flag-cTnT and ASK1-ΔN expression plasmids as indicated. Expression of cTnT and ASK1-ΔN was determined by Western blot with anti-Flag. FIG. 18 B shows phosphorylation of cTnT by ASK1-ΔN in vivo. Cells were labeled with ³²P-orthophosphate as described below. Flag-cTnT and ASK1-ΔN proteins were immunoprecipitated by anti-Flag and phosphorylation of cTnT was examined by SDS/PAGE. Autoradiogram shown is representative of two similar experiments. cTnT phosphorylation by ASK1-ΔN and ASK1-ΔN autophosphorylation are indicated.

FIG. 19 shows ASK1 phosphorylates cTnT at a consensus motif ofASK1 phosphorylation sites (T194/S198). Mutation of a consensus motif (T/SxxxT/S) of ASK1 phosphorylation sites in cTnT was generated (cTnT-TS/AA) and phosphorylation of cTnT-TS/AA was determined in vitro and in vivo. FIG. 19A shows mutation at T194/S198 sites significantly decreased cTnT phosphorylation by ASK1-ΔN in vitro. Phosphorylation of cTnT by immunoprecipitated Flag- ASK1-ΔN was examined in an in vitro kinase assay using purified GST-cTnT or cTnT-TS/AA as a substrate. 10 μg of GST-cTnT was mixed with the ASK1-ΔN immunoprecipitate in a kinase buffer containing 0.5 μCi [γ-³²P]ATP. The sample was incubated at 30° C. for 30 min followed by SDS-PAGE and autoradiography. GST proteins was visualized by Western blot with anti-GST. Anormal serum (NS) was used as a control. FIG. 19B shows mutation at T194/S198 sites significantly decreased cTnT phosphorylation by ASK1-ΔN in vivo. Flag-tagged expression plasmids for ASK1-ΔN and the wild-type cTnT or mutant cTnT (cTnT-TS/AA) were co-transfected in 293T. Phosphorylation of cTnT by ASK1-ΔN was determined by IP with anti-Flag followed by an in vitro kinase assay. Empty vector (VC) was used as a control for ASK1-ΔN expression vector. Autoradiogram shown is representative of two similar experiments. Ratio of phosphorylated cTnT to expressed cTnT (p-cTnT/cTnT) was quantitated by densitometry. Protein expression of cTnT and ASK1 was determined by Western blot with anti-Flag. Data presented in the text are the average of two experiments.

FIG. 20 shows that ROS induces activation of ASK1, cTnT phosphorylation and inhibition of contractility in cardiomyocytes. Neonatal and adult rat cardiomyocytes were cultured in 6-well plates and were either untreated (Ctrl) or treated H₂O₂ (100 μM for 1, 5 or 15 min). ASK1 activity, cTnT phosphorylation and cardiac contractility were assayed. FIG. 20A shows H₂O₂ induces activation of ASK1. ASK1 activity was measured by IP with anti-ASK1 followed by an in vitro kinase assay using GST-MKK4 as a substrate. Autoradiogram shown is representative of three similar experiments. FIG. 20B shows H₂O₂ induces cTnT phosphorylation. Cells were labeled with ³²P-orthophosphate as described. cTnT protein was IP by anti-cTnT and phosphorylation of cTnT was examined by SDS/PAGE. Autoradiogram shown is representative of two similar experiments.

FIG. 21 shows overexpression of constitutively active ASK1 in cardiomyocytes inhibits MgATPase activity and decreases cardiomyocyte contractility. Neonatal rat cardiomyocytes were infected with either empty vector (VC) or ASK1-ΔN-expressing lentivirus at MOI of 50. 24 h post-infection, ASK1-ΔN expression, activity, MgATPase and contractile activity were determined as described. FIG. 21A shows ASK1-ΔN expression in cardiomycytes. Infection efficiency of cardiomyocytes by the HIV-derived lentiviral system was determined for GFP-positive cells under a fluorescence microscope. Expression of ASK1-ΔN by Western blot with anti-Flag. FIG. 21B shows ASK1-ΔN is active in cardiomyocytes. ASK1 activity was measured for ASK1 activity using the in vitro kinase assay using GST-MKK4 as a substrate. FIG. 21C shows inhibition of MgATPase activity by ASK1-ΔN expression. MgATPase activity was determined by measuring release of free Pi using ³²P-γ-ATP as a substrate as described in Method. Total MgATPase activity was determined in the assay buffer containing 4.86 μM CaCl₂. Basal MgATPase level was determined in the same MgTAPase assay buffer except that 4.86 μM CaCl₂ was replace by 1.6 mM EGTA. Each sample was performed as duplicates. Data presented are averages of the two experiments. * Significant difference from vector control, p<0.05. FIG. 21D shows inhibition of contractility of cardiomyocyte by ASK1. Contractility of cardiomyocytes was measured by counting number of contractile cells under fluorescence microscope. Data presented are numbers of GFP-positive and contractile cells per field (4×) from two independent experiments. * Significant difference from vector control,p<0.05.

### DETAILED DESCRIPTION OF THE INVENTION

In this specification and in the claims that follow, reference will be made to a number of terms that shall be defined to have the following meanings:

As used in the specification and the appended claims, the singular forms “a,” “an” and “the” include plural referents unless the context clearly dictates otherwise. Thus, for example, reference to a receptor includes mixtures of various receptors, reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

As used throughout, by “subject” is meant an individual. Preferably, the subject is a mammal such as a primate, and, more preferably, a human. The term “subject” can include domesticated animals, such as cats, dogs, etc., livestock (e.g., cattle, horses, pigs, sheep, goats, etc.), and laboratory animals (e.g., mouse, rabbit, rat, guinea pig, etc.).

Polypeptides

The invention relates to an isolated mutant thioredoxin (Trx) molecule, wherein the thioredoxin molecule is resistant to the oxidizing effects of cytokines or reactive oxygen species. The mutant thioredoxin molecule can be a mutant of the wild type cellular Trx or mitochondrial Trx. As referred to herein, a particular residue of Trx is based on the residues numbers of the cellular Trx. The mutation in one embodiment comprises a mutation at the catalytic site. The catalytic site in both cellular and mitochondrial Trx comprises CGPC (SEQ ID NO:15). The catalytic site corresponds to residues C32 through C35 in cellular Trx and is conserved in mitochondrial Trx. The amino acid alteration in a preferred embodiment of the mutant Trx decreases the number of effective sulfhydryl groups in the molecule as compared to the wild type thioredoxin. Thus, in a preferred embodiment, the mutation is a point mutation in the amino acid sequence. More specifically, the amino acid alteration is preferably a cysteine substitution (e.g., a cysteine to serine substitution) or a cysteine deletion, including, for example, a substitution or deletion at either residue 32 or residue 35 of Trx. Thus the catalytic site of the mutant Trx can comprise XGPC (SEQ ID NO:16) or CGPX (SEQ ID NO:17), wherein X is any residue except cysteine. Even more specifically, the mutant Trx can have the amino acid sequence of SEQ ID NO:2 or 3. The mutant Trx in another embodiment has the amino acid sequence of SEQ ID NO:10 or SEQ ID NO:11. In yet another embodiment the mutant further comprises a substitution or deletion mutation in the cysteine residue at position 69. Thus, the mutant can have the amino acid sequence of SEQ ID NO:18 or SEQ ID NO:19, or other comparable sequences in which a serine residue replaces the C69 residue. Other amino acid substitutions other than serine could similarly be used.

A mutant Trx with a single-mutation at the catalytic site (e.g., Trx-C32S or Trx-C35S) binds to ASK1 or proteins of other redox regulated pathways, like p53 and NF-κB. As used throughout, when ASK1 is referenced, it is used as an example and it is understood that either p53 or NF-κcB could replace the ASK1.

The redox activity of Trx is not required for the association of Trx with ASK1. One of the Cys residues (either C32 or C35) in Trx, however, is essential for the interaction of Trx with ASK1, and one cysteine residue is preserved in one embodiment of the mutant. The single Cys-containing Trx mutant forms a stable complex by intermolecular disulfide bridge with its enzyme Trx reductase (via C32) or its substrate transcription factor NF-κB (via C35). Trx forms this type of complex with ASK1 via either of the Cys residues. The association of Trx via one of the Cys with ASK1 is necessary and sufficient to promote ASK1 ubiquitination and degradation leading to decreased ASK1 apoptotic activity.

The crystal structure of Trx show that only C32 (in the C35S mutant) is exposed to solvent, suggesting C32 (in Trx-C35S) may be more accessible to ASK1 interaction. This explains why Trx-C35S has slightly stronger activity than Trx-C32S in ASK1 binding, induction of ASK1 ubiquitination/degradation and inhibition of ASK1-induced apoptosis.

Trx occurs naturally in many forms—mitochondrial and cellular, reduced and oxidized, full-length and truncated, intracellular and secreted forms. These different forms of Trx have different functions in a variety of physiological and pathological settings such as atherosclerosis and arthritis. Trx, for example, has a role in inhibiting apoptosis but certain forms are overexpressed in tumors, although the form of Trx differs in tumors. One example of Trx is the polypeptide having the amino acid sequence of SEQ ID NO:1. The various forms of Trx can include amino acid mutations and are referred to herein as “mutant Trxs.” A C32S mutant of the Trx having the amino acid of SEQ ID NO:1 has the amino acid sequence of SEQ ID NO:2. A C35S mutant of the Trx having the amino acid of SEQ ID NO:1 has the amino acid sequence of SEQ ID NO:3. Other forms of TRX can have similar mutations at one of the cysteine residues at the catalytic site. For example, the mt Trx can have the amino acid sequence of SEQ ID NO:9, and the C32S and C35S mutants thereof can have the amino acid sequences of SEQ ID NO:10 and SEQ ID NO: 11, respectively.

Truncated forms of Trx (i.e., fragments) include the catalytic region or any portion of at least 10 amino acids or any combination of the regions or portions. Trx variants are produced by making amino acid substitutions, deletions, and insertions, as well as post-translational modifications. Variations in post-translational modifications can include variations in the type or amount of carbohydrate moieties of the protein core or any fragment or derivative thereof. Variations in amino acid sequence may arise naturally as allelic variations (e.g., due to genetic polymorphism) or may be produced by human intervention (e.g., by mutagenesis of cloned DNA sequences), such as induced point, deletion, insertion and substitution mutants. These modifications can result in changes in the amino acid sequence, provide silent mutations, modify a restriction site, or provide other specific mutations.

Amino acid sequence modifications fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. Typically, no more than about 2 to 6 residues are deleted at any one site within the protein molecule. These variants ordinarily are prepared by site-specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known and include, for example, M13 primer mutagenesis and PCR mutagenesis. Amino acid substitutions are typically of single residues but may include multiple substitutions at different positions; insertions usually will be on the order of about from 1 to 10 amino acid residues but can be more; and deletions will range about from 1 to 30 residues, but can be more. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with Table 1 and are referred to as conservative substitutions.

TABLE 1
Amino Acid Substitutions

| | Exemplary |
|---|---|
| Original Residue | Substitutions |
| Ala | Ser |
| Arg | Lys |
| Asn | Gln |
| Asp | Glu |
| Cys | Ser |
| Gln | Asn |
| Glu | Asp |
| Gly | Pro |
| His | Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 1, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions that in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

In all mutational events, it is understood that the controlling aspect of the mutation is the function that the subsequent protein possesses. Certain mutations are those that do not detectably change the desired function. Such mutations can be combined with the mutations that makes Trx resistant to the oxidizing effects of cytokines or reactive oxygen species. Other mutations can enhance the effects of the mutations that make Trx resistant to the oxidizing effects of cytokines or reactive oxygen species.

Also provided by the present invention are variants of the C32S and C35S mutants of Trx. For example, variants of the C32S and C35S mutants, which have the amino acid sequences of SEQ ID NO:2 and SEQ ID NO:3, respectively, include amino acid sequences with one or more conservative amino acid substitutions. The variants can include one, two, three, four, five, or more conservative amino acid substitutions.

The invention also provides polypeptides that are about 70, 75, 80, 85, 90, 95, 98, or 99% homologous to the sequences of SEQ ID NO:2 or SEQ ID NO:3. Homology is characterized by identity in amino acid residues, by proximity in the location of the genes and by similarities as identified in a composite analysis. As used herein, “percent homology” of two amino acid sequences or of two nucleic acid sequences is determined using the algorithm of Karlin and Altschul (Proc. Natl. Acad. Sci. USA 87:2264-2268 (1990)). Such an algorithm is incorporated into the NBLAST and XBLAST programs of Altschul et al. (J. Mol. Biol. 215:403-410 (1990)). BLAST nucleotide searches are performed with the NBLAST program, score 100, wordlength=12, to obtain nucleotide sequences homologous to a nucleic acid molecule of the invention. BLAST protein searches are performed with the XBLAST program, score=50, wordlength=3, to obtain amino acid sequences homologous to a reference polypeptide . To obtain gapped alignments for comparison purposes, Gapped Blast is utilized as described in Altschul et al. (Nucl. Acids Res. 25: 3389-3402 (1997)). When utilizing BLAST and Gapped BLAST programs, the default parameters of the respective programs (e.g., XBLAST and NBLAST) are used. See http://ww.ncbi.nlm.nih.gov.

As used throughout, “resistant” or “resistance” refers to a substantial decrease in a biological response to a stimulus. A substantial decrease includes a 75, 80, 85, 90, or 100% lessening in the response to a stimulus as compared to the control response. For example, a mutant Trx “resistant to the oxidizing effects of cytokines or reactive oxygen species” refers to a substantial decrease in the biological response to a cytokine or reactive species as compared to effect of of the cytokine or reactive species in the non-mutant (i.e., wildtype) Trx.

As used throughout, “cytokine” or “cytokines” with oxidizing effects on wildtype Trx include pro-inflammatory cytokines, including but not limited to TNF, interleukin (e.g., IL-1), and lipopolysaccharides (LPS).

As used throughout, “reactive oxygen species” refers to oxygen radicals. Such oxygen radicals include H₂O₂—, O₂—, OH—, and ONOO—.

By “oxidizing effects” is meant the effect of formation of di-sulfide bonds, for example, between the cysteine residues of the catalytic site of Trx and downstream effects there of. For example, when a disulfide bond is formed between the cysteine residues of the catalytic site of wildtype Trx, there is a decrease in the amount of Trx-ASK 1 binding. Thus, an oxidizing effect can be a decrease in the bound Trx.

The invention further provides a therapeutic composition of the mutant Trx of the invention or therapeutic compositions of a small molecule that blocks the thiol group of wild-type Trx. Such a composition typically contains from about 0.1 to 90% by weight (such as 1 to 20% or 1 to 10%) of a therapeutic agent of the invention in a pharmaceutically acceptable carrier.

Antibodies

The invention also provides a purified antibody or immunologic fragment thereof, wherein the antibody or fragment thereof selectively binds to the mutant Trx of the invention. In one embodiment, the antibody of the invention binds one mutant (e.g., C32S) but not another (e.g., C35S) As used herein, the term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(I)) followed by a number of constant domains. Each light chain has a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (k) and lambda (1), based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. The heavy chain constant domains that correspond to the different classes of immunoglobulins are called alpha, delta, epsilon, gamma, and mu, respectively.

The term “variable” is used herein to describe certain portions of the variable domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a b-sheet configuration, connected by three CDRs, which form loops connecting, and in some cases forming part of, the b-sheet structure. The CDRs in each chain are held together in close proximity by the FR regions and, with the CDRs from the other chain, contribute to the formation of the antigen binding site of antibodies (see Kabat E. A. et al., “Sequences of Proteins of Inmunological Interest” National Institutes of Health, Bethesda, Md. (1987)). The constant domains are not involved directly in binding an antibody to an antigen, but exhibit various effector functions, such as participation of the antibody in antibody-dependent cellular toxicity.

The term “antibody or fragments thereof” can also encompass chimeric antibodies and hybrid antibodies, with dual or multiple antigen or epitope specificities, and fragments, such as F(ab′)₂, Fab′, Fab and the like, including hybrid fragments. Thus, fragments of the antibodies that retain the ability to bind their specific antigens are provided. For example, fragments of antibodies which maintain Trx binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies) as described, for example, in U.S. Pat. No. 4,704,692, the contents of which are hereby incorporated by reference.

In one embodiment, the antibody is a monoclonal antibody. The term “monoclonal antibody” as used herein refers to an antibody obtained from a substantially homogeneous population of antibodies, i.e., the individual antibodies comprising the population are identical except for possible naturally occurring mutations that may be present in minor amounts. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired activity (See, U.S. Pat. No. 4,816,567 and Morrison et al., Proc. Natl. Acad. Sci. USA, 81:6851-6855 (1984)).

Monoclonal antibodies of the invention may be prepared using hybridoma methods, such as those described by Kohler and Milstein, Nature, 256:495 (1975) or Harlow and Lane, Antibodies, A Laboratory Manual. Cold Spring Harbor Publications, New York, (1988). In a hybridoma method, a mouse or other appropriate host animal, is typically immunized with an immunizing agent to elicit lymphocytes that produce or are capable of producing antibodies that will specifically bind to the immunizing agent. Alternatively, the lymphocytes may be immunized in vitro. Preferably, the immunizing agent comprises a mutant Trx. Traditionally, the generation of monoclonal antibodies has depended on the availability of purified protein or peptides for use as the immunogen. More recently DNA based immunizations have shown promise as a way to elicit strong immune responses and generate monoclonal antibodies. In this approach, DNA-based immunization can be used, wherein DNA encoding a portion of the mutant Trx, preferably the catalytic region, is injected into the host animal according to methods known in the art.

Generally, either peripheral blood lymphocytes (“PBLs”) are used in methods of producing monoclonal antibodies if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, “Monoclonal Antibodies: Principles and Practice” Academic Press, (1986) pp. 59-103). Immortalized cell lines are usually transformed mammalian cells, including myeloma cells of rodent, bovine, equine, and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies (Kozbor, J Immunol 133:3001 (1984); Brodeur et al., “Monoclonal Antibody Production Techniques and Applications” Marcel Dekker, Inc., New York, (1987) pp. 51-63).

The culture medium in which the hybridoma cells are cultured can then be assayed for the presence of monoclonal antibodies directed against the mutant Trx, and more prefereablydirected against either the C32S or C35S and not against the wildtype Trx. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art, and are described further in Harlow and Lane “Antibodies, A Laboratory Manual” Cold Spring Harbor Publications, New York, (1988).

After the desired hybridoma cells are identified, the clones may be subcloned by limiting dilution or FACS sorting procedures and grown by standard methods. Suitable culture media for this purpose include, for example, Dulbecco's Modified Eagle's Medium and RPMI-1640 medium. Alternatively, the hybridoma cells maybe grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, plasmacytoma cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567) or by covalentlyjoining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody comprising one antigen-combining site having specificity for Trx and another antigen-combining site having specificity for a different antigen.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994, U.S. Pat. No. 4,342,566, and Harlow and Lane, Antibodies, A Laboratory Manual, Cold Spring Harbor Publications, New York, (1988). Papain digestion of antibodies typically produces two identical antigen binding fragments, called Fab fragments, each with a single antigen binding site, and a residual Fc fragment. Pepsin treatment yields a fragment, called the F(ab′)₂ fragment, that has two antigen combining sites and is still capable of cross-linking antigen.

The Fab fragments produced in the antibody digestion also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)₂ fragment is a bivalent fragment comprising two Fab′ fragments linked by a disulfide bridge at the hinge region. Fab′-SH is the designation herein for Fab′ in which the cysteine residue(s) of the constant domains bear a free thiol group. Antibody fragments originally were produced as pairs of Fab′ fragments which have hinge cysteines between them. Other chemical couplings of antibody fragments are also known.

An isolated immunogenically specific epitope or fragment of the antibody is also provided. A specific immunogenic epitope of the antibody can be isolated from the whole antibody by chemical or mechanical disruption of the molecule. The purified fragments thus obtained can be tested to determine their immunogenicity and specificity by the methods taught herein. Immunoreactive epitopes of the antibody can also be synthesized directly. An immunoreactive fragment is defined as an amino acid sequence of at least about two to five consecutive amino acids derived from the antibody amino acid sequence.

One method of producing proteins comprising the antibodies of the present invention is to link two or more peptides or polypeptides together by protein chemistry techniques. For example, peptides or polypeptides can be chemically synthesized using currently available laboratory equipment using either Fmoc (9-fluorenylmethyl-oxycarbonyl) or Boc (tert-butyloxycarbonoyl) chemistry. (Applied Biosystems, Inc., Foster City, Calif.). One skilled in the art can readily appreciate that a peptide or polypeptide corresponding to the antibody of the present invention, for example, can be synthesized by standard chemical reactions. For example, a peptide or polypeptide can be synthesized and not cleaved from its synthesis resin whereas the other fragment of an antibody can be synthesized and subsequently cleaved from the resin, thereby exposing a terminal group that is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W. H. Freeman and Co., N.Y. (1992); Bodansky M and Trost B., Ed. (1993) Principles of Peptide Synthesis. Springer-Verlag Inc., NY).

Alternatively, the peptide or polypeptide can by independently synthesized in vivo as described above. Once isolated, these independent peptides or polypeptides may be linked to form an antibody or fragment thereof via similar peptide condensation reactions. For example, enzymatic ligation of cloned or synthetic peptide segments can allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide-α-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments can be chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

The invention also provides fragments of antibodies that have bioactivity. The polypeptide fragments of the present invention can be recombinant proteins obtained by cloning nucleic acids encoding the polypeptide in an expression system capable of producing the polypeptide fragments thereof, such as an adenovirus or baculovirus expression system. For example, one can determine the active domain of an antibody from a specific hybridoma that can cause a biological effect associated with the interaction of the antibody with the mutant Trx. For example, amino acids found to not contribute to either the activity or the binding specificity or affinity of the antibody can be deleted without a loss in the respective activity.

For example, amino or carboxy-terminal amino acids can be sequentially removed from either the native or the modified non-immunoglobulin molecule or the immunoglobulin molecule and the respective activity assayed in one of many available assays. In another example, a fragment of an antibody can comprise a modified antibody wherein at least one amino acid has been substituted for the naturally occurring amino acid at a specific position, and a portion of either amino terminal or carboxy terminal amino acids, or even an internal region of the antibody, has been replaced with a polypeptide fragment or other moiety, such as biotin, which can facilitate in the purification of the modified antibody. For example, a modified antibody can be fused to a maltose binding protein, through either peptide chemistry of cloning the respective nucleic acids encoding the two polypeptide fragments into an expression vector such that the expression of the coding region results in a hybrid polypeptide. The hybrid polypeptide can be affinity purified by passing it over an amylose affinity column, and the modified antibody receptor can then be separated from the maltose binding region by cleaving the hybrid polypeptide with the specific protease factor Xa. (See, for example, New England Biolabs Product Catalog, 1996, pg. 164.). Similar purification procedures are available for isolating hybrid proteins from eukaryotic cells as well.

The fragments, whether attached to other sequences, can also include insertions, deletions, substitutions, or other selected modifications of particular regions or specific amino acids residues, provided the activity of the fragment is not significantly altered or impaired compared to the nonmodified antibody or antibody fragment. These modifications can provide for some additional property, such as to remove or add amino acids capable of disulfide bonding, to increase its bio-longevity, to alter its secretory characteristics, etc. In any case, the fragment must possess a bioactive property, such as binding activity, regulation of binding at the binding domain, etc. Functional or active regions of the antibody may be identified by mutagenesis of a specific region of the protein, followed by expression and testing of the expressed polypeptide. Such methods are readily apparent to a skilled practitioner in the art and can include site-specific mutagenesis of the nucleic acid encoding the antigen. (Zoller M J et al. Nucl. Acids Res. 10:6487-500 (1982).

As used herein, the phrase “specific binding” or “selective binding” refers to a binding reaction which is determinative of the presence of the mutant Trx in a heterogeneous population of proteins and other biologics. Thus, under designated conditions, the antibodies or fragments thereof of the present invention bind to a mutant Trx (e.g., the C32S and/or the C35S mutant), fragment, or variant thereof and do not bind in a significant amount to other proteins (e.g., wildtype Trx), present in the subject. The absence of binding in the present invention is considered to be binding that is less than 1.5 times background (i.e., the level of non-specific binding or slightly above non-specific binding levels). Selective binding to an antibody under such conditions may require an antibody that is selected for its specificity for a particular protein, variant, or fragment. Thus, as one embodiment, the antibody selectively binds a polypeptide comprising the amino acid sequence of SEQ ID NO:2, or SEQ ID NO:3 or both.

A variety of immunoassay formats may be used to select antibodies that selectively bind with a particular protein, variant, or fragment. For example, solid-phase ELISA immunoassays are routinely used to select antibodies selectively immunoreactive with a protein, variant, or fragment thereof. See Harlow and Lane. Antibodies, A Laboratory Manual. Cold Spring Harbor Publications, New York, (1988), for a description of immunoassay formats and conditions that could be used to determine selective binding. The binding affinity of a monoclonal antibody can, for example, be determined by the Scatchard analysis of Munson et al., Anal. Biochem., 107:220 (1980).

The invention also provides an antibody reagent kit comprising the antibody or fragment thereof of the invention and reagents for detecting binding of the antibody or fragment thereof to a ligand. The kit can further comprise containers containing the antibody or fragment thereof of the invention and containers containing the reagents. Preferably the ligand is a Trx mutant, variant, or fragment thereof. The kit can include an antibody bound to a substrate, a secondary antibody reactive with the antigen and a reagent for detecting a reaction of the secondary antibody with the antigen. Such a kit can be an ELISA kit and can comprise the substrate, primary and secondary antibodies when appropriate, and any other necessary reagents such as detectable moieties, enzyme substrates and color reagents as described above. The diagnostic kit can, alternatively, be an immunoblot kit generally comprising the components and reagents described herein. Alternatively, the kit could be a radioimmunoassay kit, a Western blot assay kit, an immunohistological assay kit, an immunocytochemical assay kit, a dot blot assay kit, a fluorescence polarization assay kit, a scintillation proximity assay kit, a homogeneous time resolved fluorescence assay kit, or a BIAcore analysis kit.

As used throughout, methods of detecting an Trx mutant or antigen/antibody complexes, including complexes comprising a mutant Trx and optionally the antibody of the present invention, can comprise an ELISA (competition or sandwich), a radioimmunoassay, a Western blot assay, an immunohistological assay, an immunocytochemical assay, a dot blot assay, a fluorescence polarization assay (Jolley (1981); Jiskoot et al (1991); Seethala et al. (1998); Bicamumpaka et al. (1998)), a scintillation proximity assay (Amersham Life Science (1995) Proximity News. Issue 17; Amersham Life Science (1995) Proximity News. Issue 18; Park et al. (1999)), a homogeneous time-resolved fluorescence assay (Park et al. (1999); Stenroos et al. (1988); Morrison, 1988)), or a BIAcore analysis (Fagerstam et al. (1992) Chromatography 597:397-410). Preferably, the antigen/antibody complex is detectably tagged either directly or indirectly. Any desired tag can be utilized, such as a fluorescent tag, a radiolabel, a magnetic tag, or an enzymatic reaction product.

Optionally, the antibody or fragment is a humanized antibody or a fully human antibody. For example, the antibodies can also be generated in other species and “humanized” for administration to humans. Alternatively, fully human antibodies can also be made by immunizing a mouse or other species capable of making a fully human antibody (e.g., mice genetically modified to produce human antibodies), screening clones that bind mutant Trx. See, e.g., Lonberg and Huszar (1995) Human antibodies from transgenic mice, Int. Rev. Immunol. 13:65-93, which is incorporated herein by reference in its entirety for methods of producing fully human antibodies. As used herein, the term “humanized” and “fully human” in relation to antibodies, relate to any antibody which is expected to elicit a therapeutically tolerable weak immunogenic response in a human subject.

Humanized forms of non-human (e.g., murine) antibodies are chimeric immunoglobulins, immunoglobulin chains or fragments thereof (such as Fv, Fab, Fab′ , F(ab′)₂, or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all or at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-327 (1988); and Presta, Curr. Op. Struct. Biol., 2:593-596 (1992)).

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important in order to decrease antigenicity. According to the “best-fit” method, the sequence of the variable domain of a rodent antibody is screened against the entire library of known human variable domain sequences. The human sequence which is closest to that of the rodent is then accepted as the human framework (FR) for the humanized antibody (Sims et al., J. Immunol., 151:2296 (1993) and Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding (see, WO 94/04679 published 3 Mar. 1994).

Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge (see, e.g., Jakobovits et al., Proc. Natl. Acad. Sci. USA, 90:2551-255 (1993); Jakobovits et al., Nature, 362:255-258 (1993); Bruggemann et al, Year in Immunol, 7:33 (1993)). Human antibodies can also be produced in phage display libraries (Hoogenboom et al., J. Mol. Biol., 227:381 (1991); Marks et al., J. Mol. Biol., 222:581 (1991)). The techniques of Cote et al. and Boemer et al. are also available for the preparation of human monoclonal antibodies (Cole et al., Monoclonal Antibodies and Cancer Therapy, Alan R. Liss, p. 77 (1985); Boemer et al., J Immunol, 147(1):86-95 (1991)).

In one embodiment, the antibody or fragment thereof is a single chain antibody. In another embodiment, the antibody or fragment is labeled. Optionally the antibody or fragment is conjugated or fused with a toxin or fragment thereof. Examples of the toxin or toxin moiety include diphtheria, ricin, and modifications thereof.

Nucleic Acids

Also provided is an isolated nucleic acid that encodes the mutant Trx of the invention. The nucleic acid can be single or double stranded and can be RNA or DNA. More specifically, the invention provides an isolated nucleic acid, comprising a nucleotide sequence that encodes SEQ ID NO:2 or SEQ ID NO:3, optionally with one or more conservative amino acid substitutions. Optionally the nucleic acid further encodes a signal sequence. The isolated nucleic acid optionally encodes the sequences with 80, 85, 90, or 95% identity. More specifically, the invention provides an isolated nucleic acid, comprising a nucleotide sequence of SEQ ID NO:5 or SEQ ID NO:6. Optionally, the isolated nucleic acid can further include bases that encode a signal sequence.

The invention also provides an isolated nucleic acid comprising a sequence that hybridizes under stringent conditions to a hybridization probe, wherein the hybridization probe comprises the nucleotide sequence of SEQ ID NO:5 or SEQ ID NO:6 or the complement of either sequence. Further provided is a single stranded nucleic acid that hybridizes under stringent conditions to a nucleic acid having the sequence of SEQ ID NO:5, SEQ ID NO:6, SEQ ID NO:13, SEQ ID NO:14. In a preferred embodiment, the isolated nucleic acid does not hybridize with the nucleic acid sequence encoding SEQ ID NO:1 or with a nucleic acid sequence of SEQ ID NO:4. In another preferred embodiment the nucleic acid does not hybridize with the nucleic acid encoding SEQ ID NO:9 or with the nucleic acid of SEQ ID NO:12.

By “hybridizing under stringent conditions” or “hybridizing under highly stringent conditions” is meant that the hybridizing portion of the hybridizing nucleic acid, typically comprising at least 15 (e.g., 20, 25, 30, or 50 nucleotides), hybridizes to all or a portion of the provided nucleotide sequence under stringent conditions. The term “hybridization” typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize. Generally, the hybridizing portion of the hybridizing nucleic acid is at least 80%, for example, at least 90%, 95%, or 98%, identical to the sequence of or a portion of a nucleic acid encoding an Trx of the invention, or its complement. Hybridizing nucleic acids of the invention can be used, for example, as a cloning probe, a primer (e.g., for PCR), a diagnostic probe, or an antisense probe. Hybridization of the oligonucleotide probe to a nucleic acid sample typically is performed under stringent conditions. Nucleic acid duplex or hybrid stability is expressed as the melting temperature or Tm, which is the temperature at which a probe dissociates from a target DNA. This melting temperature is used to define the required stringency conditions. If sequences are to be identified that are related and substantially identical to the probe, rather than identical, then it is useful to first establish the lowest temperature at which only homologous hybridization occurs with aparticular concentration of salt (e.g., SSC or SSPE). Assuming that a 1% mismatch results in a 1° C. decrease in the Tm, the temperature of the final wash in the hybridization reaction is decreased accordingly (for example, if sequence having >95% identity with the probe are sought, the final wash temperature is decreased by 5 ° C.). In practice, the change in Tm can be between 0.5° C. and 1.5° C. per 1% mismatch. Stringent conditions involve hybridizing at 68° C. in 5× SSC/5× Denhardt's solution/1.0% SDS, and washing in 0.233 SSC/0.1% SDS at room temperature. Moderately stringent conditions include washing in 3× SSC at 42° C. The parameters of salt concentration and temperature can be varied to achieve the optimal level of identity between the probe and the target nucleic acid. Additional guidance regarding such conditions is readily available in the art, for example, in Sambrook et al., 1989, Molecular Cloning, A Laboratory Manual, Cold Spring Harbor Press, NY; and Ausubel et al. (eds.), 1995, Current Protocols in Molecular Biology, (John Wiley & Sons, NY) at Unit 2.10.

The nucleic acids of the present invention are optionally labeled, directly or indirectly. Such labeled nucleic acids are useful in various diagnostic techniques including for example, in situ hybridization, FISH, in situ PCR, and PRINS.

Other nucleic acids of the invention are useful as anti-sense oligos, RNA interference (RNAI) or small interfering RNA (SiRNA), or other nucleic acids designed to decrease specific expression. For example, an siRNA for non-mitochondrial Trx is the doubled stranded RNA sequence corresponding to SEQ ID NO:22. An example of an siRNA for mitochondiral Trx is the double stranded RNA sequence corresponding to the sequence of SEQ ID NO:23, and an example of an siRNA for ASK1 is double stranded RNA sequence corresponding to SEQ ID NO:24. Optionally the 3′ end of the siRNA sequences further comprises one, two, or more dioxythymidine residues.

Nucleic acids useful in RNAi methods include those directed toward blocking Trx expression (e.g., the sequences of SEQ ID NO:25, SEQ ID NO:26, SEQ ID NO:27, SEQ ID NO:28, SEQ ID NO:29, SEQ ID NO:30, SEQ ID NO:31, and SEQ ID NO:32 or the complements of the listed sequences or sequences that hybridize under stringent conditions to the listed sequences). Other nucleic acids useful in RNAi methods include those directed toward blocking mitochondrial Trx expression and include those sequences of SEQ ID NO:33, SEQ ID NO:34, SEQ ID NO:35, SEQ ID NO:36, SEQ ID NO:37, SEQ ID NO:38 or the complements ofhte listed sequences or sequences that hybridize under stringent condition to the listed sequences. Examples of nucleic acids useful in blocking expression of ASK1 include for example the sequences of SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58 or their complements or sequences that hydridize understringent condition to the listed sequences.

Vectors, Cells, and Methods of Using

Also provided is an expression vector comprising a nucleic acid of the invention, wherein the nucleic acid is operably linked to an expression control sequence. A wide variety of expression system/regulatory sequence combinations may be employed in expressing the disclosed. Such useful regulatory sequences include, for exarnple, the early or late promoters of SV40, CMV, vaccinia, polyoma or adenovirus, the lac system, the trp system, the TAC system, the TRC system, the LTR system, the major operator and promoter regions of phage lambda, the control regions of fd coat protein, the promoter for 3-phosphoglycerate kinase or other glycolytic enzymes, the promoters of acid phosphatase (for example, PhoS), the AOX 1 promoter of methylotrophic yeast, the promoters of the yeast a-mating factors, and other sequences known to control the expression of genes of prokaryotic or eukaryotic cells or their viruses, and various combinations thereof.

Such an expression vector can be designed to be expressed by eukaryotic cells or prokaryotic cells. The vectors of the present invention thus provide DNA molecules which are capable of integration into a prokaryotic or eukaryotic chromosome and expression. The inserted genes in viral and retroviral vectors usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements. It has been shown that all specific regulatory elements can be cloned and used to construct expression vectors that are selectively expressed in specific cell types. For example, the glial fibrillary acetic protein (GFAP) promoter has been used to selectively express genes in cells of glial origin. Expression vectors used in eukaryotic host cells (e.g., yeast, fungi, insect, plant, animal, human or nucleated cells) may also contain sequences necessary for the termination of transcription which may affect mRNA expression. These regions are transcribed as polyadenylated segments in the untranslated portion of the MRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like MRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

The invention further provides transfer vectors, which include any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)). As used herein, plasmid or viral vectors are agents that transport the disclosed nucleic acids into the cell without degradation and include a promoter yielding expression of the gene in the cells into which it is delivered. In some embodiments the mutant Trxs are derived from either a virus or a retrovirus. Viral vectors include, for example, Adenovirus, Adeno-associated virus, Herpes virus, Vaccinia virus, Polio virus, HIV, neuronal trophic virus, Sindbis and other RNA viruses, including these viruses with the HIV backbone. Also preferred are any viral families that share the properties of these viruses that make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines that have been engineered to express the gene products of the early genes in trans.

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference. A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules that are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of one to many genes depending on the size of each S transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line that has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

The construction of replication-defective adenoviruses has been described (Berkner et al., J Virology 61:1213-1220 (1987); Massie et al., Mol Cell Biol 6:2872-2883 (1986); Haj-Ahmad et al., J Virology 57:267-274 (1986); Davidson et al., J Virology 61:1226-1239 (1987); Zhang, Generation and identification of recombinant adenovirus by liposome-mediated transfection and PCR analysis, BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J Clin Invest 92:1580-1586 (1993); Kirshenbaum, J Clin Invest 92:381-387 (1993); Roessler, J Clin Invest 92:1085-1092 (1993); Moullier, Nature Genetics 4:154-159 (1993); La Salle, Science 259:988-990 (1993); Gomez-Foix, J Biol Chem 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur J Neuroscience 5:1287-1291 (1993); and Ragot, J Gen Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J Virology 12:386-396 (1973); Svensson and Persson, J Virology 55:442-449 (1985); Seth, et al., J Virol 51:650-655 (1984); Seth, et al., Mol Cell Biol 4:1528-1533 (1984); Varga et al., J Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus. U.S. Pat. No. 6,261,834 is herein incorporated by reference for material related to the AAV vector.

Molecular genetic experiments with large human herpesviruses have provided a means whereby large heterologous DNA fragments can be cloned, propagated and established in cells permissive for infection with herpesviruses (Sun et al., Nature genetics 8: 3341, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes. Other useful systems include, for example, replicating and host-restricted non-replicating vaccinia virus vectors.

The invention also provides an isolated cell comprising a vector of the invention. The isolated cell can be either a eukaryotic or prokaryotic cell, such as strains of E. coli, Pseudomonas, Bacillus, Streptomyces; fungi such as yeasts (Saccharomyces, and methylotrophic yeast such as Pichia, Candida, Hansenula, and Torulopsis); and animal cells, such as CHO, R1.1, B-W and LM cells, African Green Monkey kidney cells (for example, COS 1, COS 7, BSC1, BSC40, and BMT10), insect cells (for example, Sf9), and human cells and plant cells in tissue culture.

Also provided is a method of making a mutant Trx, or a fragment or variant thereof comprising culturing a cell comprising a vector of the invention under conditions permitting expression of the mutant Trx. The method comprises culturing a cell comprising an exogeneous nucleic acid that encodes the Trx, fragment, or variant, wherein the exogeneous nucleic acid is operably linked to an expression control sequence, and wherein the culture conditions permit expression of the Trx, fragment, or variant under the control of the expression control sequence; harvesting the medium from the cultured cells, and isolating the mutant Trx, fragment, or varinat from the cell or culture medium. Optionally the exogenous nucleic acid is the nucleotide sequence of SEQ ID NO:5, SEQ ID NO:6, SEQ ID NO: 10, SEQ ID NO:11, SEQ ID NO:18, SEQ ID NO:19. Optionally, the exogenous nucleic acid further comprises a nucleotide sequence that encodes a signal sequence. In the recombinant methods, the cell can be any known host cell, including for example, a prokaryotic or eukaryotic cell. The nucleic acids that are delivered to cells, generally in a plasmid or other vector, typically contain expression controlling systems. For example, the inserted genes in viral and retroviral systems usually contain promoters, and/or enhancers to help control the expression of the desired gene product.

Those skilled in the art of molecular biology will understand that a wide variety of expression systems may be used to produce recombinant Trx polypeptides (as well as fragments, fusion proteins, and amino acid sequence variants with therapeutic activity) for use in the methods of the invention. Thus, Trx may be produced using prokaryotic host cells (e.g., Escherichia coli) or eukaryotic host cells (e.g., Saccharomyces cerevisiae, insect cells such as Sf9 cells, or mammalian cells such as CHO cells, COS-1, NIH 3T3, or HeLa cells). These cells are commercially 20. available from, for example, the American Type Culture Collection, Rockville, Md. (see also F. Ausubel et al., Current Protocols in Molecular Biology, John Wiley & Sons, New York, N.Y., 1998). The method of transformation and the choice of expression vector will depend on the host system selected. Transformation and transfection methods are described, e.g., in Ausubel et al., supra, and expression vectors may be chosen from the numerous examples known in the art.

A nucleic acid sequence encoding a mutant Trx is introduced into a plasmid or other vector, which is then used to transform living cells. Constructs in which a cDNA containing the entire Trx coding sequence, a fragment of the Trx coding sequence, amino acid variations of the Trx coding sequence, or fusion proteins of the aforementioned, inserted in the correct orientation into an expression plasmid, may be used for protein expression. In some cases, for example, it may be desirable to express the Trx coding sequence under the control of an inducible or tissue-specific promoter.

Eukaryotic expression systems permit appropriate post-translational modifications to expressed proteins. Thus, eukaryotic, and more preferably mammalian expression systems, allow glycosylations patterns comparable to naturally expressed Trx. Transient transfection of a eukaryotic expression plasmid allows the transient production of Trx by a transfected host cell. Trx may also be produced by a stably-transfected mammalian cell line. A number of vectors suitable for stable transfection of mammalian cells are available to the public (e.g., see Pouwels et al., Cloning Vectors: A Laboratory Manual, 1985, Supp. 1987), as are methods for constructing such cell lines (see e.g., F. Ausubel et al., Current Protocols in Molecular Biology, John Wiley & Sons, New York, N.Y., 1998). Another preferred eukaryotic expression system is the baculovirus system using, for example, the vector pBacPAK9, which is available from Clontech (Palo Alto, Calif.). If desired, this system may be used in conjunction with other protein expression techniques, for example, the myc tag approach described by Evan et al. (Mol. Cell Biol. 5:3610-3616, 1985) or analogous tagging approaches, e.g., using a hemagluttinin (HA) tag.

Once the recombinant protein is expressed, it can be isolated from the expressing cells by cell lysis followed by protein purification techniques such as affinity chromatography. In this example, an antibody that specifically binds to mutant Trx, which may be produced by methods that are well-known in the art, can be attached to a colunm and used to isolate mutant Trx. Once isolated, the recombinant protein can, if desired, be purified further, e.g., by high performance liquid chromatography (HPLC; e.g., see Fisher, Laboratory Techniques In Biochemistry And Molecular Biology, Work and Burdon, Eds., Elsevier, 1980).

Methods of Use

The present invention also relates to a method of decreasing inflammation, apoptosis, endothelial cell dysfunction, cardiomyocyte contractile dysfimction, or insulin resistance in a target tissue. The present invention also relates to methods of to increasing or decreasing Trx-induced protein ubiquitination and degradation.

More specifically, the invention can relate to decreasing inflammation induced by a reactive oxygen species or cytokine-induced inflammation or any other inflammatory process mediated by a redox-regulated pathway. The steps of the methods of decreasing inflammation, apoptosis endothelial cell dysfunction, cardiomyocyte contractile dysfunction, and insulin resistance comprise contacting the target tissue with the mutant thioredoxin molecule or a small molecule that blocks a thiol group of the Trx catalytic site. Decreasing Trx-induced protein degradation could similarly be accomplished using the mutant thioredoxin molecule or a small molecule that blocks a thiol group of the Trx catalytic site.

By “decreasing inflammation” is meant lessening a clinical characteristic (e.g., pain, swelling, stiffness, warmth) or a laboratory index of inflammation (e.g., activated macrophages, vascular permeability, etc.) in the preparation or subject as compared to a control preparation or subject lacking the mutant Trx. Decrease includes complete elimination of the inflammation, a substantial lessening, or a minor lessening. Thus, a decrease in inflammation can be marked by 10, 20, 30, 40, 50, 60, 70, 80, 90, or 100% decrease in at least one clinical characteristic or laboratory index.

By decreasing “apoptosis” means a lessening in the number of apoptotic cells or the number of cells showing indicators of apoptosis in the target tissue as compared to the number in a control preparation or subject lacking the mutant Trx.

By a decrease in insulin resistance is meant a lessening in the insulin resistance of at least one cell type in the target tissue (e.g., pancreatic islet cells, and more particularly β cell) as compared to the cell in target tissues in the absence of mutant Trx. The net effect therefore is to increase insulin sensitivity. Insulin resistance can be measured by a diminished blood glucose response to insulin, e.g., such that blood glucose levels are elevated in a resistant state.

By decrease in cardiomyocyte contractile dysfunction is meant a lessening in the impaired cardiac function in response to mediators like proinflammatory mediators, reactive oxygen species, and oxidative stress as compared to cardiomyocytes exposed toth e same mediators, reactive oxygen species, or stress in the absence of the mutant Trx. Impaired cardic function can be evaluated by one skilled in the art by such analyses as contractility of cardiomycytes, cardiac output, levels of myofibrillar ATPase, actimyosien responses, etc.

By “target tissue” is meant the biological tissue to which the mutant thioredoxin is directed. Target tissues, include for example, epithelial, connective, muscular, and neural tissues. Target tissues further include cutaneous, muscular, vascular, and cardiac tissues, as well as bone, cartilage, digestive, respiratory, urinary and reproductive tissues, or any other tissue know to have or suspected of having inflammation. Target tissues can also include certain cancerous tissues such as tumors.

By “contacting” or “administration” is meant either “in vivo” or “in vitro” contact between cells of the target tissue and the therapeutic agent. The contacting step can occur directly by administering the mutant TRX or indirectly by administering a precursor or prodrug thereof or by expression of a nucleic acid that encodes the mutant Trx. Thus, in one embodiment, cells in vivo or in vitro are made to express and secrete the mutant Trx so that the expressed mutant Trx contacts the target tissue.

As described in the methods herein, the mutant Trx could act as a competitive inhibitor of wild-typeTrx, wherein the mutant Trx, unlike the wildtype Trx, is resistant to cytokine and ROS-induced effects. By binding to ASK1, the mutant Trx blocks the wild-type sensitivity to cytokines and ROS. A similar effect is achieved by contacting the wild-type thioredoxin molecule with a small molecule that blocks a thiol group of the wild-type. Thus, cytokine-induced or ROS-induced inflammation, insulin resistance, or apoptosis in target tissues can similarly be decreased by small molecules that block a thiol group in wildtype Trx. Other mutants useful in the present methods include mutations at sites in addition to C32 and C35. C69 may be one of the sites. The S-nitrosylation of a SH-group by NO is important for regulation of cellular signaling, especially in endothelial cell function. The majority of S-nitrothiols (95%) is associated with proteins including signaling molecules, proteases, channels and transcription factors. Trx is S-nitrosylated at C69 site and the nitrosylation of Trx is important for its anti-apoptotic function. C69 is involved in ASK1 binding and mutations at that residue inhibit apoptosis. S-nitrosylation is blocked by small molecules or other agents, such as NO synthase Inhibitors like NG-monomethyl-L-arginine (L-NMMA), N-nitro-L-arginine (L-NAG) and N-nitro-L-arginine methyl ester (L-NAME). Also, S-nitrosylation at C69 is critical for Trx-induced ubiquitination and degradation of other proteins, such as ASK1. Thus mutants that comprise or further comprise a substitution or deletion mutation at C69 or a comparable residue are especially useful in diseases in which apoptosis or other NO mediated event is desired. Such a mutant would have the effect of decreasing the ability to be nitrosylated or ASK1-binding leading to increased apoptosis. Thus, these mutants can enhance apoptosis and decrease angiogenesis. As a result, these mutants have therapeutic uses.

The present invention thus relates to a method of treating subjects with or at risk of atherosclerosis, diabetes, apoptotic disease, mitochondrial dysfunction disease, or cardiac dysfunction comprising administering to the subject a therapeutic agent of the invention. For example, an agent that inactivates endothelial cell dysfunction mediated by redox-regulated pathways is useful in a subject with atherosclerosis. “Endothelial cell dysfunction,” as used herein, refers to inflammatory or atherosclerotic processes including disruption in normal coagulation, thrombosis, relaxation, or survival.

Mitochondrial dysfunction diseases include Kearns-Sayre syndrome, myoclonus epilepsy with ragged-red fibers (MERRF), and mitochondrial encephalomyopathy lactic acidosis and stroke-like episodes (MELAS), and others known in the art.

An agent that inactivates a redox-regulated pathway that mediates insulin resistance is useful in the treatment of diabetes, particularly Type 2 diabetes. Similarly an agent that inactivates a redox pathway-mediated apoptosis is useful in the treatment of an apoptotic disease (e.g., a neurodegenerative disease such as Alzheimer's Disease, Parkinson's disease, Huntington's disease, amyotrophic lateral sclerosis, multiple sclerosis, or peripheral neuropathy).

The therapeutic agent useful in the methods of treating atherosclerosis, diabetes, cardiac dysfunction, mitochonrial dysfimction disease, diseases characterized by insufficient angiogenesis, or an apoptotic disease can include a mutant Trx described herein or a small molecule that blocks the thiol group of wild-type Trx. As further described herein the administration of the Trx or the small molecule can be performed indirectly by providing a precursor, prodrug, or by providing a nucleic acid encoding a mutant Trx or small molecule, whereby the mutant Trx or small molecule is expressed and secreted by cells of the subject.

The invention also provides methods of treating angiogenesis dependent diseases. By “angiogenesis dependent diseases” is meant diseases characterized by excessive angiogenesis (cancer, diabetic blindness, age-related macular degeneration, rheumatoid arthritis, psoriasis, atherosclerosis and other conditions recognized by those skilled in the art). In contrast to the treatment of atherosclerosis, diabetes, and apoptotic diseases, for example, as described above, agents useful in the treatment of angiogenesis dependent diseases comprise administering to a subject in need of an agent that blocks inactivation of apoptosis by thioredoxin, mediated by a redox-regulated pathway. One agent useful in blocking the inactivation of apoptosis by thioredoxin is an antibody of the invention, wherein the antibody blocks Trx binding to ASK1. Since the binding domain of Trx interacting with ASK1 has been mapped, the peptide corresponding to the domain can be used as an immunogen to generate a blocking antibody.

Other agents useful in blocking inactivation of apoptosis by thioredoxin, mediated by a redox-regulated pathway include anti-sense oligos, RNA interference (RNAi) or small interfering RNA (SiRNA). Methods of RNAi and SiRNA are described in detail in Hannon et al. (2002), RNA Interference, Nature 418:244-250; Brummelkamp et al. (2002), A System for Stable Expression of Short Interfering RNAs in Mammalian Cells, Science 296:550-508; Paul et al. (2002), Effective expression of small interfering RNA in human cells, Nature Biotechnology 20: 505-508, which are each incorporated by reference in their entirety for methods of RNAi and SiRNA and for designing and testing various oligos useful therein. Such methods could be directed at blocking either non-mitochondrial or mitochondrial Trx. In contrast, methods of blocking ASK1 expression using similar methods would be useful in treating disorders involving excessive apoptosis (e.g., atherosclerosis, diabetes, and apoptotic diseases).

The invention also provides a method of treating diseases associated with insufficient angiogenesis. Diseases associated with insufficient angiogenesis include, but are not limited to, coronary artery disease, stroke, and delayed wound healing. Therapeutic agents of the invention useful in the methods of treating diseases associated with insufficient angiogensis include a mutant Trx described herein or a small molecule that blocks the thiol group of wild-type Trx. As further described herein the administration of the Trx or the small molecule can be performed indirectly by providing a precursor, prodrug, or by providing a nucleic acid encoding a mutant Trx or small molecule, whereby the mutant Trx or small molecule is expressed and secreted by cells of the subject.

Typically, the reagent or composition is administered to the subject transdermally (e.g., by a transdermal patch or a topically applied cream, ointment, or the like), orally (such as by tablets, capsules, granules, powders and syrups), parenterally (such as by injection, dropping injection and suppositories), subcutaneously, intrapulmonarily, transmucosally, intraperitoneally, intrauterinely, sublingually, intrathecally, intramuscularly, intraarticularly, etc. using conventional methods. In addition, the reagent or composition can be administered via injectable depot routes such as by using 1-, 3-, or 6-month depot injectable or biodegrable materials and methods.

Regardless of the route of administration, the amount of the reagent administered or the schedule for administration will vary among individuals based on age, size, weight, condition to be treated, mode of administration, and the severity of the condition. One skilled in the art will realize that dosages are best optimized by the practicing physician and methods for determining dosage are described, for example in Remington's Pharmaceutical Science, latest edition. For example, a typical dose of the mutant Trx used alone might range from about 10 μg/kg to up to 1000 μg/kg of body weight or more per day, and preferably 100 μg/kg to up to 500 μg/kg, depending on the factors mentioned above.

The nucleic acids of the invention (e.g., cDNAs, genes, anti-sense oligos, RNAi and SiRNA) can be delivered to cells in a variety of ways. For example, if the nucleic acid of this invention is delivered to the cells of a subject in an adenovirus vector, the dosage for administration of adenovirus to humans can range from about 10⁷ to 10⁹ plaque forming units (pfu) per injection, but can be as high as 1012 pfu per injection. Ideally, a subject will receive a single injection. If additional injections are necessary, they can be repeated at six-month intervals for an indefinite period and/or until the efficacy of the treatment has been established. As set forth herein, the efficacy of treatment can be determined by evaluating the clinical parameters. The exact amount of the nucleic acid or vector required will vary as described above. Thus, it is not possible to specify an exact amount for every nucleic acid or vector. An appropriate amount can be determined by one of ordinary skill in the art using only routine experimentation given the teachings herein.

The effectiveness of the method of treatment can be assessed by monitoring the patient for known signs or symptoms of the conditions being treated. For example, in the treatment of diabetes, the stabilization of blood glucose levels after feeding or fasting would indicate successful treatment. In the treatment of arthritis, for example, a decrease in the amount ofjoint inflammation would indicate successful treatment. In the treatment of cardiac dysfunction, for example, an increase in cardiac output would indicate successful treatment. Thus, by “therapeutically effective” is meant an amount that provides the desired treatment effect.

Depending on the intended mode of administration, the therapeutic agent can be in pharmaceutical compositions in the form of solid, semi-solid or liquid dosage forms, such as, for example, tablets, suppositories, pills, capsules, powders, liquids, or suspensions, preferably in unit dosage form suitable for single administration of a precise dosage. The compositions will include an effective amount of the selected substrate in combination with a pharmaceutically acceptable carrier and, in addition, may include other medicinal agents, pharmaceutical agents, carriers, or diluents. By “pharmaceutically acceptable” is meant a material that is not biologically or otherwise undesirable, which can be administered to an individual along with the selected substrate without causing significant undesirable biological effects or interacting in a deleterious manner with any of the other components of the pharmaceutical composition in which it is contained.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example, sugars, sodium chloride, and the like. Prolonged absorption of the injectable pharmaceutical form can be brought about by the use of agents delaying absorption, for example, aluminum monostearate and gelatin.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others well known in the art. They may contain opacifying agents, and can also be of such composition that they release the active compound or compounds in a certain part of the intestinal tract in a delayed manner. Examples of embedding compositions which can be used are polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl alcohol, benzyl benzoate, propyleneglycol, 1,3-butyleneglycol, dimethylformamide, oils, in particular, cottonseed oil, groundnut oil, corn gerM oil, olive oil, castor oil and sesame oil, glycerol, tetrahydrofurfuryl alcohol, polyethyleneglycols and fatty acid esters of sorbitan or mixtures of these substances, and the like.

Besides such inert diluents, the composition can also include adjuvants, such as wetting agents, emulsifying and suspending agents, sweetening, flavoring, and perfuming agents.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are preferably suppositories which can be prepared by mixing the compounds of the present invention with suitable non-irritating excipients or carriers such as cocoa butter, polyethyleneglycol or a suppository wax, which are solid at ordinary temperatures but liquid at body temperature and therefore, melt in the rectum or vaginal cavity and release the active component.

Dosage forms for topical administration of a compound of this invention include ointments, powders, sprays, and inhalants. The active component is admixed under sterile conditions with a physiologically acceptable carrier and any preservatives, buffers, or propellants as may be required. Ophthalmic formulations, ointments, powders, and solutions are also contemplated as being within the scope of this invention.

The term “pharmaceutically acceptable salts, esters, amides, and prodrugs” as used herein refers to those carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, cornmensurate with a reasonable benefit/risk ratio, and effective for their intended use, as well as the zwitterionic forms, where possible, of the compounds of the invention. The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate and laurylsulphonate salts, and the like. These may include cations based on the alkali and alkaline earth metals, such as sodium, lithium, potassium, calcium, magnesium, and the like, as well as non-toxic ammonium, quaternary ammonium and amine cations including, but not limited to ammonium, tetramethylammonium, tetraethylammonium, methylamine, dimethylamine, trimethylamine, triethylamine, ethylamine, and the like. (See, for example, S. M. Barge et al., “Pharmaceutical Salts,” J. Pham. Sci., 1977, 66:1-19 which is incorporated herein by reference.)

The term “prodrug” refers to compounds that are rapidly transformed in vivo to yield the parent compounds of the above formula, for example, by hydrolysis in blood. A thorough discussion is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

Diagnostic Methods

Genetic mutations in the gene encoding Trx can result in mutations that inactivate the induction of apoptosis by redox-regulated pathways and cause a disease state. For example, a genetic mutation that disrupts the C32 and C35 sites of Trx molecules results in a decrease in cell death or an increase in cell proliferation. A genetic mutation that disrupts S-nitrosylation by NO of a SH-group of the C69 residue, for example, could increase cell death and decrease cell proliferation.

In one embodiment, the invention relates to a method of diagnosing an angiogenesis dependent disease in a subject or of identifying a subject at risk for developing the angiogenesis dependent disease, comprising detecting, in a biological sample of the subject, levels of reduced thioredoxin, wherein the angiogenesis dependent disease is indicated by an elevated level of reduced thioredoxin as compared to control levels (i.e., levels in subjects without an angiogenesis dependent disease). Control samples may show a higher level of oxidized Trx, thus, the biological sample to be tested may conversely be screened for a lower level of oxidized Trx as compared to control.

In another embodiment, the invention relates to a method of diagnosing an apoptotic disease in a subject or of identifying a subject at risk for developing the apoptotic disease, comprising detecting, in a biological sample of the subject, levels of oxidized thioredoxin, wherein the apoptotic disease is indicated by an elevated level of oxidized thioredoxin as compared to control levels. Control samples may show a higher level of reduced Trx, thus, the biological sample to be tested may conversely be screened for a lower level of reduced Trx as compared to control.

Mutations at the C69 or comparable residue could be diagnostic of an apoptotic disease or a cardiomyocyte contractile dysfunction disease or a predisposition for such diseases. Elevated levels of mutant Trx in these tissues would thus be indicative of the aforementioned diseases.

Comparable mutations in mitochondrial Trx can similar be predictive of mitochondrial dysfunction diseases.

By “biological sample,” as used throughout, refers to a sample from any organism. The sample can be, but is not limited to, peripheral blood, plasma, urine, saliva, gastric secretion, feces, bone marrow specimens, primary tumors, embedded tissue sections, frozen tissue sections, cell preparations, cytological preparations, exfoliate samples (e.g., sputum), fine needle aspirations, amnion cells, fresh tissue, dry tissue, and cultured cells or tissue. It is further contemplated that the biological sample of this invention can also be whole cells or cell organelles (e.g., nuclei). The sample can be unfixed or fixed according to standard protocols widely available in the art and can also be embedded in a suitable medium for preparation of the sample. For example, the sample can be embedded in paraffin or other suitable medium (e.g., epoxy or acrylamide) to facilitate preparation of the biological specimen for the detection methods of this invention.

Detection of reduced or oxidized forms of Trx can be performed using a variety of methods known in the art and described herein.

The invention also provides a method of screening a subject for a genetic risk of an angiogenesis dependent disease or other diseases, comprising detecting a nucleic acid that encodes a mutant thioredoxin molecule. More specifically, the genetic analysis is performed to detect a nucleic acid encoding a mutant thioredoxin molecule that is resistant to the oxidizing effects of cytokines or reactive oxygen species as described herein. Thus, a nucleic acid encoding the C32 or C35 or C69 mutant is detected.

Mutations in Trx or nucleic acids encoding same can also be predictive of diseases such as cystic fibrosis, immune-deficiency/inflammatory diseases and muscle waste.

The following examples are set forth below to illustrate the methods and results according to the present invention. These examples are not intended to be inclusive of all aspects of the present invention, but rather to illustrate representative methods and results. These examples are not intended to exclude equivalents and variations of the present invention which are apparent to one skilled in the art

### EXAMPLE 1

Thioredoxin Promotion of ASK1 Ubiqutination and Degradation and Thioredoxin Inhibition of ASK1-Mediated Apoptosis

Materials and Methods

Plasmids Mammalian expression plasmids for poly-Ub were provided by Dr. Dirk Bohmann (University of Rochester) (Liu et al., “Activation of apoptosis signal-regulating kinase 1 (ASK1) by tumor necrosis factor receptor-associated factor 2 requires prior dissociation of the ASK1 inhibitor thioredoxin.” Mol Cell Biol 20:2198-208 (2000)). Human thioredoxin cDNA was cloned by RT-PCR using total RNA from HUVEC with a pair of primers: the sense primer was 5′AAGCTT ATGGTGAAGCAGATCGAG3′ (SEQ ID NO:7) (the Hind III site is underlined) and the antisense primer was 5′CTCGAG TTAGACTAATTCATTAAT (SEQ ID NO:8) (the Xho I site is underlined). The cDNA was confirmed by DNA sequencing. Mutations of C32S and C35S in Trx were introduced by recombinant PCR (Liu Y et al. Laminar flow inhibits TNF-induced ASK1 activation by preventing dissociation of ASK1 from its inhibitor 14-3-3. J Clin Invest 107:917-23 (2001)) and confirmed by DNA sequencing. Expression plasmids for Flag-tagged Trx were constructed into the Flag-vector (Liu Y et al. Laminar flow inhibits TNF-induced ASK1 activation by preventing dissociation of ASK1 from its inhibitor 14-3-3. J Clin Invest. 107:917-23 (2001)).

Cells and cytokines Bovine aorta endothelial cells (BAEC) and human umbilical vein EC (HUVEC) were purchased from Clonetics (San Diego, Calif.). Human rTNF was from R&D Systems Minneapolis, Minn.) and used at 10 ng/ml.

Transfection Transfection of EC was performed by Lipofectamine2000 according to manufacturer's protocol (Gibco). Cells were cultured at 90% confluence in 6-well plates and were transfected with total 4 μg plasmid constructs as indicated. Cells were harvested at 36-48 h post-transfection and cell lysates were used for protein assays.

Immunoprecipitation and immunoblotting EC after various treatments were washed twice with cold PBS and lysed in 1.5 ml of cold lysis buffer (50 mM Tris-HC, pH 7.6, 150 mM NaCl, 0.1% Triton X-100, 0.75% Brij 96, 10 μg/ml aprotinin, 10 μg/ml leupeptin, 2 mM PMSF, 1 mM EDTA) for 20 min on ice. Protein concentrations were determined with a Bio-Rad kit. For immunoprecipitation to analyze protein interaction in vivo, 400 μg of cell lysate supematant were incubated with 5 μg of the first protein-specific antiserum (e.g. anti-Trx from Medical & Biological Laboratory) for 2 h with 50 μl of GammaBind plus Sepharose. Immune complexes were collected after each immunoprecipitation by centrifugation at 14,000× g for 10 min followed by 4 washes with lysis buffer. The immune complexes were subjected to Western blot with the second protein (e.g., ASK1)-specific antibody (Santa Cruz Biotech, Santa Cruz, Calif.). The chemiluminescence was detected using an ECL kit according to the instructions of the manufacturer (Amersham Life Science, Arlington Heights, Ill.). For Flag-tagged and HA-tagged proteins, anti-Flag M2 antibody (Sigma) and anti-HA antibody (Roche Diagnostics) were used, respectively.

ASK1 and JNK kinase assays ASK1 and JNK assays were performed as described previously (Liu Y et al. Laminar flow inhibits TNF-induced ASK1 activation by preventing dissociation of ASK1 from its inhibitor 14-3-3. J Clin Invest. 107:917-23 (2001)); Min W, Pober J S. TNF initiates E-selectin transcription in human endothelial cells through parallel TRAF-NF-kappa B and TRAF-RAC/CDC42-JNK-c-Jun/ATF2 pathways. J Immunol. 159:3508-18 (1997)) using GST-MKK4 and GST-c-Jun (1-80) fusion protein as a substrate, respectively. Briefly, total 400 μg cell lysates were immunoprecipitated with 5 μg of antibody against ASK1 or JNK1 (Santa Cruz). The immunoprecipitates were mixed with 10 μg GST-MKK4 or GST-c-Jun (1-80) suspended in the kinase buffer (20 mM Hepes, pH 7.6, 20 mM MgCl₂, 25 mM β-glycerophosphate, 100 μM sodium orthovanadate, 2 mM DTT, 20 μM ATP) containing 1 μl (10 μCi) of [γ-32P] ATP. The kinase assay was performed at 25° C. for 30 min. The reaction was terminated by the addition of Laemmli sample buffer and the phosphorylated GST-MKK4 or GST-c-Jun (1-80) was visualized by autoradiography.

GST-Trx pull-down assay: GST fusion protein preparation and GST pull-down assay were performed as described previously. (Liu Y et al. Laminar flow inhibits TNF-induced ASK1 activation by preventing dissociation of ASK1 from its inhibitor 14-3-3. J Clin Invest. 107:917-23 (2001)). Briefly, GST-Trx fusion proteins expressed in Escherichia coli XL-1 blue were affinity purified on glutathione-Sepharose beads (Pharmacia). 400 μg of cell lysates expressing HA-tagged ASK1 were incubated with 10 μg of GST-Trx bound to glutathione-Sepharose in the lysis buffer containing either 1 mM DTT or 1 mM H₂O₂. The beads were washed 4 times with the lysis buffer before the addition of boiling Laemmli sample buffer. Bound ASK1 proteins were resolved on SDS-PAGE and detected by Western blot with anti-HA.

Quantitation of cell killing: Cell killing assay was performed as described previously with a modification (Slowik M R et al. Evidence that tumor necrosis factor triggers apoptosis in human endothelial cells by interleukin-1-converting enzyme-like protease- dependent and -independent pathways. Lab Invest. 77:257-67 (1997)). EC were transfected with a combination of GFP reporter plasmid and the control vector or experimental expression plasmids for ASK1 and Trx at 1:1 ratio as indicated. GFP-positive cells were visualized under a fluorescence microscope and counted as number of survival cells.

Caspase 3 activity assay: Caspase 3 activity was measured with a Caspase 3 fluorescence kit (Sigma) according to the Manufacturer's protocol. Briefly, BAEC were harvested in Caspase 3 lysis buffer (25 mM Hepes, pH 7.4, 5 mM CHAPS, 5 mM DTT) and incubated on ice for 15-20 min followed by a centrifugation at 14,000× g for 10-15 min at 4° C. For each reaction, 5 μl (200 μg) of cell lysate was incubated with 200 μl of 16 μM Caspase 3 peptide substrate acetyl-ASP-Glu-Val-Asp-7 amido-4-methylcoumarin (Ac-DEVD-AMC) in the assay buffer (25 mM Hepes, pH 7.4, 5 mM EDTA, 0.1% CHAPS, 5 mM DTT) in the presence or absence of 100 μM Caspase 3 inhibitor (Ac-DEVD-CHO). The reaction was incubated in the dark for 1-1.5 h and fluorescence was measured in a fluorescence plate reader. The measured fluorescence was used as an arbitrary unit.

Results

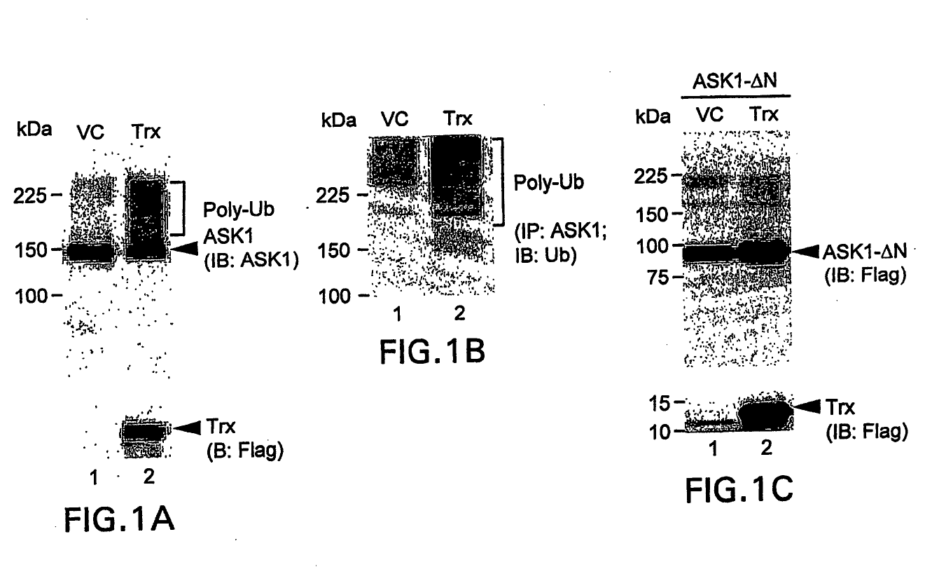

Trx induces ASK1 ubiquitination and degradation in EC. To determine if Trx affects ASK1 protein stability, BAEC were transfected with Trx-expressing plasmid or a control vector (VC) with Lipofactamine2000. Transfection efficiency was determined by transfection of a GFP construct under a fluorescence microscope and usually reached 90% in BAEC and 10% in HUVEC. The high transfection efficiency in BAEC allowed us to examine effects of transgene on endogenous ASK1. Endogenous ASK1 was determined by Western blot with anti-ASK1. Results showed that overexpression of Trx in BAEC decreased protein level of ASK1 with a concomitant increase of high molecular bands above ASK1 protein compared to VC-transfected cells (FIG. 1A, compare lane 2 to lane 1). The high molecular bands are usually ubiquitination of target proteins for degradation (Laney J D et al. Substrate targeting in the ubiquitin system. Cell 97:427-30 (1999)). Indeed, these high molecular mass above ASK1 protein were shown to be poly-ubiquitinated (Ub) ASK1 proteins as demonstrated by immunoprecipitation with anti-ASK1 followed by Western blot with anti-Ub (FIG. 1B). The basal ubiquitination of ASK1 was detected (lane 1 in FIG. 1B) and Trx expression significantly increased ASK1 ubiquitination (lane 2 in FIG. 1B). The ubiquitination of ASK1 was also confirmed by co-expression of ASK1 and HA-tag Ub (Treier et al. Ubiquitin-dependent c-Jun degradation in vivo is mediated by the delta domain. Cell 78:787-98 (1994)). These results indicate that Trx overexpression in EC induced ASK1 ubiqutination and degradation. In contrast, expression of Trx did not induce ubiquitination and degradation of ASK1 -ΔN, a mutant ASK1 lacking the N-terminal Trx-binding domain (FIG. 1C). These data suggest that association of Trx with ASK1 (the fiill-length ASK1) is required for Trx-induced ASK1 ubiquitination and degradation.

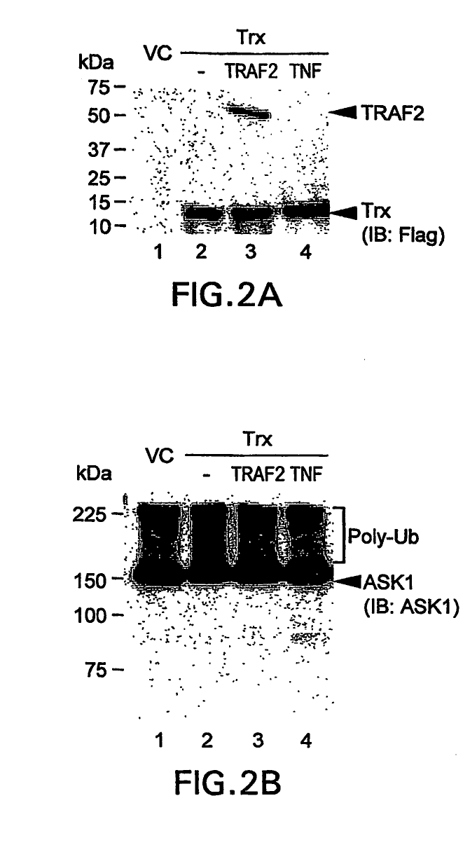

TNF and TRAF2 block Trx-induced ASK1 ubiquitination and degradation. TNF through the adaptor protein TRAF2 activates ASK1, in part, by dissociating ASK1 from Trx (Gotoh Y et al. Reactive oxygen species- and dimerization-induced activation of apoptosis signal-regulating kinase 1 in tumor necrosis factor-alpha signal transduction. J Biol Chem. 273:17477-82 (1998); Liu H et al. Activation of apoptosis signal-regulating kinase 1 (ASK1) by tumor necrosis factor receptor-associated factor 2 requires prior dissociation of the ASK1 inhibitor thioredoxin. Mol Cell Biol. 20:2198-208 (2000)). To examine if TNF and TRAF2 reverse Trx-induced ASK1 ubiquitination/degradation, BAEC were either co-transfected with expression constructs for Trx and TRAF2 (both were Flag-tagged), or transfection with Trx followed by treatment with TNF (10 ng/ml for 15 min). Expression of Trx and TRAF2 was determined by Western blot with anti-Flag (FIG. 2A). Ubiquitination and degradation of endogenous ASK1 in EC were determined as described above. Results showed that TRAF2 expression and TNF treatment blocked Trx-induced ASK1 ubiquitination (increase of high molecular mass) and ASK1 degradation (decrease of 170 kD ASK1 band) compare lanes 3, 4 to lane 2 in FIG. 2B). These data further support that Trx regulates ASK1 ubiquitination and degradation.

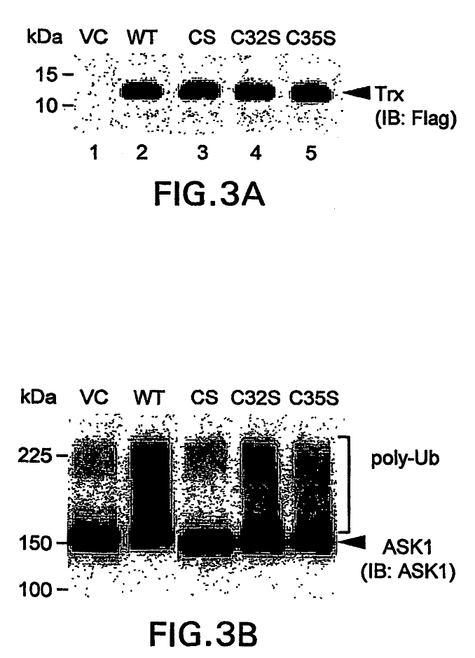

Redox activity of Trx is not requiredfor induction ofASK1 ubiquitination and degradation. To examine the role Trx redox activity in promoting ASK1 ubiquitination and degradation, a single-mutant of Trx at the catalytic site C32 or C35 (Trx-C32S or Trx-C35S) and a double-mutant of C32S and C35S (Trx-CS) were generated. Trx-C32S and Trx-C35S, like Trx-CS, are catalytically inactive (Powis G et al. The role of the redox protein thioredoxin in cell growth and cancer. Free Radic Biol Med. 29:312-22 (2000); Holmgren A. Thioredoxin. Annu Rev Biochem. 54:237-71 (1985); Holmgren A. Thioredoxin and glutaredoxin systems. J Biol Chem. 264:13963-6 (1989)). BAEC were transfected with Flag-tagged Trx expression constructs, and Trx protein was determined by Western blot with anti-Flag. Results showed the equal amount of Trx proteins were expressed (FIG. 3A). Endogenous ASK1 protein in EC was determined by Western blot with anti-ASK1. To our surprise, Trx-C32S and Trx-C35S (but not Trx-CS), like Trx-WT, increased ASK1 poly-ubiquitination with concomitant decrease in ASK1 protein (FIG. 3B). These data suggest that Trx redox activity is not required for its ability to induce ASK1 ubiquitination/degradation.

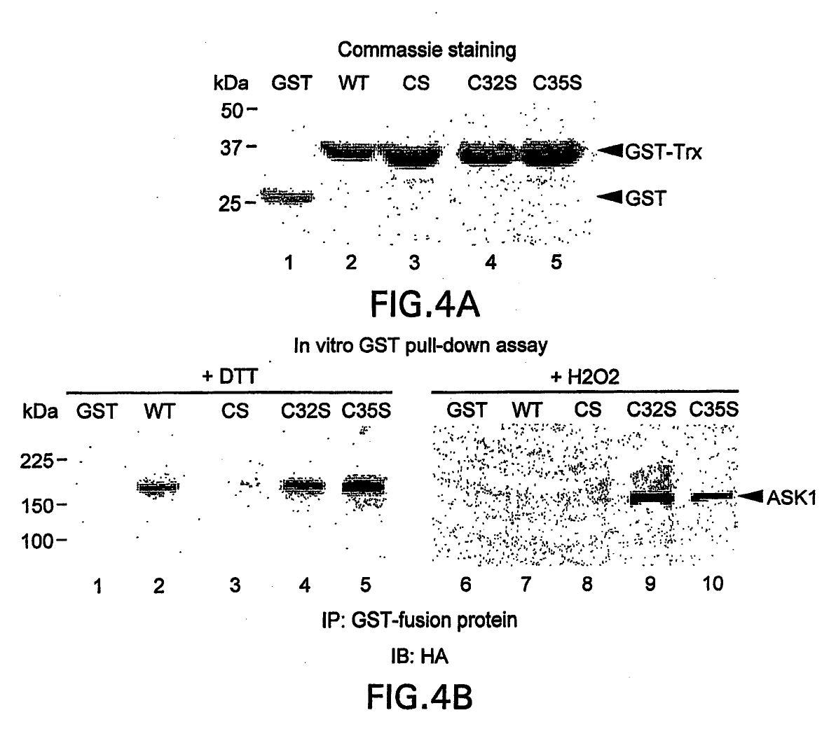

Hydrogen peroxide dissociates wild type Trx but not Trx-C32S or Trx-C35S from ASK1 in vitro. Association of ASK1 with various Trx proteins - Trx-WT, Trx-CS, Trx-C32S or Trx-C35S in the presence of 1 mM DTT or H₂0₂ in an in vitro GST pull-down assay. Bacteria-expressed GST-Trx proteins were purified and protein concentrations were determined by SDS-PAGE (FIG. 2A). Then BAEC lysates containing HA-tag ASK1-WT (Liu Y et al. Laminar flow inhibits TNF-induced ASK1 activation by preventing dissociation of ASK1 from its inhibitor 14-3-3. J Clin Invest 107:917-23 (2001)) were used for GST pull-down assay. ASK1 bound to GST-Trx was determined by Western blot with anti-HA. The results showed that in the presence of 1 mM DTT (Trx remains in a reduced form under this condition), ASK1 bound to Trx-WT, Trx-C32S and Trx-C35S (but not Trx-CS) (lanes 1-5 in FIG. 2B). As expected, addition of 1 mM H₂O₂ disrupted the association of Trx-WT with ASK1 by oxidizing Trx-WT (lane 2 in FIG. 4A vs lanes 7 in FIG. 4B). In contrast, Trx-C32S and Trx-C35S retained their associations with ASK1 in the presence of H₂O₂ (lanes 4-5 vs lanes 9-10 in FIG. 4B). These data suggest that binding of Trx-C32S and Trx-C35 with ASK1 is ROS-resistant, most likely because the mutants cannot form an intramolecular disulfide bond. GST or GST-Trx-CS did not bind to ASK1, indicating that a single Cys residue of Trx (C32 or C35) is necessary and sufficient for ASK1 binding.

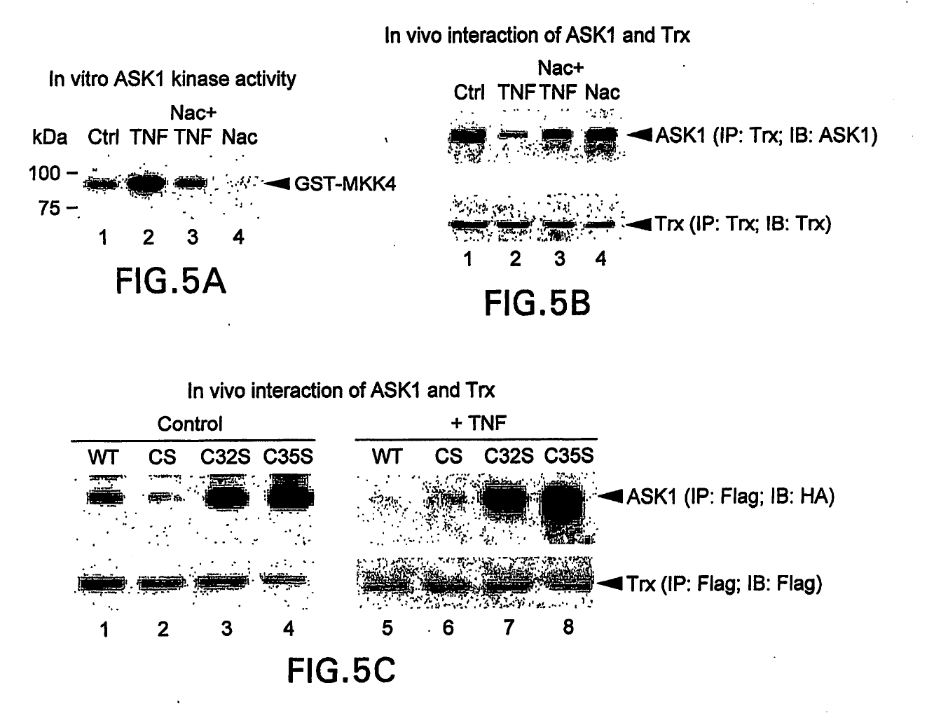

TNF dissociates wild type Trx but not Trx-C32S or Trx-C35Sfroin ASK1 in vivo. Trx-C32S and Trx-C35S were examiner to determine whether they constitutively bind to ASK1 in vivo. Regulation of ASK1 by Trx was first examined to determine if such regulation is ROS-dependent in EC. BAEC were either untreated or treated with N-acetyl-cysteine (Nac, 1 mM) or vehicle for 60 min prior to TNF-α (10 ng/ml) stimulation for 15 min. TNF-induced ASK1 activation was measured by an in vitro kinase assay using GST-MKK4 (JNKK1) fusion protein as a substrate. TNF activated ASK1 in EC (FIG. 5A). Pre-exposing EC to Nac significantly inhibited TNF-stimulated ASK1 activity (70% inhibition, n=3, p<0.01), suggesting that regulation of ASK1 by Trx in EC is ROS-dependent. Association of ASK1 with Trx was easily detected in untreated EC cells (FIG. 5B, Ctrl). TNF treatment significantly decreased the interaction of ASK1 with Trx, indicating that TNF activates ASK1, in part, by dissociating ASK1 from Trx in EC. In contrast, Nac pretreatment prevented TNF-induced dissociation of ASK1 from Trx. Similar results were obtained in human EC (HUVEC). These data suggest that the association of Trx with ASK1 is ROS-dependent, and TNF activates ASK1 in EC, in part, by generating ROS to oxidize Trx leading to dissociation of ASK1 from Trx.

To examine association of Trx mutants with ASK1 in vivo, the Flag-tagged Trx construct (WT, CS, C32S or C35S) was co-transfected with HA-ASK1 into BAEC and the interaction of these Trx proteins with ASK1 were examined by co-immunoprecipitation assay. As expected, Trx-WT, Trx-C32S and Trx-Trx-C35S (but not Trx-CS) bound to ASK1 in resting EC (FIG. 5C). Trx-WT exists in both reduced form and oxidized form (Holmgren A. Thioredoxin. Annu Rev Biochem. 54:237-71 (1985); Holmgren A. Thioredoxin and glutaredoxin systems. J Biol Chem. 264:13963-6 (1989)) and showed a weaker binding for ASK1 than Trx-C32S and Trx-C35S (FIG. 5C). TNF treatment completely dissociated Trx-WT from ASK1 (FIG. 5C, lanes 1 and 5). In contrast, association of Trx-C32S or Trx-C35S with ASK1 was not reduced by TNF treatment, indicating that they remain in a complex with ASK1 (FIG. 5C). These data demonstrate that Trx-C32S and Trx-C35S, unlike Trx-WT, bind to ASK1 in a TNF and ROS-resistant manner.

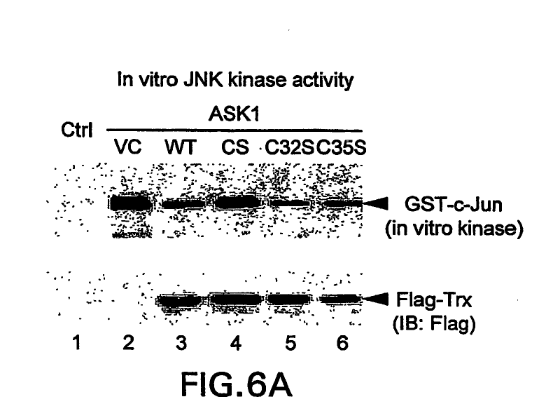

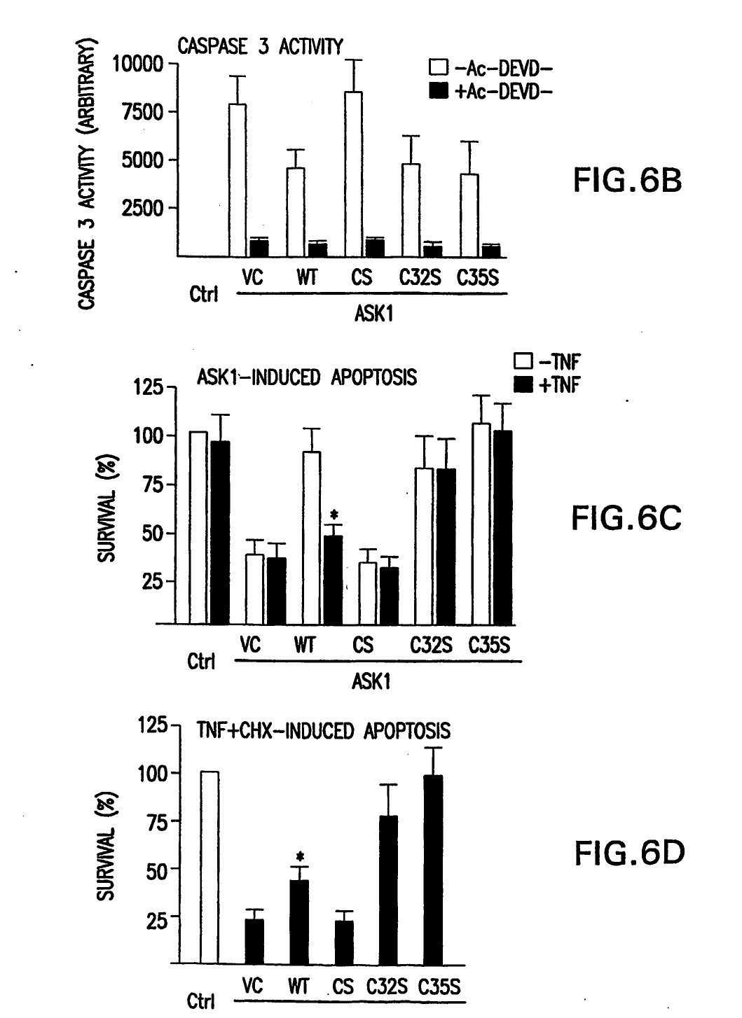

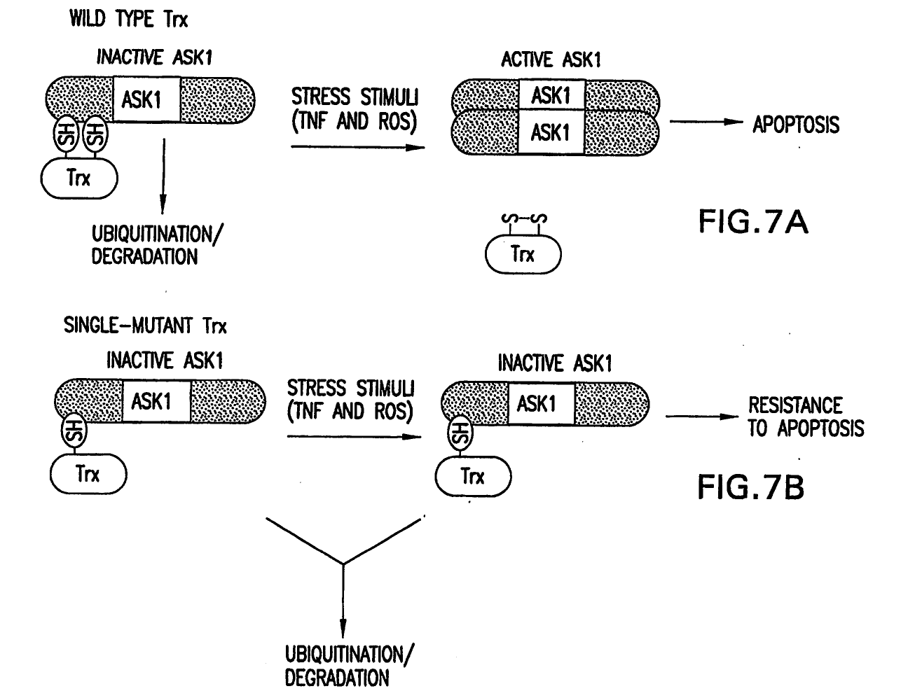

Trx-C32S and Trx-C35S (but not Trx-WT) inhibits ASK1-mediated EC apoptosis induced by TNF. To determine the biological consequence of Trx-induced ASK1 ubiquitination/degradation, the effects of Trx on ASK1-induced apoptosis were examined. ASK1-induced activation of JNK and caspase 3 has been implicated in cell d e a t h (Hatai T et al. Execution of apoptosis signal-regulating kinase 1 (ASK1) induced apoptosis by the mitochondria-dependent caspase activation. J Biol Chem.275:26576-81 (2000); Tournier C et al. Requirement of JNK for stress-induced activation of the cytochrome c- mediated death pathway. Science. 288:870-4 (2000)). First, the effects of Trx on JNK activation induced by ASK1 were examined. BAEC were co-transfected with ASK1 and Trx expression constructs as indicated, and JNK activity was measured by an in vitro kinase assay using GST-c-Jun as a substrate. Results showed that ASK1 expression in EC activated JNK (Compare ASK1NC to Ctrl in FIG. 6A). Co-expression of Trx-WT, Trx-C32S and Trx-C35S (but not Trx-CS) inhibited ASK1-induced JNK activity (FIG. 6A top panel). Western blot with anti-Flag indicated equal amounts of Trx proteins were expressed (FIG. 6A bottom panel). Similar results were obtained in human EC (HUVEC).

Then the effects. of Trx on ASK1-induced caspase 3 activation, a hallmark of the execution of apoptotic cell death (Green D R. Apoptotic pathways: paper wraps stone blunts scissors. Cell. 102:1-4 (2000); Hengartner MO. The biochemistry of apoptosis. Nature. 407:770-6 2000)) was examined. Caspase 3 activity was determined by an in vitro assay using peptide substrate acetyl-ASP-Glu-Val-Asp-7 amido-4-methylcounarin (Ac-DEVD-AMC). Overexpression of ASK1 in BAEC increased caspase 3 activity compared to the control cells (FIG. 6B). Caspase 3 activity was specifically inhibited by the presence of the caspase 3 inhibitor (+Ac-DEVD-CHO). Co-expression of Trx-WT, Trx-C32S or Trx-C35S significantly (n=4 and p<0.05) inhibited ASK1-induced caspase 3 activity (50±2%, 48±4% and 52±5%, respectively). However, Trx-CS failed to block ASK -induced caspase 3 activation (FIG. 6B).

ASK1-induced EC death was measured by a GFP co-transfection killing assay as previously described with minor modifications (Slowik M R Evidence that tumor necrosis factor triggers apoptosis in human endothelial cells by interleukin-1-converting enzyme-like protease- dependent and -independent pathways. Lab Invest. 77:257-67 (1997)). Overexpression of ASK1 in EC (BAEC or HUVEC) induced 60% cell death at 48 h post-transfection, i.e., 40% of GFP-positive (survival) EC compared to the control cells (Ctrl as 100% survival, FIG. 6C). Consistent with binding activities for ASK1, co-expression of Trx-WT, Trx-C32S and Trx-C35S (but not Trx-CS) significantly (n=4 and p<0.05) inhibited ASK1-induced EC death and increased cell survival to 92±5, 80±8, and 105±6%, respectively (FIG. 6C, white bars). In contrast, Trx did not inhibit ASK1-ΔN-induced apoptosis. TNF treatment (to generate ROS and oxidize Trx) specifically diminished the inhibitory effect of Trx-WT on ASK1-induced apoptosis (FIG. 6C, stripped bars). In contrast, the single-mutations (Trx-C32S and Trx-C35S) did not respond to TNF treatment and retained their inhibitory effects on ASK-1-induced apoptosis (FIG. 6C, stripped bars).

Finally, the effects of Trx on TNF-induced cell death was examined. TNF alone does not induce EC apoptosis. However, TNF in the presence of protein synthesis inhibitor cycloheximide (CHX) strongly induces ASK1 activation (Liu Y, et al. Laminar flow inhibits TNF-induced ASK1 activation by preventing dissociation of ASK1 from its inhibitor 14-3-3. J Clin Invest. 107:917-23 (2001)) and EC apoptosis (Slowik M R et al. Evidence that tumor necrosis factor triggers apoptosis in human endothelial cells by interleukin-l-converting enzyme-like protease- dependent and —independent pathways. Lab Invest. 77:257-67. (1997)) EC were co-transfected with GFP and Trx as indicated followed by TNF+CHX treatment. Effects of Trx expression on TNF+CHX-induced EC death were measured by counting GFP-positive cells. TNF+CHX induced in BAEC induced 75% EC death at 24 h post-treatment, i.e., 25% of GFP-positive (survival) EC compared to the control cells (Ctrl as 100% survival, FIG. 6D). Consistent with constitutively binding activity for ASK1, Trx-C32S and Trx-C35S (but not Trx-WT) retained the inhibitory effects on TNF+CHX-induced apoptosis (FIG. 6D). Trx-C35S showed a slightly stronger inhibitory effect on ASK1 activity (100±12% survival) than Trx-C32S (80±10% survival). Similar results were obtained in human EC (HUVEC). These data indicate that Trx-C32S and Trx-C35S inhibit ASK1-mediated EC apoptosis in a TNF-resistant manner.

### EXAMPLE 2

The Effect of Trx on ASK1 Induction of Insulin Resistance

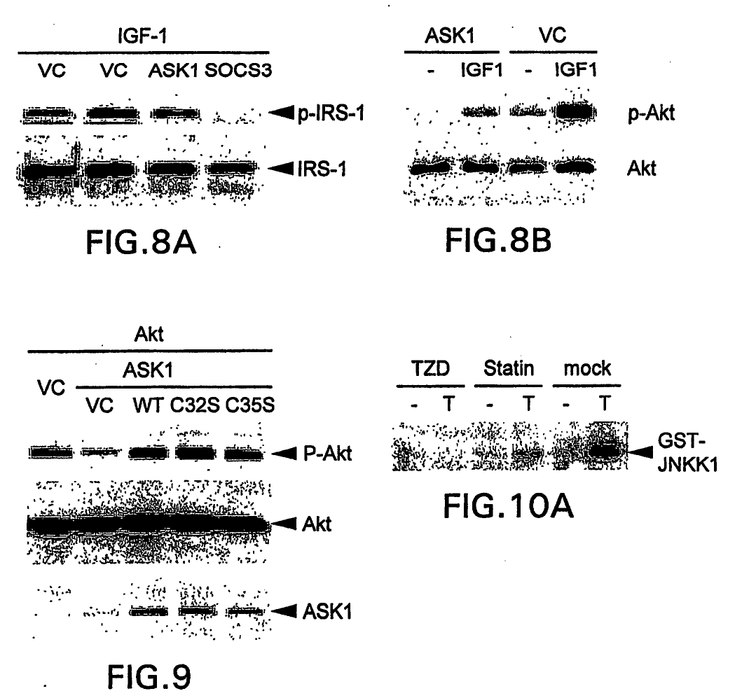

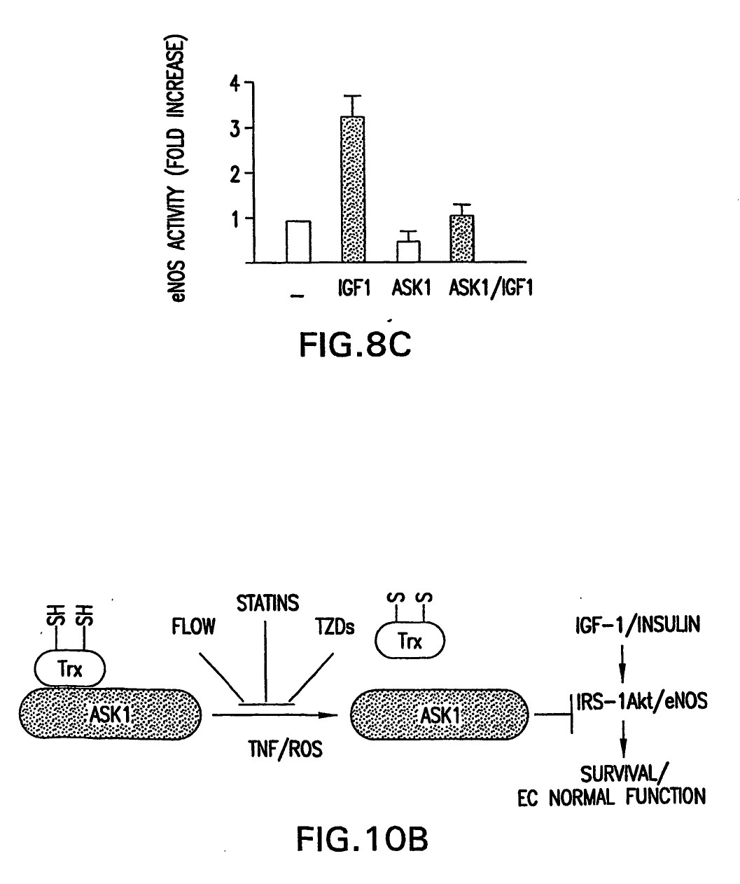

To determine a role of ASK1 in ROS/TNF-induced IR (impairment of insulin/IGF1 signaling) in EC, the effects of ASK1 activation on IGF1-induced phosphorylation of IRS-1, Akt and eNOS activity were examined. IGF-1 was chosen because cultured EC express IGF1 receptor at higher level than that of insulin receptor. BAEC were transfected with VC or ASK1 expression construct, cells were treated with IGF1 (10 ng/ml) for indicated time. SOCS3 was used as a control which has been shown as a suppressor of IRS-1 activation. Activation (tyrosine phosphorylation) of IRS-1 was determined by IP/Western blot with anti-phosphotyrosine. Akt phosphorylation at Ser-473, a hallmarker for Atk activity was determined by Western blot with p-Ser473-specific antibody. The eNOS enzymatic activity was also by an enzymatic assay based on the conversion of 3H-L-arginine to 3H-citrulline (Calbiochem, Nitric Oxide Synthase Assay Kit). Results show that ASK1 expression inhibits IGF1-induced phosphorylation of IRS-1 and Akt and eNOS activity (FIG. 8A, B, C).

To determine if Trx protects ASK1-induced inhibition on IGF1 signaling, Trx and ASK1 were coexpressed as in the above experiments. Results showed that co-expression of Trx-WT, C32S and C35S (but not Trx-CS) prevents ASK1-induced reduction of Akt phosphorylation (FIG. 9). These data indicate that ASK1 is a critical mediator in ROS/TNF-induced IR and that ASK1 may be target for anti-atherosclerotic and anti-diabetic drugs. To test this, the effects of TZDs (insulin-sensitizers) and statins on ASK1 activity were tested. Results showed that TZDs (rosiglitazone) and statins (simvastatin) inhibit TNF-induced ASK1 activation (FIG. 10). Inhibition of ASK1 may due to their anti-oxidant activities of these drugs which may be indirect by inhibiting TNF-dependent generation of ROS.

### EXAMPLE 3

ASK1 Stabilization of TNF-Induced SOCS3 in EC by Phosphorylating SOCS3 at the SOCS-Box

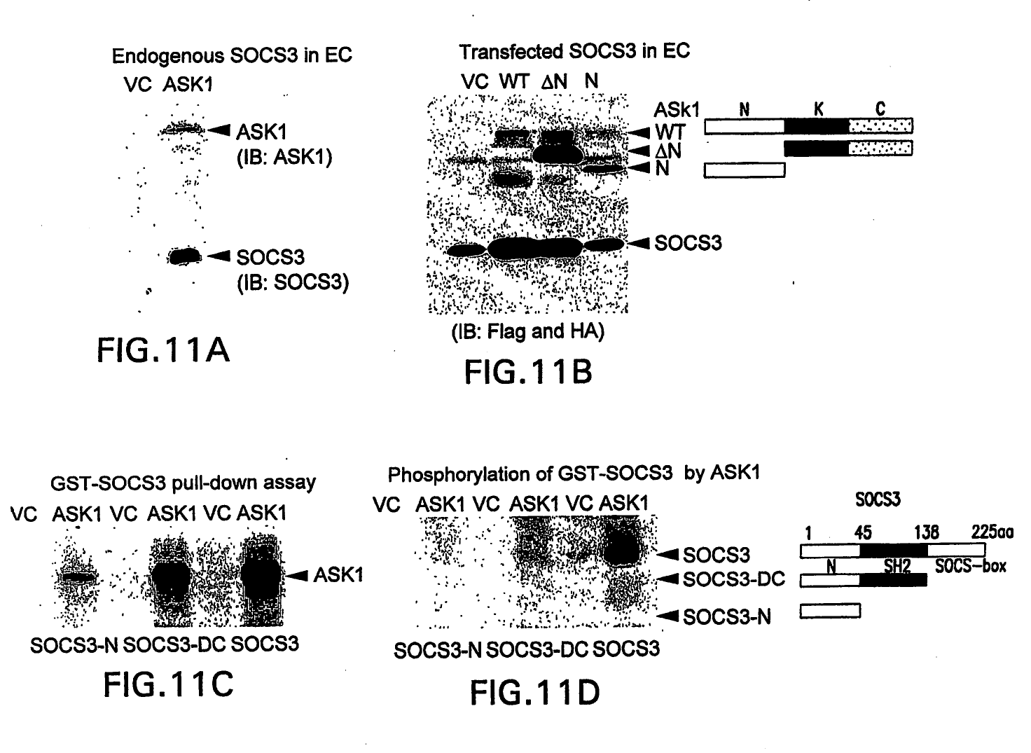

To determine the mechanism for ASK1-induced inhibition of IRS-1/Akt/eNOS activities, ASK1 targets upstream of IRS-1, for example, SOCS3 were tested. First, the effect of TNF on SOCS3 induction/stabilization in EC was examined. TNF treatment for 24 h induced SOCS3 expression, as shown by Western blot. To determine if ASK1 mediated this effect, EC were transfected with VC or ASK1 expression construct and endogenous SOCS3 was detected by Western blot with anti-SOCS3. Results showed that ASK1 expression increased SOCS3 levels dramatically (FIG. 11). To determine if kinase activity of ASK1 was required for SOCS3 stabilization, a SOCS3 (Flag) expression plasmid was co-transfected with ASK1-WT (HA), ASK1-DN (Flag) and ASK1-N (Flag). SOCS3 and ASK1 protein were detected by Western blot with anti-Flag and anti-HA. The results showed that ASK1-WT and ASK1-DN (active), but not VC or ASK1-N (inactive), stabilized SOCS3, suggesting that kinase activity of ASK1 is required for SOCS3 stabilization (FIG. 11B). Thus, ASK1 appears to bind directly to SOCS3 and to phosphorylate SOCS3. Interaction of SOCS3 with ASK1 was confirmed by IP/Westem blot.

To determine the binding and phosphorylation site of SOCS3 by ASK1, GST pull-down assay and in vitro kinase assay using GST-SOCS3 as a substrate were performed. In the pull-down assay using ASK1-expressing lysates, ASK1 associated with the full-length SOCS3, SOCS3-DC(1-138aa, deletion of the C-terminal SOCS box) but not with the SOCS3-N(1-45aa). The full-length SOCS3 bound more ASK1 than SOCS3-DC with equal amount of GST-fusion protein, suggesting that the C-terminal SOCS box and the SH2 domain were required for ASK1 interaction (FIG. 11C). In an in vitro kinase assay, only the full-length GST-SOCS3 was phosphorylated by ASK1 (FIG. 11D), indicating that the phosphorylation site was located within the SOCS-box.

### EXAMPLE 4

EC-Specific Expression of Trx-Transgenic Mice

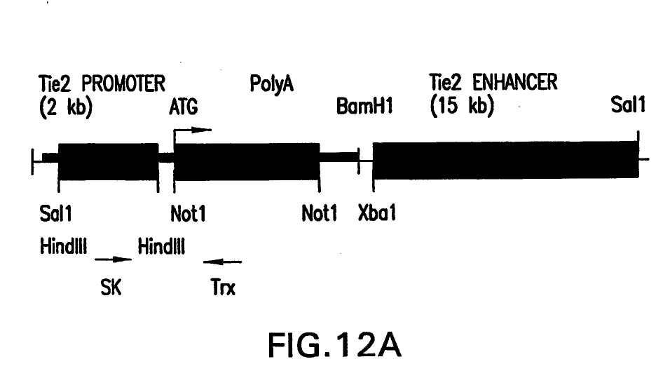

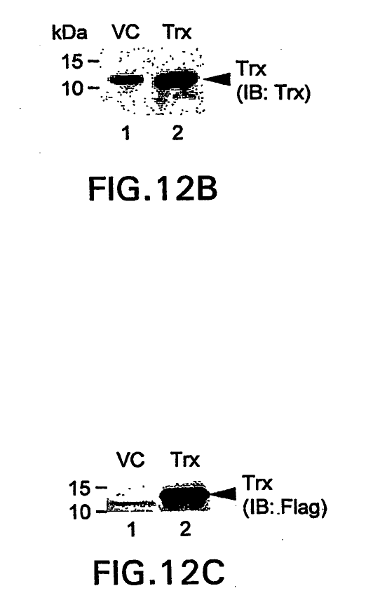

The Tie-2 promoter/enhancer constructs were provided by Dr. Tom Sato (FIG. 12). The longer Tie2 enhancer fragment (15 kb), rather than short one (1.6 kb), was chosen because the longer one obtains uniform vascular expression, especially in adult. The construct was generated in which the Flag-tagged human Trx-C35S was inserted into the transgenic vector between the 2.0-kb murine-Tie2 promoter, 250-bp SV-40 polyadenylation sequences and 15-kb Tie2 full enhancer (FIG. 12A). Expression of Flag-Trx-C35S was determined by Western blot with anti-Trx or anti-Flag (FIG. 12B,C) after transient transfection into BAEC. Tie2-Trx-C35S was not expressed in 293T (non-EC), suggesting that Tie2 promoter drives EC-specific expression of Trx-C35.

### EXAMPLE 5

Effects of Trx Expression on Atherosclerosis Progression in LDLR−/− Mice

Male C57BL/6 and LDLR−/− mice backcrossed 10 generations into the C57BL/6 strain are purchased from Jackson Laboratory (Bar Harbor, ME). The wild type, LDLR−/−, TgTrx-WT/LLDR−/− and TgTrx-C35S/LDLR−/− are carried out at 2 to 4 months of age using 1) untreated standard chow-fed, 2) mice fed for 2 weeks an AIN-76A semipurified cholate-free diet containing high fat (40% of energy intake) and 1.25% choleterol (Research Diets, New Brunswick, N.J., diet no. D12108). Aorta are harvested after perfusion with PBS and used for vascuprotein assay, en face microscopy and histology analyses (after fixation with 2% paraformaldehyde ). Ten mice in each strain are studied. The founder line with high expression of Trx are studies.

Animals are measured by the following assays (Table 1) and all data are compared the differences among the groups by ANOVA analysis (p<0.05).

TABLE 1
Animal analyses

| Assay | Tissue | Method |
|---|---|---|
| Cholesterol level (mg/dl) | Serum from right | calorimetric kit |
| | ventricle | (Roche) |
| Trx/ASK1/SOCS3 expression | Entire aorta | en face confocal |
| Lesion area (plaque/total | Entire aorta | Sudan IV-stained |
| aorta area) | | lesions |
| Plaque cell density | Aortic sinus plaque | nuclei in 3 |
| (cells/mm²) | | midlesion levels |
| SMC content (SMC/cells) | Descending aorta | histology for |
| | | α-actin |

ASK1 and SOCS3 expression and EC apoptosis: The LDLR−/− mice fed a normal chow diet have only a modest elevation in plasma cholesterol and do not develop lesions; however, when fed a chelesterol rich diet, these mice develop complex lesions of atherosclerosis similar to those in human. To determine the pattern of endothelial ASK1 activation and SOCS3 and expression in the LDLR−/− model of atherosclerosis, mice were placed on the cholesterol-rich diet for 0, 4, 8. or 20 weeks. These time points were selected based on preliminary studies that demonstrated lesions began to develop in the aortic arch and near the ostia after about 4 weeks on the diet. En face oil red 0 staining is used to map regions of the LDLR−/− mouse ascending aorta and arch that are highly predisposed or protected from atherosclerotic lesion formation, and those are designated these as high and low probability (HP and LP) regions. The difference in expression of ASK1/SOCS3 is thus assessed in these areas. Plaque area, plaque cell density and intimal smooth muscle cell content are determined.

Plasmid construction. Mammalian expression plasmids for wild-type and the kinase inactive ASK1 were provided by Dr. Genhong Chen (Univ. of California, Los Angeles, CA); for GST-JNKK1 (MKK4) by Dr. Bing Su (M.D. Anderson, TX). Mutations of C32S and C35S in Trx were introduced by recombinant PCR according to the method of Liu, Y. et al. (2001) Flow inhibits TNF-induced ASK1 activation by enhancing interactions of ASK1 with its inhibitor 14-3-3. J Clin. Invest. 107, 917-923, which is incorporated herein by reference in its entirety for the method, and confirmed by DNA sequencing. Expression plasmids for Flag-tagged Trx proteins, and ASK1-N proteins were constructed into the Flag-vector according to the method of Liu, Y. et al. (2001) Flow inhibits TNF-induced ASK1 activation by enhancing interactions of ASK1 with its inhibitor 14-3-3. J. Clin. Invest. 107, 917-923, which is incorporated herein by reference in its entirety for the method.

Cells and cytokines. HUVEC and BAEC are isolated and cultured as described above. Human rTNFα is from R&D Systems, Inc. and used at 100 U/ml and 250 U/ml, respectively.

Transfection and viral infection. For transfection, BAEC is performed by lipofactamine according to manufacturer's protocol (Gibco, Gaithersburg, Md.). Transient transfections of HUVEC are performed using a DEAE-Dextran protocol as described previously by Mn, W. and Pober, J. S. (1997) TNF initiates E-selectin transcription in human endothelial cells through parallel TRAF-NF-κB and TRAF-RAC/CDC42-JNK-ATF2/c-Jun pathways. J. Immunol. 159, 3508-18, which is incorporated herein by reference in its entirety for the method.

Lentiviral Vector Construction, Production, and Assay. A dual cDNA expression cassette vector co-expresses Trx or ASK1 with enhanced green fluorescent protein (EGFP), on a bicistronic MRNA, linked by an internal ribosome entry sequence (IRES) in the pHR′-CMV (a gift from Dr. I. Verma). The viral vector is prepared as previously described in Naldini, L., Blomer, U., Gage, F. H., Trono, D., and Verma, I. M. (1996). Efficient transfer, integration, and sustained long-term expression of the transgene in adult rat brains injected with a lentiviral vector. Proc. Natl. Acad. Sci. USA. 93: 11382-11388, which is incorporated herein in its entirety for the method. Briefly, 1.5×10⁶ 293 cells are plated in 10 cm plates, and transfected the following day with 15μg of pCMVAR8.2, 20 μg of the pHR′ plasmid, and 5 μg of pHCVM-VSVG, by calcium phosphate DNA precipitation. Conditioned medium is harvested 62 hrs after transfection, cleared of debris by low speed centrifugation, filtered through 0.45 mm filters (Falcon™, BD Biociences, Bedford, Mass.). Titering is performed by infecting 293T cells overnight with serial dilutions of vector stock in culture medium supplemented with 8 mg/ml polybrene (Sigma, St. Louis, Mo.). After medium replacement, the cells are further incubated for 36 hr, and expression GFP by FACS analysis. Concentrated vector stocks are prepared by ultracentrifugation of conditioned medium at 50,000× g for 90 min. The final pellet is resuspended in 0.05% of the starting volume in sterile PBS containing 4 mg/ml of polybrene. Stocks are titered as described above and stored frozen at about −80° C. Lentiviral infection is performed at MOI of 5.

JNK and ASK1 kinase assays. JNK assay is performed as described above using GST-c-Jun (1-80) fusion protein as a substrate. ASK1 assay is performed as described above using GST-JNKK1 (MKK4) as a substrate.

Quantitation of cell killing. Cell killing assay is performed as described above. Cells are transfected with a combination of GFP reporter plasmid and the control vector or experimental expression plasmids for ASK1 and Trx as indicated. GFP-positive cells are visualized under a fluorescence microscope and counted as number of survival cells.

Immunoprecipitation and immunoblottting. HUVEC or BAEC cells with various treatments are washed twice with cold PBS and lysed in 1.5 ml of cold lysis buffer (50 mM Tris-HCl, pH 7.6, 150 mM NaCl, 0.1% Triton X-100, 0.75% Brij 96, 1 mM sodium orthovanadate, 1 mM sodium fluoride, 1 mM sodium pyrophosphate, 10 μg/ml aprotinin, 10 μg/ml leupeptin, 2 mM PMSF, 1 mM EDTA) for 20 min on ice. Se methods described above for EC. For immunoprecipitation to analyze protein interaction in vivo, supernatant of cell lysates are precleared by incubating with normal rabbit serum plus GammaBind plus Sepharose beads on rotator at 4° C. overnight. The lysates are then incubated sequentially with the first protein (e.g. ASK1)-specific antiserum (Santa Cruz Biotech, Santa Cruz, Calif.) for 2 h with 50 μl of GammaBind plus Sepharose. Immune complexes are collected after each immunoprecipitation by centrifugation at 13,000× g for 10 min followed by 3 -5 washes with lysis buffer. The immune complexes are subjected to SDS-PAGE followed by immunoblot (Immobilon P, Millipore, Bedford, Mass.) with indicated antibodies. The chemiluminescence is detected using an ECL kit according to the instructions of the manufacturer (Amersham).

Caspase-3 activity assay. Caspase-3 activity is measured with the Caspase-3 Fluorometric kit (Sigma, St Louis, Mo.) according to the manufacturer's protocol.

GST binding assay. GST-Trx proteins is prepared for in vitro binding assay as described previously in Liu, Y. et al. (2001) Laminar flow inhibits TNF-induced ASK1 activation by preventing dissociation of ASK1 from its inhibitor 14-3-3. J Clin Invest. 107: 917-923, which is incorporated herein by reference in its entirety for the assay method.

IP/kinase assay. The immunoprecipitates (with anti-FLAG) are washed twice with kinase buffer and resuspended in 50 μl of kinase buffer. See Min, W., and Pober, J. S. (1997) TNF initiates E-selectin transcription in human endothelial cells through parallel TRAF-NF-kappa B and TRAF-RAC/CDC42-JNK-c-Jun/ATF2 pathways. J Immunol. 159: 3508-3518, which is incorporated herein by reference in its entirety for the treatment of immunopreceitates. Reaction is performed by adding ³²P-\tilde{γ}ATP (50 μCi) at room temperature for 30 min. Reaction is stopped by SDS-sample buffer and subjected to SDS-PAGE and exposure to film.

eNOS activity assay. The conversion of 3H-L-arginine to 3H-L-itrulline is used to determine eNOS activity in EC lysates as described in Garcia-Cardena, G., et aL (1998) Dynamic activation of endothelial nitric oxide synthase by Hsp90. Nature. 392: 821-823, which is incorporated herein in its entirety for the eNOS activity assay.

Genotyping by PCR. Briefly, tail samples are digested in lysis buffer (75 mM NaCl, 25 mM EDTA, 10 mM Tris [pH 8.0], 1% SDS) and 0.4 mg/ml proteinase K. Genomic DNA is precipitated with isopropanol. PCR is performed as follows: 95° C. for 30 seconds, 60° C. for 30 seconds, and 72° C. for 1 minute for 35 cycles, with a final extension step at 72° C. for 10 minutes. Internal control primers are used.

LPS injection and serum TNF-α levels. Using a 26-gauge needle, C57b1/6 mice or Trx transgenic mice are injected intraperitoneally with LPS (Salnonella typhimurium, Difco Laboratories, Mich.) resuspended in sterile water. For TNF-α injections, mice are injected intravenously with a 28-gauge needle via a retro-orbital approach. Sham-injected animals receive diluent. For studies measuring survival, animals are monitored for up to 2 wk. Survival as the end point in these experiments is calculated from the time of treatment using the product limit Kaplan-Meier method. Calculations of the dose leading to 50% lethality (LD50) at a given time after LPS treatment is performed. Serum TNF-α levels are measured by ELISA according to the manufacturer's instructions (Biosource International, Camarillo, Calif.).

Vascular reactivity. Changes in vascular reactivity (e.g., agonist-induced contraction and agonist-induced relaxation) are measured to elucidate the role of Trx in endothelial dysfunction. To assess agonist-induced contraction, contraction of mouse aortic strips is performed as described previously. See Poppa, V. et al. (1998) Endothelial NO synthase is increased in regenerating endothelium after denuding injury of the rat aorta. Arterioscler Thromb Vasc Biol 18, 1312-21, which is incorporated herein in its entirety for the method of assaying vascular reactivity. In brief, thoracic aortas are isolated from mice after anesthesia with intra-peritoneal pentobarbital (60-90 mg/kg) and cleaned of excessive adventitia tissue. Aortic strips are attached to stainless steel holders and suspended in organ chambers (10 ml) with Krebs buffer at 37° C. for 60 min with a change of buffer after each 15 min and aerated constantly with 95% O₂ and 5% CO₂. Aortas are then incubated at 37° C. for 15 min with Ang II, phenylephrine, or KC1. The contractile dose response to Ang II (0.01-1 μM), phenylephrine (0.1-10 μM), and KC1 (10-30 mM) is measured and compared between transgenic and non-transgenic vessels.

To assess agonist-induced relaxation, aortic strips are stretched with a preload of 1 gm, allowed to equilibrate for 75 min, and then precontracted with 1 μM phenylephrine. The force of contraction is similar for all tissues. Strips are then exposed to increasing concentrations of either ACh (1 nM-10 μM) and NTG (1 nM-30 μM). After the addition of each concentration of drug, the subsequent dose is not added until the baseline is again stabilized. Data are calculated as percentage relaxation relative to phenylephrine-induced contraction. ANOVA and Tukey's post-hoc analysis for inter-group comparisons are used for all statistical analyses.

EC apoptosis assay in vivo (TUNEL/CD31). TUNEL and CD3 1 staining are performed according to methods known in the art. See Haimovitz-Friedman, A., et al., (1997) Lipopolysaccharide induces disseminated endothelial apoptosis requiring ceramide generation. J Exp Med. 186: 1831-1841, which is incorporated herein in its entirety for the assay method.

Plaque immunohistochemistry. Plaque immunohisytochemistry is performed according to methods know in the art. See Moulton, K. S. et al. (1999) Angiogenesis inhibitors endostatin or TNP-470 reduce intimal neovascularization and plaque growth in apolipoprotein E-deficient mice Circulation. 99: 1726-1732, which is incorporated herein in its entirety for the assay method.

Statistical Analysis. Data are presented as means (+SEM). Analysis of densitometry is performed using NIH Image 1.60. Protein phosphorylation is normalized to the amount of immunoprecipitated protein based on Western blot density. Results are then normalized for comparison among different experiments by arbitrarily setting the densitometric value of control cells to 1.0. Significant differences are determined by Student's test (p<0.05). For in vivo studies, experiments are repeated twice and significant differences are analyzed by ANOVA (p<0.05).

### EXAMPLE 6

ASK1 Associates with Troponin T and Induces Troponin T Phosphorylation and Contractile Dysfunction in Cardiomyocytes

Materials and Methods

Plasmid construction: ASK1 -ΔN was amplified by PCR using a 5′ primer with NdeI site and a 3′ primer with SalI. The PCR product was inserted into NdeI and SalI sites of the expression vector pAS2.1 (Clontech) to generate pAS-ASK1-ΔN in which ASK1-ΔN was fused in-frame with the DNA binding domain of yeast transcriptional activator GAL4. The fall-length human cTnT cDNA (accession No NM_{—}000364) was amplified by PCR from a human heart cDNA library (Clontech) using a 5′ primer with EcoRI site and a 3′ primer with XhoI site. The PCR was cloned into mammalian expression Flag-vector (Flag-cTnT) or bacterial expression pGEX-kg vector (GST-cTnT). Similar expression constructs for human cTnI (accession No NM 000363) were also constructed. The mutant cTnT (T194A and S198A) was constructed by site-directed mutagenesis using Quickchange™ site-directed mutagenesis kit (Stratagene) according to the protocol of the manufacturer. A pair of primers was used to introduce,these desired mutations. The sense primer was: 5′-GAA GCA GGC CCA GGC AGA GCG GAA AGC TGG GAA GAG GCA G-3′(SEQ ID NO:20) (the G is mutated from A and the GC is mutated from AG).

Yeast two-hybrid screening: ASK1-ΔN bait was used to screen a pretransformed human heart cDNA library (Clontech). The yeast two-hybrid screening was performed according to the instructions of the manufacturer (Clontech). In brief, the yeast strain AH109 harboring pAS-ASK1-ΔN was mated with Y190 harboring a human heart cDNA library. Mating zygotes were selected on synthetic dropout agar plates lacling Trp, Leu, His and Ade (QDO). Yeast colonies were transferred onto a nylon membrane and processed by the β-galactosidase filter assay. Plasmids from positive colonies were isolated and re-transformed into the yeast strain Y190 with either pAS2.1 or pAS-ASK1-ΔN to confirm that growth on QDO and β-gal was ASK1-ΔN-dependent. The cDNA inserts from true positive clones were subjected to DNA sequencing with a dye terminator cycle sequencing kit (UR core facility).

Preparation of contractile neonatal and adult rat cardiomyocytes: The method for myocyte isolation from adult rats has been described previously (Sheu SS Measurement of cytosolic free calcium concentration in isolated rat ventricular myocytes with quin 2. Circ Res. 55:8304 (1984)). Briefly, isolated myocytes were prepared using a Langendorff setup with retrograde perfusion of collagenase solution (Worthington, type II). This enzyme solution was recirculated through the heart for approximately 30 minutes. The isolated myocytes were kept in standard solution that contained (in mM): 140 NaCl, 5 KC1, 2 CaCi₂, 2 MgCl₂, 10 N-2-hydroxyethyl-piperazine-N-2-ethanesulfonic acid (HEPES), 11 glucose, and pH 7.4 at 37° C. with NaOH. Isolated cells were used for experiments on the same day.

Primary cultures of neonatal rat cardiac ventricular myocytes were prepared by the method of Simpson and Savion (Simpson P et al. Differentiation of rat myocytes in single cell cultures with and without proliferating nonmyocardial cells. Cross-striations, ultrastructure, and chronotropic response to isoproterenol. Circ Res. 50:101-16 (1982)) with minor modifications. Briefly, the ventricles were excised from 1-2 day old Wistar rats under ethyl ether anesthesia and minced with scissors in Ca²⁺ free Krebs-Henseleit buffer (KHB) solution. The cells were dispersed by adding KHB containing 0.05% trypsin and 0.05% collagenase type II. They were stirred with the use of a small magnetic stirrer bar at 37° C. for 10 min, and the supernatants were saved in cold DMEM containing 10% fetal calf serum (complete DMEM), and digestion was repeated four times. The resultant cell suspension was centrifuged, and the pellet was resuspended in the medium. The cells were plated onto culture flasks for 1.5 hours to remove non-cardiac myocytes. The unattached cells were removed and seeded at 4×10⁵ per well in 2.2 cm multi-well gelatin-coated culture plates in DMEM containing 10% fetal calf serum, 100 U/ml penicillin and 100 μg/ml streptomycin. After incubation at 37° C., 5% CO₂ for 48 h, the medium was replaced by complete DMEM containing 101 g/ml sodium transferin, 6.7 ng/ml sodium selenite, 2.01 g/ml ethanolamine, 0.1% BSA and the above antibiotics, and the cells were incubated for further experiments.

GSTpull-down assay: GST-cTnT proteins expression, purification and GST pull-down assay were performed as described previously (Liu Y et al. Laminar flow inhibits TNF-induced ASK1 activation by preventing dissociation of ASK1 from its inhibitor 14-3-3. J Clin Invest. 107:917-23 (2001); Liu Y et al. Thioredoxin promotes ASK1 ubiquitination and degradation to inhibit ASK1-mediated apoptosis in a redox activity-independent manner. Cir. Res. 90:In press (2002)).

cTn Tphosphorylation in vitro and in vivo: cTnT phosphorylation in vitro by ASK1 was performed as follows: ASK1 -ΔN expression plasmid was transfected into 293T cells and ASK1-ΔN protein was immunoprecipitated with anti-Flag. 10 μg of native cTn protein complex (cTnT/I/C) purified from human heart tissue (Research Diagnosis) or GST-cTnT (purified in the lab) was mixed with the ASK1-ΔN immunoprecipitate in a kinase buffer containing 0.2 mM [γ-³²P]ATP . The sample was incubated at 30° C. for 30 min followed by SDS-PAGE and autoradiography. cTnT phosphorylation in vivo by H₂O₂ in cardiomyocytes or by overexpressed ASK1-ΔN in 293T cells was examined. For cardiomyocytes, cells were treated with 100 μM of H₂O₂ for 0-15 min as indicated and phosphorylation of endogenous cTnT was determined. For 293T, cells were transfected with Flag-cTnT and Flag-ASK1-ΔN and phosphorylation of Flag-cTnT was determined at 24 h post-transfection. cTnT phosphorylation was performed as following: culture medium was removed and cells were washed three times with phosphate-free Krebs-Henseleit buffer (KHB) solution (118 mM NaCl, 4.0 mM KC1, 1.2 MM MgCl₂, 1.1 mM K₂HPO₄, 25 mM NaHCO₃, 5.0 mM glucose, 20 mM HEPES, pH7.4) supplemented with 1.8 mM CaCl₂ and 0.1% BSA. Each culture plate was then added with 0.3 ml of the same medium containing carrier-free [³²p] orthophosphate (0.3mCi/ml) and incubated at 37° C. for 2.5 h. Medium was removed and the cells were washed with ice-cold wash buffer (150 mM NaCl, 20 mM HEPES, pH 7.4), solubilized in 100 μl 0.1% SDS, 6.6% glycerol, 3.3% 2-mercaptoethanol, 65 mM Tris-HCl, pH 6.8, and frozen at −20° C. cTnT phosphorylation was analyzed by immunoprecipitation with anti-Flag or anti-cTnT followed by SDS-PAGE and autoradiography.

ASK1 kinase assays: Cardiomyocytes were treated with H₂O₂ (100 μM) for 0-15 min. Cells were lysed in cold lysis buffer and ASK1 activity was measured by an in vitro kinase assay using GST-MKK4 as a substrate as described (Simpson P et al. Differentiation of rat myocytes in single cell cultures with and without proliferating nonmyocardial cells. Cross-striations, ultrastructure, and chronotropic response to isoproterenol. Circ Res. 50:101-16 (1982)).

Lentiviral vector construction, production and assay: A dual cDNA expression cassette vector was constructed to co-express Flag-tagged ASK1-ΔN with GFP, on a bi-cistronic mRNA, linked by IRES in the pHR′-CMV (a gift from Dr. I. Verma). The viral vector was prepared as previously described (Naldini L et al. In vivo gene delivery and stable transduction of nondividing cells by a lentiviral vector. Science 272:263-7 (1996); Yin G et al. Endostatin gene transfer inhibits joint angiogenesis and pannus formation in inflammatory arthritis. Molecular Therapy 5:547-554 (2002)). The final pellet was resuspended in 0.05% of the starting volume in sterile PBS containing 4 μg/ml of polybrene. Stocks were titred as described above and stored frozen at −0° C.

Transfection and viral infection: Transfection of 293T was performed by Lipofectamine according to Manufacturer's protocol (Gibco). Lentiviral infection of cardiomyocytes was performed at MOI of 50 for 24 h as described (Yin G et al. Endostatin gene transfer inhibits joint angiogenesis and pannus formation in inflammatory arthritis. Molecular Therapy 5:547-554 (2002)).

Immunoprecipitation and immunoblotting Cells were washed twice with cold PBS and lysed in 1.5 ml of cold lysis buffer (50 mM Tris-HCl, pH 7.6, 150 MM NaCl, 0.1% Triton X-100, 0.75% Brij 96, 1 mM sodium orthovanadate, 1 mM sodium fluoride, 1 mM sodium pyrophosphate, 10 μg/ml aprotinin, 10 μg/ml leupeptin, 2 mM PMSF, 1 mM EDTA) for 20 min on ice. For immunoprecipitation to analyze protein interaction in vivo, supernatants of cell lysates were precleared by incubating with normal rabbit serum plus GammaBind plus Sepharose beads on rotator at 4° C. overnight. The lysates were then incubated with the first protein-specific antiserum (e.g. cTnT, Santa Cruz Biotech, Santa Cruz, Calif.) for 2 h with 50 μl of GammaBind plus Sepharose. Immune complexes were collected after each immunoprecipitation by centrifugation at 13,000 g for 10 min followed by 3-5 washes with lysis buffer. The immune complexes were subjected to SDS-PAGE followed by immunoblot (Imnobilon P, Millipore, Milford, Mass.) with the second protein (e.g., ASK1)-specific antibody (H300, Santa Cruz Biotech, Santa Cruz, Calif.). The chemiluminescence was detected using an ECL kit according to the instructions of the manufacturer (Amersham Life Science, Arlington Heights, Ill.). For detection of Flag-tagged proteins (e.g., cTnT), anti-Flag M2 antibody (Sigma) was used for immunoblot. For detection of HA-tagged proteins (e.g., wild type ASK1), anti-HA antibody (Roche Diagnostics) was used for immunoblot.

Measuremnent of MgATPase activity in cardiomyocytes: Cardiomyocytes were untreated or treated with H₂O₂ and myofibrils were prepared according to the procedure of Solaro et al. (Solaro R J et al. The purification of cardiac myofibrils with Triton X-100. Biochim Biophys Acta 245:259-62 (1971)). Briefly, cells from a 6-well plate were homogenized in 200 μl of buffer containing 20 mM imidazole HCl (pH 7.0), 1 mM magnesium acetate, 0.1 M KCl, and protease and phosphatase inhibitors (myofibril buffer). Cell pellet was obtained by centrifugation at 750× g for 10 min at 4° C. The pellet was washed three times with myofibril buffer by resuspension followed by centrifugation at 750× g for 10 min. The pellet was then washed twice with myofibril buffer containing 0.1% Triton X-100 and resuspended in 100 μl of myofibril buffer. Purified myofibrils were used for MgATPase activity. Total MgATPase activity was determined at 30° C. in a buffer containing: 20 mM imidozole (pH7.0); 2 mM MgCl₂; 2 mM Na₂ATP; 10 mM NaN₃; 4.86 pM Ca Cl₂ and 50 mM KC1 (MgATPase assay buffer). Basal MgATPase level was determined in the same MgTAPase assay buffer except that 4.86 μM CaCl₂ was replaced by 1.6 mM EGTA. These samples were centrifuged and the phosphate in the protein-free supernatant determined as previously described (Pierce G N et al. Cardiac myofibrillar ATPase activity in diabetic rats. J Mol Cell Cardiol 13:1063-9 (1981)). The difference between the values obtained for total and basal ATPase activities were taken as Ca²⁺-stimulated ATPase activity.

Statistical analysis: Values are expressed as mean ±SD. One-way analysis of variance was used to evaluate differences in MgATPase and myocyte contraction. A level of P<0.05 was accepted as statistically significant.

Results

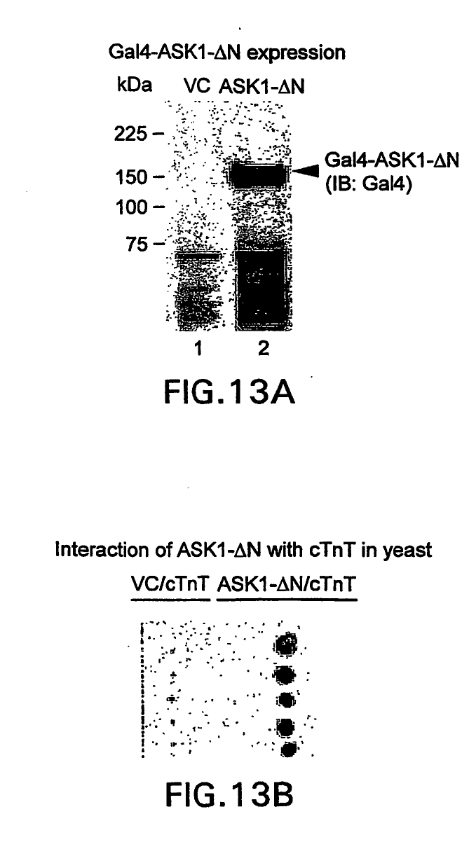

ASK1 binds to cTnT in yeast two-hybrid system: To identify substrates of ASK1 in cardiomyocytes, we used the constitutively active ASK1 (ASK1-ΔN) (Sugden P H et al. “Stress-responsive” mitogen-activated protein kinases (c-Jun N-terminal kinases and p38 mitogen-activated protein kinases) in the myocardium. Circ Res.83:345-52 (1998)) as bait in the yeast two-hybrid system. Among 2×10⁶ transformants screened from a human cardiac library, 10 clones were positive for growth on four-dropout medium (QDO, ade⁻;, leu⁻, trp⁻ and his⁻) and for β-galactosidase assay. Sequence analysis revealed that five of the isolated cDNAs encoded the C-terminal domain (aa 120-288) of human cardiac troponin T (accession No NM_{—}000364). Further experiments using yeast two-hybrid system showed that co-transformation of ASK1-ΔN (FIG. 13), but not the empty vector pAS2.1 (FIG. 13B, VC), with cTnT was positive for growth on QDO and for β-galactosidase activity assay, confirming that cTnT specifically interacted with ASK1-ΔN.

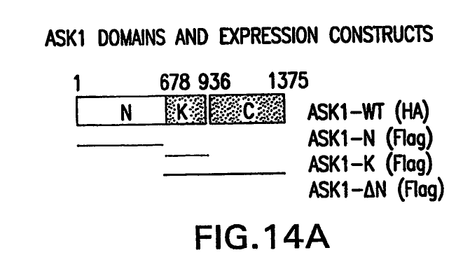

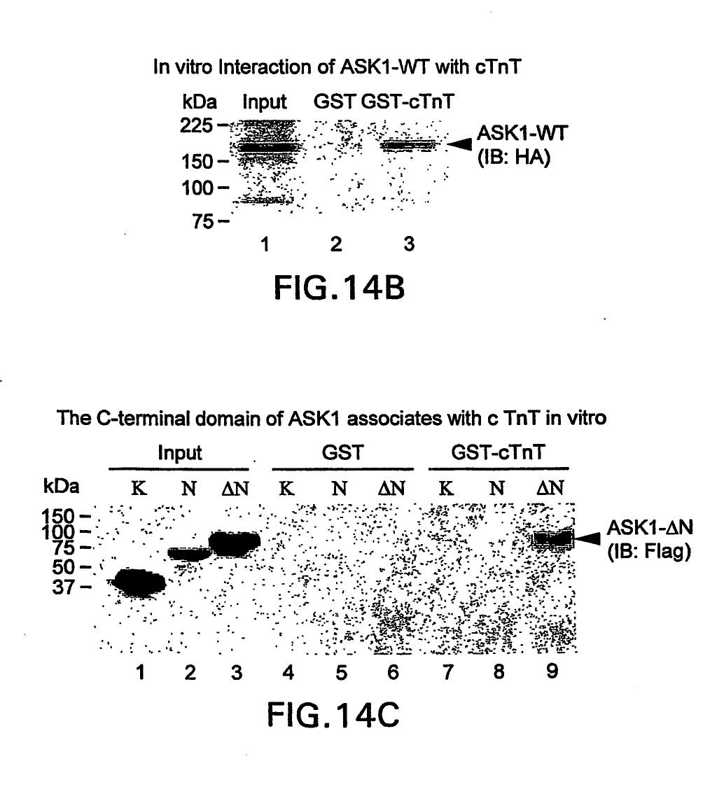

ASK1 associates with cTnT in vitro and in vivo: To identify the region of ASK1 associating with cTnT, expression constructs were made for various ASK1 domains as shown in FIG. 14A—wild type ASK1 (ASK1-WT, aa 1-1375), the N-terminal domain (ASK1-N, aa 1-678), the kinase domain (ASK1-K, aa 678-936) and the C-terminal domain (ASK-ΔN, aa 678-1375). ASK1 expression constructs were transiently transfected in 293T cells. Association between cTnT and different domains of ASK1 was determined using an in vitro GST pull-down assay. Results show that GST-cTnT, but not GST alone, interacted with HA-tagged ASK1-WT (FIG. 14B). Using Flag-tagged ASK1 constructs, GST-cTnT interacted with ASK-ΔN, but not with ASK1-N or ASK1-K (FIG. 14C). These data indicate that the C-terminal domain of ASK1 is responsible for cTnT association. ASK1 did not associate with cTnI by GST pull-down assay.

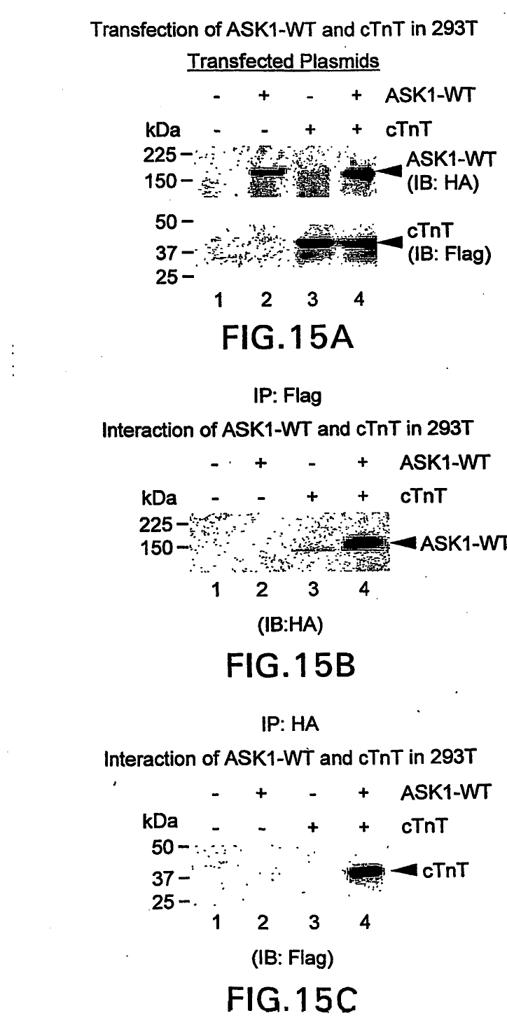

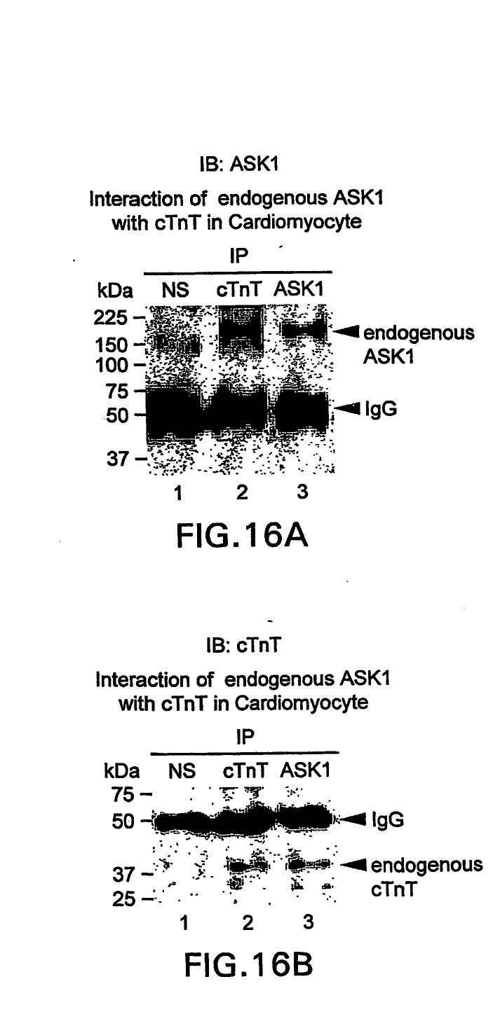

To confirm the interaction of cTnT and ASK1 in intact cells, Flag-cTnT and HA-ASK1 expression plasmids were co-transfected in 293T cells. Expression of cTnT and ASK1 was determined by Western blot with anti-Flag and anti-HA, respectively (FIG. 15A). The association between these two proteins was analyzed by co-immunoprecipitation (IP) assay. FIG. 15B shows that IP with anti-Flag (cTnT) followed by Western blot with anti-HA precipitated ASK1-WT. FIG. 15C shows that IP with anti-HA (ASK1-WT) followed by Western blot with anti-Flag precipitated cTnT. The ability of an antibody against either Flag or HA to precipitate a complex that contains cTnT and ASK1 suggests that cTnT and ASK1 interacts in vivo. To examine interaction of endogenous cTnT and ASK1, rat neonatal cardiomyocytes were isolated and cell lysates were used for IP assay. An antibody against cTnT, but not normal rabbit serum (NS), specifically immunoprecipitated ASK1 (FIG. 16A, lane 2). Conversely, an antibody against ASK1, but not normal rabbit serum, specifically inimunoprecipitated cTnT (FIG. 16B, lane 3), suggesting that endogenous ASK1 and cTnT form a complex in cardiomyocytes.

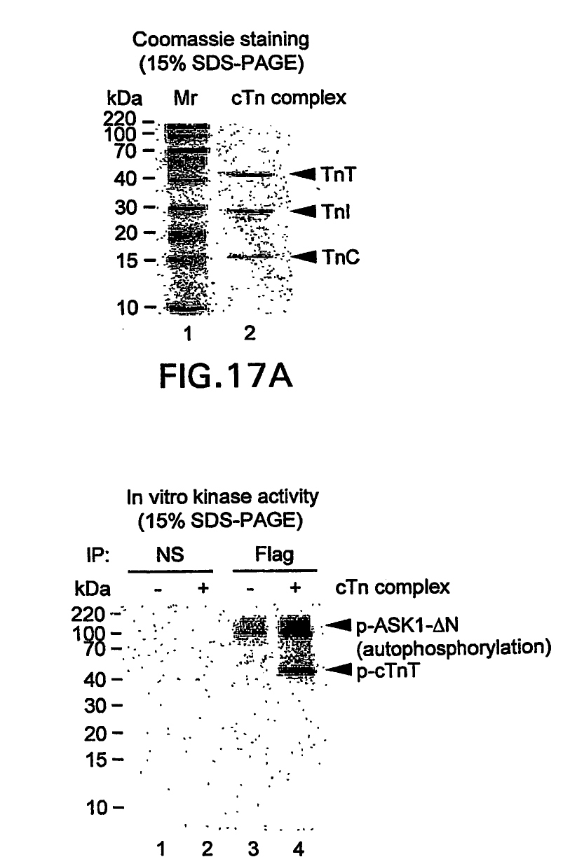

ASK1 phosphorylates cTnT in vitro and in vivo: cTnT is a component of the myofibrillar apparatus which is involved in Ca²⁺-dependent regulation of contraction in cardiac muscles. Phosphorylation of myofilament proteins (including cTnT and cTni) is important in the regulation of contractile activity (Filatov V L et al. Troponin: structure, properties, and mechanism of fimctioning. Biochemistry (Mosc). 64:969-85 (1999); Noland T A, Jr. et al. Protein kinase C phosphorylation of cardiac troponin I and troponin T inhibits Ca(2+)-stimulated MgATPase activity in reconstituted actomyosin and isolated myofibrils, and decreases actin-myosin interactions. J Mol Cell Cardiol. 25:53-65 (1993); Yuasa K et al. A novel interaction of cGMP-dependent protein kinase I with troponin T. J Biol Chem. 274:37429-34 (1999)). The association of ASK1 with cTnT was examined to determine whether such interaction results in phosphorylation of cTnT. First, the phosphorylation of cTnT by ASK1 in vitro was examined. Purified human native cTnT (39 kDa) (FIG. 17A) present in a complex with cTnI (29 kDa) and cTnC (18 kDa) was incubated with Flag-tagged ASK1 -ΔN protein IP with anti-Flag from ASK1-ΔN-expressing 293T cell lysates. Phosphorylation of cTn proteins by ASK1-ΔN was determined using an in vitro kinase assay. Phosphorylated cTnT was visualized by SDS/PAGE followed by autoradiography (FIG. 17B). The identity of cTn proteins was determined by SDS-PAGE followed by Commassie staining (FIG. 17A). Results showed that cTnT, but not cTnI or cTnC, was specifically phosphorylated by immunoprecipitated ASK1-ΔN with anti-Flag (FIG. 17B, lane 4). ASK1-ΔN was autophosphorylated in this assay (FIG. 17B, lanes 3 and 4). Cell lysates immunoprecipitated with normal sera (NS) showed no phosphorylation of cTnT or ASK1-ΔN (FIG. 17B, lane 2).

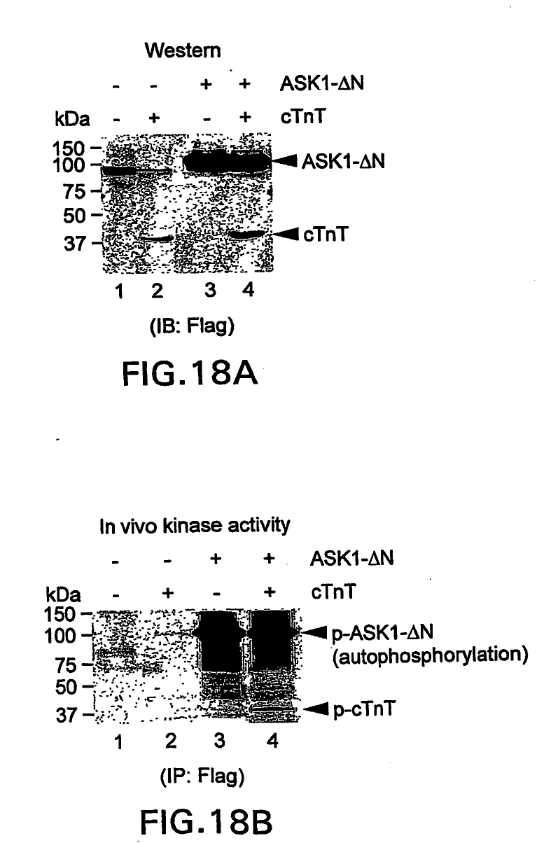

To examine if ASK1 phosphorylates cTnT in vivo, Flag-tagged cTnT and ASK1-ΔN expression plasmids were co-transfected in 293T. Expressions of cTnT and ASK1-ΔN were determined by Western blot with anti-Flag (FIG. 18A). Cells were labeled with ³²P-orthophosphate and phosphorylation of cTnT by ASK1 -ΔN in vivo was determined by IP with anti-Flag followed by SDS-PAGE/autoradiography. FIG. 6B shows that only co-expression of ASK1-ΔN and cTnT (FIG. 18A, lane 4) resulted in phosphorylation of cTnT (FIG. 18B, lane 4). Expression of cTniT alone (FIG. 18B, lane 2) or ASK1-ΔN alone (FIG. 18B, lane 3) did not result in cTnT phosphoprotein, suggesting cTnT was specifically phosphorylated by ASK1-ΔN in vivo. ASK1-ΔN was also autophosphorylated in this assay (FIG. 18B, lanes 3 and 4). Multiple phosphoproteins were present in the in vivo phosphorylation assay compared to the in vitro assay (compare FIG. 17 to FIG. 18B). It is likely that these cellular phosphoproteins are associated with ASK1-ΔN but are not actually phosphorylated by ASK1-ΔN.

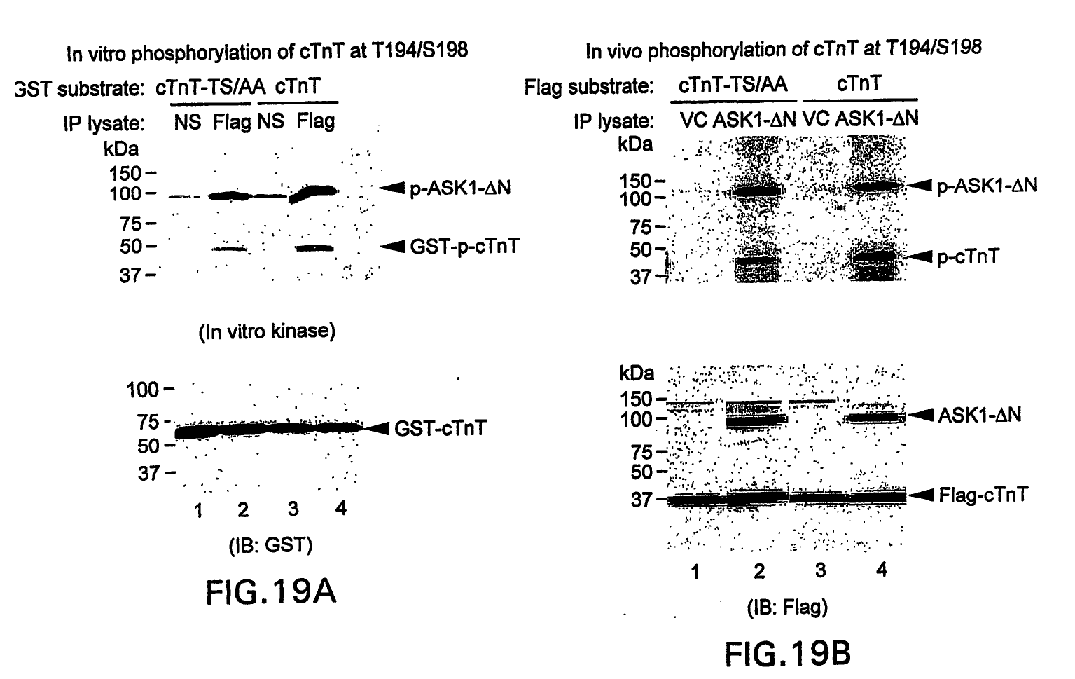

ASK1 phosphorylates cTnT at T194/S198: ASK1 phosphorylates its substrates at a consensus sequence consisting of S/TxxxS/T. There is only one ASK1 phosphorylation consensus sequence in cTnT (T¹⁹⁴ERKS¹⁹⁸) (SEQ ID NO:21). To determine if ASK1 phosphorylates cTnT molecule at this site, a cTnT mutant was constructed with T194A and S198A (“cTnT-TS/AA”) in a mammalian expression Flag-vector and a bacterial expression GST-vector. Phosphorylation of cTnT by immunoprecipitated Flag-ASK1-ΔN was examined in an in vitro kinase assay using purified GST-cTnT or cTnT-TS/AA as substrates. Phosphorylation of cTnT-TS/AA by ASK1-ΔN was decreased compared to wild type cTnT (FIG. 19A). Input proteins were visualized by Western blot with anti-GST and show equal loading of the cTnT substrates (Bottom panel in FIG. 19A). cTnT and mutant cTnT were not phosphorylated by immunoprecipitates with normal sera (NS) (FIG. 19A), indicating that cTnT was specifically phosphorylated by ASK1. We then examined mutant cTnT phosphorylation by ASK1-ΔN in mammalian cells. Flag-tagged wild type cTnT or cTnT-TS/AA were co-expressed with Flag-tagged ASK1-ΔN in 293T cells. Phosphorylation of cTnT was determined by immunoprecipitation with anti-Flag followed by an in vitro kinase assay in the presence of γ-³²P-ATP (FIG. 19B). Phosphorylation of cTnT-TS/AA was decreased compared to wild type (lane 2 vs lane 4 in FIG. 19B). Densitometry analyses showed that mutations at the ASK1 phosphorylation consensus site decreased the extent of cTnT phosphorylation by ˜50%. Western blot with anti-Flag showed equal amounts of expressed proteins (FIG. 19B). These data indicate that T¹⁹⁴ERKS¹⁹⁸ (SEQ ID NO:21) of cTnT are the major phosphorylation sites for ASK1. The presence of residual phosphorylation in cTnT-TS/AA suggests that a second site in cTnT is phosphorylated by ASK1 or ASK1-associated kinase(s) in the immunoprecipitates.

Role of ASK1 in ROS-induced cTnT phosphorylation and contractile dysfunction. The effects of ROS and ASK1 expression on cTnT phosphorylation and cardiac contractility in isolated cardiomyocytes were examined. Neonatal rat cardiomyocytes were treated with H₂O₂ (100 μM) for 1, 5 or 15 min. ASK1 activity was measured by an in vitro kinase assay using GST-MKK4 as a substrate (Liu Y et al. Laminar flow inhibits TNF-induced ASK1 activation by preventing dissociation of ASK1 from its inhibitor 14-3-3. J Clin Invest. 107:917-23 (2001)).

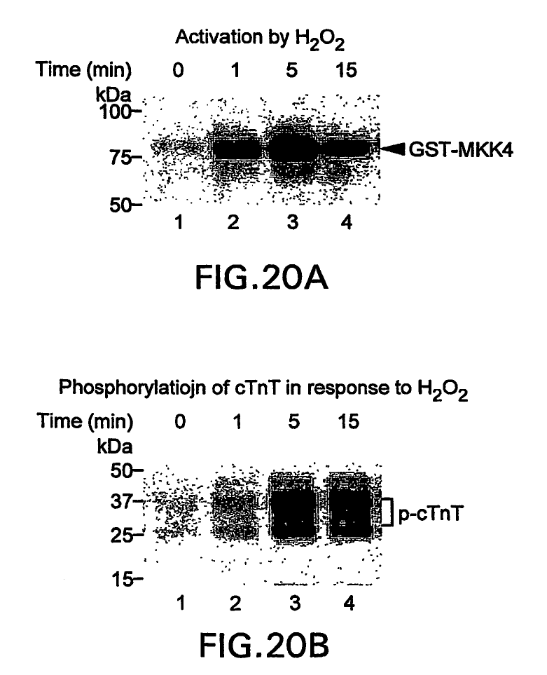

Phosphorylation of endogenous cTnT in cardiomyocytes was examined by in vivo labeling with ³²P-orthophosphate and cell lysates were immunoprecipitated with anti-cTnT followed by SDS-PAGE/autoradiography. Consistent with data from endothelial cells (Liu Y et al. Laminar flow inhibits TNF-induced ASK1 activation by preventing dissociation of ASK1 from its inhibitor 14-3-3. J Clin Invest.107:917-23 (2001)), H₂O₂ induced ASK1 activation in cardiomyocytes at 1 min with peak at 5 min (FIG. 20A). ASK1 activity was associated with a concomitant increase in cTnT phosphorylation with peak at 5 min (FIG. 20B). Cardiomyocyte contraction was determined by microscopy. Each group was performed as duplicates and 3 fields per 6-well dish were examined. Data from three independent experiments showed that spontaneous beating of neonatal cardiomyocytes was completely inhibited at 15 min post-treatment with H₂O₂. Similar kinetics for ASK1 activation and cTnT phosphorylation by H₂O₂ (100 μM) were observed in adult rat cardiomyocytes. Adult cardiac myocytes were field stimulated at 1 Hz and twitch amplitudes were measured in the absence and presence of 100 μM H₂O₂. This concentration of H₂0₂ gradually decayed the twitch amplitudes, and complete inhibition of twitch was observed at 2.38 ±018 min (n=15). The myocytes were rod shaped and visually looked healthy even though the field stimulation failed to produce any twitches. There was no decay of twitch amplitudes in myocytes in absence of H₂O₂ (followed for 15 min). Similar results were obtained from three independent experiments. These data show that ASK1 activation correlates temporally with ROS-induced cTnT phosphorylation and cardiac contractile dysfunction.

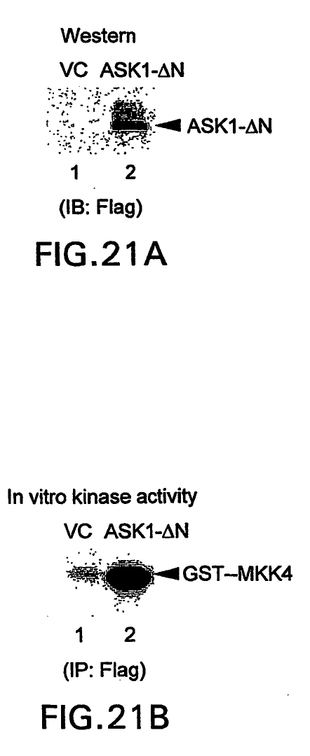

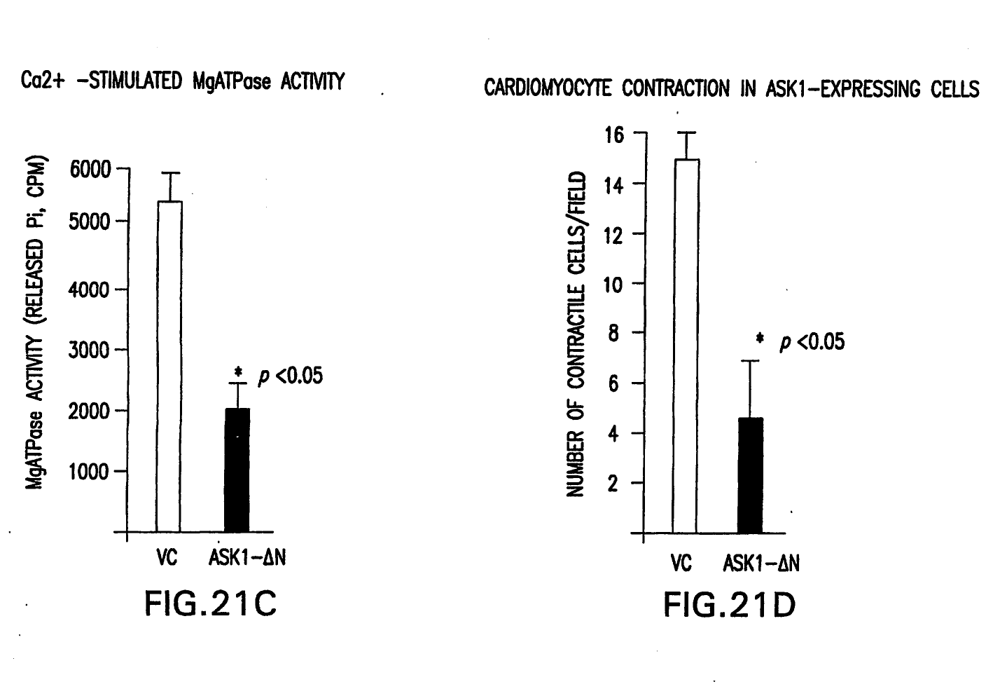

To define roles of ASK1 in ROS-induced contractile dysfunction, the effects of overexpressing ASK1 on Ca²⁺-stimulated MgATPase activity and contractility of cardiomyocytes was examined. A lentiviral expression vector co-expressing ASK1-ΔN and GFP on a bi-cistronic mRNA (HR-ASK1-ΔN-GFP) was constructed, and the lentiviruses were prepared to a titer of 10⁷ pfU/ml. Neonatal rat cardiomyocytes were infected with HR-ASK1-ΔN-GFP or empty vector HR-GFP (VC). Infection of cardiomyocytes by the lentiviral system was confirmed by visualizing GFP under microscope. Results show that cardiomyocytes were effectively infected by the lentiviral system (100% infection with MOI of 50). Expression of ASK1-ΔN was determined by Western blot with anti-Flag (FIG. 21A) and ASK1 activity was determined by an in vitro kinase assay using GST-MKK4 as a substrate (FIG. 21B). MgATPase activity was determined by measuring release of free Pi using ³²P-γ-ATP as a substrate as described. Total MgATPase activity was determined in the assay buffer containing 4.86 μM CaCl₂. Basal MgATPase levels were determined from cardiomyocytes cultured in Ca²⁺-free medium for 2 h (no beating cells were observed under this condition) in the same MgTAPase assay buffer except that 4.86 μM CaCl₂ was replaced by 1.6 mM EGTA. This incubation time in Ca²⁺-free medium did not alter ASK1 expression and activity. Results showed that expression of ASK1-ΔN did not affect the basal MgATPase activity but significantly inhibited Ca²⁺-stimulated MgATPase activity by 60% (FIG. 21C). Cardiomyocyte contractility was determined by counting the contractile GFP-positive cells. Results showed that ASK1-ΔN expression did not decrease the total number of GFP-positive cells even at day 6 post-infection, suggesting that ASK1-ΔN did not induce apoptosis of cardiomyocytes. However, ASK1-ΔN expression decreased the number of contractile GFP-positive cardiomyocytes by 70% (FIG. 21D).

Taken together, these data show ASK1 directly associates with and specifically phosphorylates cardiac TnT leading to inhibition of Ca²⁺-stimulated myosin MgATPase activity. This is important for cardiac contractile dysfimction since H₂O₂-induced ASK1 activation, cTnT phosphorylation and decrease in cardiomyocyte contractility showed similar kinetics. Because ASK1 is critical for ROS/proinflammatory cytokine-induced signaling, the data suggest that ASK1 participates in the pathogenesis of cardiomyopathy and heart failure induced by ROS and cytokines.

It has been shown that phosphorylation of cTnT and cTnI diminishes contractile activity (Noland T A, Jr. et al. Protein kinase C phosphorylation of cardiac troponin I and troponin T inhibits Ca(2+)-stimulated MgATPase activity in reconstituted actomyosin and isolated myofibrils, and decreases actin-myosin interactions. J Mol Cell Cardiol 25:53-65 (1993)). Noland and Kuo showed that exclusive phosphorylation of cTnT results in ˜60% decrease in maximum actomyosin MgATPase activity in fully reconstituted systems. When cTnI was exclusively phosphorylated under the same conditions, only 20% decrease was observed, suggesting that cTnT phosphorylation is important in regulating cardiac contractility.

The present example shows ASK1 is the first identified cTnT kinase. ASK1 directly associates with and specifically phosphorylates cTnT (but not cTnI and cTnC). ASK1 phosphorylates cTnT at sites T194 and S198 within an ASK1 consensus phosphorylation sequence (although other sites may also be phosphorylated). Mutation of this consensus motif ofASK1 phosphorylation sites significantly decreased the extent of cTnT phosphorylation in vivo, indicating that ASK1 is a major kinase responsible for cTnT phosphorylation. This sequence is located at the C-terminus of cTnT, the region which is responsible for association with the C-terminus of cTnC, the N-terminus of cTnI, and the Ca²⁺-dependent interaction with tropomyosin. It is conceivable that interaction with ASK1 and subsequent phosphorylation by ASK1 may significantly alter interactions of cTnT with other contractile components leading to contractile dysfunction.

Overexpression of ASK1 inhibits MgATPase and contractility in cardiomyocytes, demonstrating a novel function of ASK1 in regulating cardiac function. This role is supported by the observation that transgenic mice in which the constitutively active ASK1 is expressed in the heart (α-myosin heavy chain promoter) die before birth. It has not been determined whether lethality of ASK1-transgenic mice is due to ASK1-induced contractile dysfimction or apoptosis. However, these two events may be functionally linked in ROS/cytokine-induced cardiac pathogenesis.

In summary, these data define a role for ASK1 in ROS-induced cardiac contractile dysfunction. While the roles of ASK1 in heart function have been studied to a limited extent, this study suggests that inhibiting ASK1 activity may provide a valid approach for treatment of cardiovascular diseases.

Any patents or publications mentioned in the specification are indicative of the level of those skilled in the art to which the invention pertains. These patents and publications are herein incorporated by reference to the same extent as if each individual publication was specifically and individually indicated to be incorporated by reference.

The present invention is not limited in scope by the above-referenced deposit or the embodiments disclosed in the examples which are intended as illustrations of a few aspects of the invention and any embodiments which are functionally equivalent are within the scope of this invention. Various modifications of the invention in addition to those shown and described herein will become apparent to those skilled in the art and are intended to fall within the scope of the appended claims.

### REFERENCES

1. Abrahmsen L. et al., Biochemistry, 30:4151 (1991).
2. Altschul et al., J. Mol. Biol. 215:403-410 (1990).
3. Altschul et al., Nucl. Acids Res. 25: 3389-3402 (1997).
4. Amersham Life Science Proximity News. Issue 17 (1995).
5. Amersham Life Science Proximity News. Issue 18 (1995).
6. Ausubel et al. (eds.), Current Protocols in Molecular Biology, (John Wiley & Sons, NY) (1995).
7. Ausubel F. et al., Current Protocols in Molecular Biology, John Wiley & Sons, New York, NY, (1998).
8. Ausubel F. et al., Current Protocols in Molecular Biology, John Wiley & Sons, New York, N.Y. (1998).
9. Baggiolini M. et al., FEBS Lett. 307:97-101 (1992).
10. Barge, S. M. et al., Pharmaceutical Salts, J. Pharm. Sci 66:1-19 (1977).
11. Berkner et al., J. Virology 61:1213-1220 (1987).
12. Bicamumpaka C. et al., Development of a fluorescence polarization immunoassay (FPIA) for the quantitative determination of paclitaxel. J. Immunol. Methods 212:1-7 (1998).
13. Bioreversible Carriers in Drug Design, ed. Edward B. Roche, American Pharmaceutical Association and Pergamon Press (1987).
14. Bodansky M. et al., Ed. Principles of Peptide Synthesis. Springer-Verlag Inc., NY (1993).
15. Boemer et al., J. Immunol. 147(1):86-95 (1991).
16. Bout, Human Gene Therapy 5:3-10 (1994).
17. Brodeur et al., Monoclonal Antibody Production Techniques and Applications Marcel Dekker, Inc., New York, pp. 51-63 (1987).
18. Brown et al., J. Virology 12:386-396 (1973).
19. Bruggemann. et aL, Year in Immuno. 7:33 (1993).
20. Brummelkamp, TR. et al. A System for Stable Expression of Short Interfering RNAs in Mammalian Cells. Science 296:550-553 (2002).
21. Caillaud, Eur. J. Neuroscience 5:1287-1291 (1993).
22. Carter et al., Proc. Natl. Acad. Sci. USA, 89:4285 (1992).
23. Chang H Y. et al. Activation of apoptosis signal-regulating kinase 1 (ASK1) by the adapter protein Daxx. Science. 281:1860-3 (1998).
24. Chardonnet et al., Virology 40:462-477 (1970).
25. Chothia et al., J. Mol. Biol., 196:901 (1987).
26. Clark-Lewis I. et al., Biochemistry, 30:3128 (1991).
27. Clark-Lewis I. et al., J. Biol.Chem., 269:16075 (1994).
28. Cole et al., Monoclonal Antibodies and Cancer Therapy, Alan R. Liss, p. 77 (1985).
29. Cotter et al., Curr Opin Mol Ther 5: 633-644, (1999).
30. Creighton, T. E., Proteins: Structure and Molecular Properties, W. H. Freeman & Co., San Francisco pp 79-86 (1983).
31. Davidson et al., J. Virology 61:1226-1239 (1987).
32. Davis R J., Signal transduction by the JNK group of MAP kinases. Cell 103:239-52 (2000).
33. Dawson et al., Synthesis of Proteins by Native Chemical Ligation. Science 266:776-779 (1994).
34. deLisle Milton R C. et al., Techniques in Protein Chemistry IV. Academic Press, New York, pp. 257-267 (1992).
35. Fagerstam L G. et al., Biospecific interaction analysis using surface plasmon resonance detection applied to kinetic, binding site and concentration analysis. Chromatography 597:397-410 (1992).
36. Filatov V L. et al., Troponin: structure, properties, and mechanism of functioning. Biochemistry (Mosc). 64:969-85 (1999).
37. Fisher, Laboratory Techniques In Biochemistry And Molecular Biology, Work and Burdon, Eds., Elsevier (1980).
38. Garcia-Cardena G. et al., Dynamic activation of endothelial nitric oxide synthase by Hsp90. Nature. 392: 821-823 (1998).
39. Goding, Monoclonal Antibodies: Principles and Practice Academic Press, pp. 59-103 (1986).
40. Gomez-Foix, J. Biol. Chem. 267:25129-25134 (1992).
41. Gotoh Y. et al., Reactive oxygen species- and dimerization-induced activation of apoptosis signal-regulating kinase 1 in tumor necrosis factor-alpha signal transduction. J Biol Chem. 273:17477-82 (1998).
42. Green D R., Apoptotic pathways: paper wraps stone blunts scissors. Cell. 102:1-4 (2000).
43. Guzman, Circulation Research 73:1201-1207 (1993).
44. Haimovitz-Friedman A. et al., Lipopolysaccharide induces disseminated endothelial apoptosis requiring ceramide generation. J Exp Med. 186:1831-1841 (1997).
45. Haj-Ahmad et al., J. Virology 57:267-274 (1986).
46. Hannon, G J., RNA interference-Nature 418:244-251 (2002).
47. Harlow and Lane, Antibodies, A Laboratory Manual, Cold Spring Harbor Publications, New York, (1988).
48. Hatai T. et al., Execution of apoptosis signal-regulating kinase 1 (ASK1)-induced apoptosis by the mitochondria-dependent caspase activation. J Biol Chem. 275:26576-81 (2000).
49. Hengartner M O., The biochemistry of apoptosis. Nature. 407:770-6 (2000).
50. Higuchi T. et al., Pro-drugs as Novel Delivery Systems. Vol. 14 of the A.C.S. Symposium Series.
51. Holmgren A., Thioredoxin and glutaredoxin systems. J Biol Chem. 264:13963-6 (1989).
52. Holmgren A., Thioredoxin. Annu Rev Biochem. 54:237-71 (1985).
53. Hoogenboom et al., J. Mol. Biol. 227:381 (1991).
54. Ichijo H. et al., Induction of apoptosis by ASK1, a mammalian MAPKKK that activates SAPK/JNK and p38 signaling pathways. Science. 275:90-4 (1997).
55. Ichijo H., From receptors to stress-activated MAP kinases. Oncogene. 18:6087-93 (1999).
56. Ip Y T. et al., Signal transduction by the c-Jun N-terminal kinase (JNK)—from inflammation to development. Curr Opin Cell Biol. 10:205-19 (1998).
57. Jakobovits et al., Analysis of homozygous mutant chimeric mice: deletion of the immunoglobulin heavy-chain joining region blocks B-cell development and antibody production. Proc. Natl. Acad. Sci. USA. 90:2551-255 (1993).
58. Jakobovits et al., Germ-line transmission and expression of a human-derived yeast artificial chromosome. Nature 362:255-258 (1993).
59. Jiskoot W. et al., Preparation and application of a fluorescein-labeled peptide for determining the affinity constant of a monoclonal antibody-hapten complex by fluorescence polarization. Anal Biochem. 196:421-26. (1991).
60. Jolley M E., Fluorescence polarization immunoassay for the determination of therapeutic drug levels in human plasma. J Anal Toxicol. 5:236-40. (1981).
61. Jones PT. et al., Replacing the complementarity-determining regions in a human antibody with those from a mouse. Nature 321:522-525 (1986).
62. Kabat E. A. et al., Sequences of Proteins of Immunological Interest. National Institutes of Health, Bethesda, Md. (1987).
63. Karlin and Altschul, Proc. Natl. Acad. Sci. USA 87:2264-2268 (1990).
64. Kirshenbaum, J. Clin. Invest. 92:381-387 (1993).
65. Kohler and Milstein, Nature 256:495 (1975).
66. Kozbor J., Immunol. 133:3001 (1984).
67. La Salle, Science 259:988-990 (1993).
68. Laney J D. et al., Substrate targeting in the ubiquitin system. Cell. 97:427-30 (1999).
69. Liu H. et al., Activation of apoptosis signal-regulating kinase 1 (ASK1 ) by tumor necrosis factor receptor-associated factor 2 requires prior dissociation of the ASK1 inhibitor thioredoxin. Mol Cell Biol. 20:2198-208 (2000).
70. Liu Y. et al., Laminar flow inhibits TNF-induced ASK1 activation by preventing dissociation of ASK1 from its inhibitor 14-3-3. J Clin. Invest. 107:917-23 (2001).
71. Liu Y. et al., Thioredoxin promotes ASK1 ubiquitination and degradation to inhibit ASK1-mediated apoptosis in a redox activity-independent manner. Cir. Res. 90: In press (2002).
72. Liu, Y. et al., Flow inhibits TNF-induced ASK1 activation by enhancing interactions of ASK1 with its inhibitor 14-3-3. J Clin. Invest. 107, 917-923 (2001).
73. Lonberg et al., Human antibodies from transgenic mice, Int. Rev. Immunol. 13:65-93 (1995).
74. Marks et al., J. Mol. Biol. 222:581 (1991).
75. Massie et al., Mol. Cell. Biol. 6:2872-2883 (1986).
76. Min W. et al., TNF initiates E-selection transcription in human endothelial cells through parallel TRAF-NF-κB and TRAF-RAC/CDC42-JNK-ATF2/c-Jun pathways. J. Inmunol. 159, 3508-18 (1997).
77. Morrison L E., Time-resolved detection of energy transfer:theory and application to immunoassays. Anal. Biochem.174:101-120 (1988).
78. Morrison S L. et al., Chimeric human antibody molecules: mouse antigen-binding domains with human constant region domains Proc. Natl. Acad. Sci. USA. 81:6851-6855 (1984).
79. Morsy, J. Clin. Invest. 92:1580-1586 (1993).
80. Moullier, Nature Genetics 4:154-159 (1993).
81. Moulton, K S. et al., Angiogenesis inhibitors endostatin or TNP-470 reduce intimal neovascularization and plaque growth in apolipoprotein E-deficient mice. Circulation. 99: 1726-1732. (1999).
82. Mulligan, Science 260:926-932 (1993).
83. Munson et aL, Anal. Biochem. 107:220 (1980).
84. Naldini, L. et al., Efficient transfer, integration, and sustained long-term expression of the transgene in adult rat brains injected with a lentiviral vector. Proc. Natl. Acad. Sci. USA. 93:11382-11388 (1996).
85. Nishitoh, H. et al., ASK1 is essential for JNK/SAPK activation by TRAF2. Mol Cell. 2:389-95 (1998).
86. Noland T A, Jr. et al., Protein kinase C phosphorylation of cardiac troponin I and troponin T inhibits Ca(2+)-stimulated MgATPase activity in reconstituted actomyosin and isolated myofibrils, and decreases actin-myosin interactions. J Mol Cell Cardiol. 25:53-65 (1993).
87. Park, Y W. et al., Homogenouse Proximity tyrosine kinasee assays:scintillation proximity assay versus homogenous time-resolved fluorescence. Anal. Biochem. 269:94-104 (1999).
88. Paul, C P. et al., Effective expression of small interfering RNA in human cells. Nature Biotech. 20:505-508 (2002).
89. Pierce, G N. et al., Cardiac myofibrillar ATPase activity in diabetic rats. J Mol Cell Cardiol. 13:1063-9 (1981).
90. Poppa, V. et al., Endothelial NO synthase is increased in regenerating endothelium after denuding injury of the rat aorta. Arterioscler Thromb Vasc Biol. 18:1312-21 (1998).
91. Pouwels et al., Cloning Vectors: A Laboratory Manual, 1985, Supp. (1987).
92. Powis G. et al., The role of the redox protein thioredoxin in cell growth and cancer. Free Radic Biol Med. 29:312-22 (2000).
93. Presta et al., J. Immunol. 151:2623 (1993).
94. Presta, Curr. Op. Struct. Biol. 2:593-596 (1992).
95. Ragot, J., Gen. Virology 74:501-507 (1993).
96. Rajarathnam, K. et al., Biochemistry 33:6623-30 (1994).
97. Ram et al., Cancer Res. 53:83-88, (1993).
98. Rich, Human Gene Therapy 4:461-476 (1993).
99. Riechmann et al., Nature, 332:323-327 (1988).
100. Roessler, J. Clin. Invest. 92:1085-1092 (1993).
101. Saitoh, M., Mammalian thioredoxin is a direct inhibitor of apoptosis signal- regulating kinase (ASK) 1. Embo J. 17:2596-606 (1998).
102. Sambrook et al., , Molecular Cloning, A Laboratory Manual, Cold Spring Harbor Press, NY. (1989).
103. Sayama K. et al., Apoptosis signal-regulating kinase 1 (ASK1 ) is an intracellular inducer of keratinocyte differentiation. J Biol Chem. 276:999-1004 (2001).
104. Schnolzer, M. et al., Science 256:221 (1992).
105. Seethala, R. et al., A fluorescence polarization competition immunoassay for tyrosine kinases. Anal. Biochem. 255:257-62. (1998).
106. Seth et al., J. Virol. 51:650-655 (1984).
107. Seth et al., Mol. Cell. Biol. 4:1528-1533 (1984).
b 108. Sheu S S., Measurement of cytosolic free calcium concentration in isolated rat ventricular myocytes with quin 2. Circ Res. 55:830-4 (1984).
109. Simpson P. et al., Differentiation of rat myocytes in single cell cultures with and without proliferating nomnyocardial cells. Cross-striations, ultrastructure, and chronotropic response to isoproterenol. Circ Res. 50:101-16 (1982)
110. Sims et al., J. Inmunol. 151:2296(1993).
111. Slowik MR. et al., Evidence that tumor necrosis factor triggers apoptosis in human endothelial cells by interleukin-1-converting enzyme-like protease-dependent and -independent pathways. Lab Invest. 77:257-67 (1997).
112. Solaro RJ. et al., The purification of cardiac myofibrils with Triton X-100. Biochim Biophys Acta. 245:259-62 (197).
113. Stenroos, K. et al., Homogeneous rime-resolved IL-2-IL-2RRc Assay using fluorescence resonance energy transfer. Cytokine 1 100(7):495-499 (1988).
114. Sugden PH. et al., “Stress-responsive” mitogen-activated protein kinases (c-Jun N-terminal kinases and p38 mitogen-activated protein kinases) in the myocardium. Circ Res.83:345-52 (1998).
115. Sun et al., Nature genetics 8: 33-41 (1994).
116. Svensson et al., J. Virology 55:442-449 (1985).
117. Synthetic Peptides: A User Guide. W.H. Freeman and Co., N.Y. (1992).
118. Takeda K. et al., Apoptosis signal-regulating kinase 1 (ASK1 ) induces neuronal differentiation and survival of PC12 cells.
119. Tobiume K. et al., ASK1 is required for sustained activations ofJNK/p38 MAP kinases and apoptosis. EMBO Rep. 2:222-8 (2001).
120. Toumier C. et al., Requirement of JNK for stress-induced activation of the cytochrome c-mediated death pathway. Science. 288:870-4 (2000)
121. Treier et al., Ubiquitin-dependent c-Jun degradation in vivo is mediated by the delta domain. Cell. 78:787-98 (1994).
122. Varga et al., J. Virology 65:6061-6070 (1991).
123. Verhoeyen et al., Science 239:1534-1536 (1988).
124. Verma, I. M., Retroviral vectors for gene transfer. In Microbiology-1985, American Society for Microbiology, pp. 229-232, Washington (1985).
125. Wickham et al., Cell 73:309-319 (1993).
126. Yin G. et al., Endostatin gene transfer inhibits joint angiogenesis and pannus formation in inflammatory arthritis. Molecular Therapy. 5:547-554 (2002).
127. Yuasa K. et al., A novel interaction of cGMP-dependent protein kinase I with troponin T. J Biol Chem. 274:37429-34 (1999).
128. Zabner, Cell 75:207-216 (1993).
129. Zabner, Nature Genetics 6:75-83 (1994).
130. Zhang L. et al., Suppression of apoptosis signal-regulating kinase 1-induced cell death by 14-3-3 proteins. Proc Natl Acad Sci U S A. 96:8511-5 (1999).
131. Zhang, Generation and identification of recombinant adenovirus by liposome-mediated transfection and PCR analysis. BioTechniques 15:868-872 (1993).
132. Zoller, M J. et al., Nucl. Acids Res. 10:6487-500 (1982).

What is claimed is:

1. A mutant thioredoxin molecule, wherein the thioredoxin molecule is resistant to the oxidizing effects of cytokines or reactive oxygen species.

2. The mutant thioredoxin molecule of claim 1, comprising an amino acid alteration that decreases the number of sulfhydryl groups in the molecule as compared to wild type thioredoxin.

3. The mutant thioredoxin molecule of claim 1, wherein the amino acid alteration is a cysteine substitution or deletion.

4. The mutant thioredoxin molecule of claim 2, wherein the substitution or deletion is at residue 32 or an analogous residue.

5. The mutant thioredoin molecule of claim 4, further comprising a substitution or deletion at residue 69 or an analogous residue.

6. The mutant thioredoxin molecule of claim 2, wherein the substitution or deletion is at residue 35 or an analogous residue.

7. A mutant thioredoxin molecule, wherein the thioredoxin molecule is resistant to S-nitrosylation of a SH-group by nitrous oxide.

8. The mutant thioredoxin molecule of claim 7, comprising an amino acid alteration that decreases the number of sulfhydryl groups in the molecule as compared to the wild type thioredoxin.

9. The mutant thioredoxin molecule of claim 8, wherein the amino acid alteration is a substitution or deletion of a cysteine.

10. The mutant thioredoxin molecule of claim 9, wherein the substitution or deletion is at residue 69 or an analogous residue.

11. A method of decreasing, in a target tissue, inflammation induced by a reactive oxygen species, comprising contacting the target tissue with the mutant thioredoxin molecule of claim 1.

12. A method of decreasing, in a target tissue, cytokine-induced inflammation, comprising contacting the target tissue with the mutant thioredoxin molecule of claim 1.

13. A method of decreasing cardiac muscle contractile dysfunction, comprising contacting cardiac myocytes with the mutant thioredoxin molecule of claim 1.

14. The method of claim 13, wherein the cardiac muscle dysfimction is induced by cytokines or reactive oxygen species.

15. A method of decreasing apoptosis in a target tissue, comprising contacting the target tissue with the mutant thioredoxin molecule of claim 1.

16. A method of decreasing insulin resistance in a target tissue, comprising contacting the target tissue with the mutant thioredoxin molecule of claim 1.

17. A method of decreasing cytokine-induced or reactive oxygen species-induced inflammation, insulin resistance, cardiac muscle contractile dysfunction, or apoptosis in a target tissue, comprising contacting the target tissue with a small molecule that blocks a thiol group of a thioredoxin molecule.

18. A method of treating a subject with atherosclerosis or a subject at risk of atherosclerosis, comprising administering to the subject an agent that inactivates endothelial cell dysfunction mediated by a redox-regulated pathway.

19. The method of claim 18 wherein the agent is a mutant thioredoxin molecule, wherein the thioredoxin molecule is resistant to the oxidizing effects of cytokines or reactive oxygen species.

20. The method of claim 18, wherein the redox-regulated pathway is the ASK1 pathway.

21. The method of claim 18, wherein the redox-regulated pathway is the NF-κB pathway.

22. The method of claim 18, wherein the redox-regulated pathway is the p53 pathway.

23. A method of treating a subject with diabetes or a subject at risk for diabetes, comprising administering to the subject an agent that inactivates insulin resistance mediated by a redox-regulated pathway.

24. The method of claim 23, wherein the diabetes is Type 2 diabetes.

25. The method of claim 23, wherein the agent is a mutant thioredoxin molecule, wherein the thioredoxin molecule is resistant to the oxidizing effects of cytokines or reactive oxygen species.

26. The method of claim 23, wherein the redox-regulated pathway is the ASK1 pathway.

27. The method of claim 23, wherein the redox-regulated pathway is the NF-κB pathway.

28. The method of claim 23, wherein the redox-regulated pathway is the p53 pathway.

29. A method of treating a subject with an apoptotic disease or at risk for an apoptotic disease, comprising administering to the subject an agent that inactivates apoptosis mediated by a redox-regulated pathway.

30. The method of claim 29, wherein the apoptotic disease is a neurodegenerative disease.

31. The method of claim 29, wherein the agent is a mutant thioredoxin molecule, wherein the thioredoxin molecule is resistant to the oxidizing effects of cytokines or reactive oxygen species.

32. The method of claim 29, wherein the redox-regulated pathway is the ASK1 pathway.

33. The method of claim 29, wherein the redox-regulated pathway is the NF-κB pathway.

34. The method of claim 29, wherein the redox-regulated pathway is the p53 pathway.

35. A method of treating a subject with cardiac dysfunction or a subject at risk of cardiac dysfunction, comprising administering to the subject an agent that inactivates cardiac dysfunction mediated by a redox-regulated pathway.

36. The method of claim 35, wherein the cardiac dysfunction is selected from the group consisting of myocardial infarction, cardiomyopathy, arterial hypertension, and heart failure.

37. The method of claim 35, wherein the agent is a mutant thioredoxin molecule, wherein the thioredoxin molecule is resistant to the oxidizing effects of cytokines or reactive oxygen species.

38. The method of claim 35, wherein the redox-regulated pathway is the ASK1 pathway.

39. The method of claim 35, wherein the redox-regulated pathway is the NF-κB pathway.

40. The method of claim 35, wherein the redox-regulated pathway is the p53 pathway.

41. A method of treating a subject with an angiogenesis-dependent disease or a subject at risk for an angiogenesis dependent disease, comprising administering to the subject an agent that blocks thioredoxin-inactivation of apoptosis, wherein the apoptosis is mediated by a redox-regulated pathway.

42. The method of claim 41, wherein the angiogenesis dependent disease is an inflammatory disease.

43. The method of claim 42, wherein the inflammatory disease is rheumatoid arthritis.

44. The method of claim 41, wherein the angiogenesis dependent disease is cancer.

45. The method of claim 41, wherein the agent oxidizes thioredoxin.

46. The method of claim 41, wherein the redox-regulated pathway is the ASK1 pathway.

47. The method of claim 41, wherein the redox-regulated pathway is the NF-κB pathway.

48. The method of claim 41, wherein the redox-regulated pathway is the p53 pathway.

49. A method of diagnosing an angiogenesis dependent disease in a subject or of identifying a subject at risk for developing the angiogenesis dependent disease, comprising detecting, in a biological sample of the subject, levels of reduced thioredoxin, wherein the angiogenesis dependent disease is indicated by an elevated level of reduced thioredoxin as compared to control levels.

50. The method of claim 49, wherein the angiogenesis dependent disease is cancer.

51. The method of claim 49, wherein the angiogenesis dependent disease is arthritis.

52. A method of diagnosing an apoptotic disease in a subject or of identifying a subject at risk for developing the apoptotic disease, comprising detecting, in a biological sample of the subject, levels of oxidized thioredoxin, wherein the apoptotic disease is indicated by an elevated level of oxidized thioredoxin as compared to control levels.

53. The method of claim 52, wherein the apoptotic disease is a neurodegenerative disease.

54. A method of screening a subject for a genetic risk of an angiogenesis dependent disease, comprising detecting a gene that encodes a mutant thioredoxin molecule.

55. The method of claim 54, wherein the mutant thioredoxin molecule is resistant to the oxidizing effects of cytokines or reactive oxygen species.

56. The method of claim 55, wherein the mutant thioredoxin molecule comprises an amino acid alteration that decreases the number of sulfhydryl groups in the molecule as compared to wild type thioredoxin.

57. The method of claim 56, wherein the amino acid alteration is a cysteine to serine substitution.

58. The method of claim 57, wherein the substitution is at residue 32.

59. The method of claim 57, wherein the substitution is at residue 35.

60. A method of screening a subject for a genetic risk of an apoptotic disease, comprising detecting a gene that encodes a mutant thioredoxin molecule.

61. The method of claim 60, wherein the mutant thioredoxin molecule is resistant to S-nitrosylation of a SH-group by nitrous oxide.

62. The method of claim 61, wherein the mutant thioredoxin molecule comprises an amino acid alteration that decreases the number of sulfhydryl groups in the molecule as compared to wild type thioredoxin.

63. The method of claim 63, wherein the amino acid alteration is a substitution or deletion of a cysteine.

64. The method of claim 63, wherein the substitution or deletion is at residue 69.


SEQUENCE LISTING (58 sequences)

SEQ ID NO: 1 - protein, 105 aa, Artificial Sequence
  Description of Artificial Sequence/note = synthetic construct
MVKQIESKTAFQEALDAAGDKLVVVDFSATWCGPCKMIKPFFHSLSEKYSNVIFLEVDVDDCQDVASECEVKCMPTFQFFKKGQKVGEFSGANKEKLEATINELV

SEQ ID NO: 2 - protein, 105 aa, Artificial Sequence
  Description of Artificial Sequence/note = synthetic construct
MVKQIESKTAFQEALDAAGDKLVVVDFSATWSGPCKMIKPFFHSLSEKYSNVIFLEVDVDDCQDVASECEVKCMPTFQFFKKGQKVGEFSGANKEKLEATINELV

SEQ ID NO: 3 - protein, 105 aa, Artificial Sequence
  Description of Artificial Sequence/note = synthetic construct
MVKQIESKTAFQEALDAAGDKLVVVDFSATWCGPSKMIKPFFHSLSEKYSNVIFLEVDVDDCQDVASECEVKCMPTFQFFKKGQKVGEFSGANKEKLEATINELV

SEQ ID NO: 4 - DNA, 318 bp, Artificial Sequence
  Description of Artificial Sequence/note = synthetic construct
atggtgaagcagatcgagagcaagactgcttttcaggaagccttggacgctgcaggtgataaacttgtagtagttgacttctcagccacgtggtgtgggccttgcaaaatgatcaagcctttctttcattccctctctgaaaagtattccaacgtgatattccttgaagtagatgtggatgactgtcaggatgttgcttcagagtgtgaagtcaaatgcatgccaacattccagttttttaagaagggacaaaaggtgggtgaattttctggagccaataaggaaaagcttgaagccaccattaatgaattagtctaa

SEQ ID NO: 5 - DNA, 318 bp, Artificial Sequence
  Description of Artificial Sequence/note = synthetic construct
atggtgaagcagatcgagagcaagactgcttttcaggaagccttggacgctgcaggtgataaacttgtagtagttgacttctcagccacgtggcgtgggccttgcaaaatgatcaagcctttctttcattccctctctgaaaagtattccaacgtgatattccttgaagtagatgtggatgactgtcaggatgttgcttcagagtgtgaagtcaaatgcatgccaacattccagttttttaagaagggacaaaaggtgggtgaattttctggagccaataaggaaaagcttgaagccaccattaatgaattagtctaa

SEQ ID NO: 6 - DNA, 318 bp, Artificial Sequence
  Description of Artificial Sequence/note = synthetic construct
atggtgaagcagatcgagagcaagactgcttttcaggaagccttggacgctgcaggtgataaacttgtagtagttgacttctcagccacgtggtgtgggcctcgcaaaatgatcaagcctttctttcattccctctctgaaaagtattccaacgtgatattccttgaagtagatgtggatgactgtcaggatgttgcttcagagtgtgaagtcaaatgcatgccaacattccagttttttaagaagggacaaaaggtgggtgaattttctggagccaataaggaaaagcttgaagccaccattaatgaattagtctaa

SEQ ID NO: 7 - DNA, 24 bp, Artificial Sequence
  Description of Artificial Sequence/note = synthetic construct
aagcttatggtgaagcagatcgag

SEQ ID NO: 8 - DNA, 24 bp, Artificial Sequence
  Description of Artificial Sequence/note = synthetic construct
ctcgagttagactaattcattaat

SEQ ID NO: 9 - protein, 165 aa, Artificial Sequence
  Description of Artificial Sequence/note = synthetic construct
MAQRLLLRRFLASVISRKPSQGQWPPLTSRALQTPQCSPGGLTVTPNPARTIYTTRISLTTFNIQDGPDFQDRVVNSETPVVVDFHAQWCGPCKILGPRLEMVAKQHGKVVMAKVDIDDHTDLAIEYEVSAVPTVLAMKNGDVVDKFVGIKDEDQLEAFLKKLIG

SEQ ID NO: 10 - protein, 165 aa, Artificial Sequence
  Description of Artificial Sequence/note = synthetic construct
MAQRLLLRRFLASVISRKPSQGQWPPLTSRALQTPQCSPGGLTVTPNPARTIYTTRISLTTFNIQDGPDFQDRVVNSETPVVVDFHAQWSGPCKILGPRLEMVAKQHGKVVMAKVDIDDHTDLAIEYEVSAVPTVLAMKNGDVVDKFVGIKDEDQLEAFLKKLIG

SEQ ID NO: 11 - protein, 165 aa, Artificial Sequence
  Description of Artificial Sequence/note = synthetic construct
MAQRLLLRRFLASVISRKPSQGQWPPLTSRALQTPQCSPGGLTVTPNPARTIYTTRISLTTFNIQDGPDFQDRVVNSETPVVVDFHAQWCGPSKILGPRLEMVAKQHGKVVMAKVDIDDHTDLAIEYEVSAVPTVLAMKNGDVVDKFVGIKDEDQLEAFLKKLIG

SEQ ID NO: 12 - DNA, 502 bp, Artificial Sequence
  Description of Artificial Sequence/note = synthetic construct
atggctcagcgacttcttctgaggaggttcctggcctctgtcatctccaggaagccctctcagggtcagtggccacccctcacttccagagccctgcagaccccacaatgcagtcctggtggcctgactgtaacacccaacccagcccggacaatatacaccacgaggatctccttgacaacctttaatatccaggatggacctgactttcaagaccgagtggtcaacagtgagacaccagtggttgtggatttccacgcacagtggtgtggaccctgcaagatcctggggccgaggttagagaagatggtggccaagcagcacgggaaggtggtgatggccaaggtggatattgatgaccacacagacctcgccattgagtatgaggtgtcagcggtgcccactgtgctggccatgaagaatggggacgtggtggacaagtttgtgggcatcaaggatgaggatcagttggaggccttcctgaagaagctgattggctgac

SEQ ID NO: 13 - DNA, 502 bp, Artificial Sequence
  Description of Artificial Sequence/note = synthetic construct
atggctcagcgacttcttctgaggaggttcctggcctctgtcatctccaggaagccctctcagggtcagtggccacccctcacttccagagccctgcagaccccacaatgcagtcctggtggcctgactgtaacacccaacccagcccggacaatatacaccacgaggatctccttgacaacctttaatatccaggatggacctgactttcaagaccgagtggtcaacagtgagacaccagtggttgtggatttccacgcacagtggagtggaccctgcaagatcctggggccgaggttagagaagatggtggccaagcagcacgggaaggtggtgatggccaaggtggatattgatgaccacacagacctcgccattgagtatgaggtgtcagcggtgcccactgtgctggccatgaagaatggggacgtggtggacaagtttgtgggcatcaaggatgaggatcagttggaggccttcctgaagaagctgattggctgac

SEQ ID NO: 14 - DNA, 502 bp, Artificial Sequence
  Description of Artificial Sequence/note = synthetic construct
atggctcagcgacttcttctgaggaggttcctggcctctgtcatctccaggaagccctctcagggtcagtggccacccctcacttccagagccctgcagaccccacaatgcagtcctggtggcctgactgtaacacccaacccagcccggacaatatacaccacgaggatctccttgacaacctttaatatccaggatggacctgactttcaagaccgagtggtcaacagtgagacaccagtggttgtggatttccacgcacagtggtgtggacccagcaagatcctggggccgaggttagagaagatggtggccaagcagcacgggaaggtggtgatggccaaggtggatattgatgaccacacagacctcgccattgagtatgaggtgtcagcggtgcccactgtgctggccatgaagaatggggacgtggtggacaagtttgtgggcatcaaggatgaggatcagttggaggccttcctgaagaagctgattggctgac

SEQ ID NO: 15 - protein, 4 aa, Artificial Sequence
  Description of Artificial Sequence/note = synthetic construct
CGPC

SEQ ID NO: 16 - protein, 4 aa, Artificial Sequence
  Description of Artificial Sequence/note = synthetic construct
XGPC

SEQ ID NO: 17 - protein, 4 aa, Artificial Sequence
  Description of Artificial Sequence/note = synthetic construct
CGPX

SEQ ID NO: 18 - protein, 105 aa, Artificial Sequence
  Description of Artificial Sequence/note = synthetic construct
MVKQIESKTAFQEALDAAGDKLVVVDFSATWSGPCKMIKPFFHSLSEKYSNVIFLEVDVDDCQDVASESEVKCMPTFQFFKKGQKVGEFSGANKEKLEATINELV

SEQ ID NO: 19 - protein, 105 aa, Artificial Sequence
  Description of Artificial Sequence/note = synthetic construct
MVKQIESKTAFQEALDAAGDKLVVVDFSATWCGPSKMIKPFFHSLSEKYSNVIFLEVDVDDCQDVASESEVKCMPTFQFFKKGQKVGEFSGANKEKLEATINELV

SEQ ID NO: 20 - DNA, 40 bp, Artificial Sequence
  Description of Artificial Sequence/note = synthetic construct
gaagcaggcccaggcagagcggaaagctgggaagaggcag

SEQ ID NO: 21 - protein, 5 aa, Artificial Sequence
  Description of Artificial Sequence/note = synthetic construct
TERKS

SEQ ID NO: 22 - RNA, 19 nt, Artificial Sequence
  Description of Artificial Sequence/note = synthetic construct
gccuuucuuucauucccuc

SEQ ID NO: 23 - RNA, 19 nt, Artificial Sequence
  Description of Artificial Sequence/note = synthetic construct
ugcaguccugguggccuga

SEQ ID NO: 24 - RNA, 19 nt, Artificial Sequence
  Description of Artificial Sequence/note = synthetic construct
cgaagcgagccaagggcaa

SEQ ID NO: 25 - DNA, 73 bp, Artificial Sequence
  Description of Artificial Sequence/note = synthetic construct
ctctcgatctgcttcaccatcttggctggaagcttgcggctaagatggtgaagcagattgagagtaatttttt

SEQ ID NO: 26 - DNA, 73 bp, Artificial Sequence
  Description of Artificial Sequence/note = synthetic construct
actaattcattaatggtggcttcaagctgaagcttgagcttgaggctactattaatgaattggtctatttttt

SEQ ID NO: 27 - DNA, 73 bp, Artificial Sequence
  Description of Artificial Sequence/note = synthetic construct
cccacaccacgtggctgagaagtcaactgaagcttgagttggcttctcagtcgcgtggtgtgggtcttttttt

SEQ ID NO: 28 - DNA, 73 bp, Artificial Sequence
  Description of Artificial Sequence/note = synthetic construct
tcttgctctcgatctgcttcaccatcttgaagcttgaggatggtgaagcggatcgggagcaggactgtttttt

SEQ ID NO: 29 - DNA, 79 bp, Artificial Sequence
  Description of Artificial Sequence/note = synthetic construct
gatcaaaaaattactctcaatctgcttcaccatcttagccgcaagcttccagccaagatggtgaagcagatcgagagcg

SEQ ID NO: 30 - DNA, 79 bp, Artificial Sequence
  Description of Artificial Sequence/note = synthetic construct
gatcaaaaaatagaccaattcattaatagtagcctcaagctcaagcttcagcttgaagccaccattaatgaattagtcg

SEQ ID NO: 31 - DNA, 79 bp, Artificial Sequence
  Description of Artificial Sequence/note = synthetic construct
gatcaaaaaaagacccacaccacgcgactgagaagccaactcaagcttcagttgacttctcagccacgtggtgtgggcg

SEQ ID NO: 32 - DNA, 79 bp, Artificial Sequence
  Description of Artificial Sequence/note = synthetic construct
gatcaaaaaacagtcctgctcccgatccgcttcaccatcctcaagcttcaagatggtgaagcagatcgagagcaagacg

SEQ ID NO: 33 - DNA, 73 bp, Artificial Sequence
  Description of Artificial Sequence/note = synthetic construct
caatgcgagcggagggatgcacagcctagaagcttgtgggttgtgcatctctccgttcgcattgcagtttttt

SEQ ID NO: 34 - DNA, 73 bp, Artificial Sequence
  Description of Artificial Sequence/note = synthetic construct
cttcttcaggaaggcctccaactgatccgaagcttgggattagttggaggccttcttggaggagctgtttttt

SEQ ID NO: 35 - DNA, 73 bp, Artificial Sequence
  Description of Artificial Sequence/note = synthetic construct
tgtatattgtccgggctgggttgggtgtgaagcttgatacccagcccagtccggataatatacaccatttttt

SEQ ID NO: 36 - DNA, 79 bp, Artificial Sequence
  Description of Artificial Sequence/note = synthetic construct
gatcaaaaaactgcaatgcgaacggagagatgcacaacccacaagcttctaggctgtgcatccctccgctcgcattgcg

SEQ ID NO: 37 - DNA, 79 bp, Artificial Sequence
  Description of Artificial Sequence/note = synthetic construct
gatcaaaaaacagctcctccaagaaggcctccaactaatcccaagcttcggatcagttggaggccttcctgaagaagcg

SEQ ID NO: 38 - DNA, 79 bp, Artificial Sequence
  Description of Artificial Sequence/note = synthetic construct
gatcaaaaaatggtgtatattatccggactgggctgggtatcaagcttcacacccaacccagcccggacaatatacacg

SEQ ID NO: 39 - DNA, 73 bp, Artificial Sequence
  Description of Artificial Sequence/note = synthetic construct
cctccgtggccgcacgctcgccctctgcgaagcttggtagggggcgagcgtgcggtcacgggggcgctttttt

SEQ ID NO: 40 - DNA, 73 bp, Artificial Sequence
  Description of Artificial Sequence/note = synthetic construct
atagccttccacagtgtgcacagcatccgaagcttggggtgctgtgtacgctgtggaaggctattattttttt

SEQ ID NO: 41 - DNA, 73 bp, Artificial Sequence
  Description of Artificial Sequence/note = synthetic construct
tcaaccccttcatgaagctgctgtcacagaagcttgtgtggcagtagcttcatgagggggttgatagtttttt

SEQ ID NO: 42 - DNA, 74 bp, Artificial Sequence
  Description of Artificial Sequence/note = synthetic construct
dgtggcagctggtgctcctcgccctcgccgaagcttgggcgggggcgaggggcaccggctgctaccgctttttt

SEQ ID NO: 43 - DNA, 73 bp, Artificial Sequence
  Description of Artificial Sequence/note = synthetic construct
gcaaggatatactcctgagatattctgcgaagcttggtaggatgtctcaggagtatattcttgccggtttttt

SEQ ID NO: 44 - DNA, 73 bp, Artificial Sequence
  Description of Artificial Sequence/note = synthetic construct
ctcggggctgctccgcgcccgccgggctgaagcttgagcctggtgggcgtggagcggccccgagctgtttttt

SEQ ID NO: 45 - DNA, 73 bp, Artificial Sequence
  Description of Artificial Sequence/note = synthetic construct
aaccaggatggtaatggctgtctgtaccgaagcttgggtataggcagccgttaccgtcctggttccatttttt

SEQ ID NO: 46 - DNA, 73 bp, Artificial Sequence
  Description of Artificial Sequence/note = synthetic construct
tcggctgcccccgccaacagagctgcccgaagcttggggcggctctgttggcgggggcggctgatggtttttt

SEQ ID NO: 47 - DNA, 73 bp, Artificial Sequence
  Description of Artificial Sequence/note = synthetic construct
aatgactctcatgtggtcattggctagggaagcttgcttagccgatgactacatgggagtcattcaatttttt

SEQ ID NO: 48 - DNA, 73 bp, Artificial Sequence
  Description of Artificial Sequence/note = synthetic construct
cgccgccgcctcctctccggcgccctctgaagcttgagggggcgtcggaggggaggcggcggcgtggtttttt

SEQ ID NO: 49 - DNA, 79 bp, Artificial Sequence
  Description of Artificial Sequence/note = synthetic construct
gatcaaaaaagcgcccccgtgaccgcacgctcgccccctaccaagcttcgcagagggcgagcgtgcggccacggaggcg

SEQ ID NO: 50 - DNA, 79 bp, Artificial Sequence
  Description of Artificial Sequence/note = synthetic construct
gatcaaaaaaataatagccttccacagcgtacacagcaccccaagcttcggatgctgtgcacactgtggaaggctatcg

SEQ ID NO: 51 - DNA, 79 bp, Artificial Sequence
  Description of Artificial Sequence/note = synthetic construct
gatcaaaaaactatcaaccccctcatgaagctactgccacacaagcttctgtgacagcagcttcatgaaggggttgacg

SEQ ID NO: 52 - DNA, 79 bp, Artificial Sequence
  Description of Artificial Sequence/note = synthetic construct
gatcaaaaaagcggtagcagccggtgcccctcgcccccgcccaagcttcggcgagggcgaggagcaccagctgccaccg

SEQ ID NO: 53 - DNA, 79 bp, Artificial Sequence
  Description of Artificial Sequence/note = synthetic construct
gatcaaaaaaccggcaagaatatactcctgagacatcctaccaagcttcgcagaatatctcaggagtatatccttgccg

SEQ ID NO: 54 - DNA, 79 bp, Artificial Sequence
  Description of Artificial Sequence/note = synthetic construct
gatcaaaaaacagctcggggccgctccacgcccaccaggctcaagcttcagcccggcgggcgcggagcagccccgagcg

SEQ ID NO: 55 - DNA, 79 bp, Artificial Sequence
  Description of Artificial Sequence/note = synthetic construct
gatcaaaaaatggaaccaggacggtaacggctgcctatacccaagcttcggtacagacagccattaccatcctggttcg

SEQ ID NO: 56 - DNA, 79 bp, Artificial Sequence
  Description of Artificial Sequence/note = synthetic construct
gatcaaaaaaccatcagccgcccccgccaacagagccgccccaagcttcgggcagctctgttggcgggggcagccgacg

SEQ ID NO: 57 - DNA, 79 bp, Artificial Sequence
  Description of Artificial Sequence/note = synthetic construct
gatcaaaaaattgaatgactcccatgtagtcatcggctaagcaagcttccctagccaatgaccacatgagagtcattcg

SEQ ID NO: 58 - DNA, 79 bp, Artificial Sequence
  Description of Artificial Sequence/note = synthetic construct
gatcaaaaaaccacgccgccgcctcccctccgacgccccctcaagcttcagagggcgccggagaggaggcggcggcgcg